(19) United States
(12) Patent Application Publication
(10) Pub. No.: US 2026/0027161 A1
(43) Pub. Date: Jan. 29, 2026

(54) BACTERIAL STRAINS FOR TREATING DISEASE

(71) Applicant: MICROBA IP PTY LTD, Brisbane (AU)

(72) Inventors: Páraic Ó. CUÍV, Brisbane (AU); Lutz KRAUSE, AU (AU)

(21) Appl. No.: 18/709,255

(22) PCT Filed: Nov. 11, 2022
(86) PCT No.: PCT/AU2022/051354

§ 371 Date: May 10, 2024

(30) Foreign Application Priority Data
Nov. 11, 2021 (AU) 2021903624

(51) Int. Cl. A61K 35/74; A61P 1/06; C12N 1/20; C12R 1/01

(52) U.S. Cl. CPC A61K 35/74; A61P 1/06; C12N 1/205; C12R 2001/01

(57) ABSTRACT

Disclosed are therapeutic compositions comprising bacterial strains and methods for the treatment or prevention of disease. More particularly, the present invention also discloses compositions comprising bacterial strains isolated from the human digestive tract and their use in the treatment or prevention of inflammatory and autoimmune disorders.

### FIELD OF THE INVENTION

This invention relates generally to the field of therapeutic compositions comprising bacterial strains and methods for the treatment or prevention of disease. More particularly, the present invention relates to compositions comprising bacterial strains isolated from the human digestive tract and their use in the treatment or prevention of inflammatory and autoimmune disorders.

### BACKGROUND OF THE INVENTION

The human gut microbiota contains more than 500-1000 different phylotypes belonging to a few bacterial phylum, including Firmicutes, Bacteroidetes, Proteobacteria, Fusobacteria, and Verrucomicrobia. The two major phyla, the Bacteroidetes and the Firmicutes, generally represent over 90% of the gut microbiota (Arumugam et al., 2011). The successful symbiotic relationships arising from bacterial colonisation of the human gut have yielded a wide variety of metabolic, structural, protective and other beneficial functions. The gut bacteria are key regulators of digestion along the gastrointestinal (GI) tract; with commensal bacterial playing an important role in the extraction, synthesis, and absorption of many nutrients and metabolites, including bile acids, lipids, amino acids, vitamins, and short-chain fatty acids (SCFAs). More recently, the immunological importance of the gut microbiota and their products in regulating the development, homeostasis, and function of innate and adaptive immune cells have been recognised (Brestoff and Atris, 2013).

It is now increasingly recognised that the gut microbiome regulates host intestinal mucosal immunity and predisposition to inflammation (Geva-Zatorsky et al., 2017; Kabat et al., 2014), opening new avenues for novel therapeutic interventions.

Dramatic changes in microbiota composition have been documented in many inflammatory and autoimmune disorders, including inflammatory bowel disease (IBD). In recognition of the potential positive effect that certain bacterial strains may have on the animal gut, various strains have been proposed for use in the treatment of various diseases. Certain strains, including Lactobacillus and Bifidobacterium strains, have been proposed for use in treating various extraintestinal inflammatory and autoimmune disorders (see, Goldin & Gorbach, 2008; Azad et al., 2013). However, the precise effects of particular bacterial strains locally at the GI tract and systemically throughout the body are unresolved. As a result, the relationships between different diseases and different bacterial strains in the human GI tract are yet to be clearly elucidated.

IBD (including the two major disease subtypes Crohn's disease (CD) and ulcerative colitis (UC)) is characterised by episodic and disabling inflammation of the GI tract. In 2017, it is estimated that 6.8 million people globally suffered from IBD, with the highest prevalence in the United States and Europe (GBD 2017; Inflammatory Bowel Disease Collaborators, 2019). Up to 20% of patients are diagnosed before the age of 16 and paediatric-onset IBD (PIBD) is associated with a more complicated and aggressive disease with adverse impacts on growth and psychosocial development.

There is currently no cure for IBD, and long-term clinical management requires effective therapeutics with an excellent safety profile. However, existing treatments show a range of deficiencies and remission is generally short. Moreover, IBD therapeutics are ineffective where early onset coupled with more aggressive disease result in progressive bowel damage and need for surgery. There is an urgent need to develop more effective and safe therapies to improve patient quality of life, maintain remission over long periods, reduce surgery and curtail individual and public health costs.

Existing treatments for IBD are sub-optimal with strong adverse effects, low compliance (50% average non-adherence rates (see, Chan et al., 2017)) and high cost. Furthermore, there is no effective solution to maintaining extended periods of disease-free remission. Mesalmine, one of the most widely used first line therapies for mild to moderate flares of ulcerative colitis and for maintenance of remission, has response rates between 40%-70% and remission rates of 15%-20% (Karagozian & Burakoff, 2007).

There is a requirement in the art for new methods of treating inflammatory and autoimmune disorders. There is also a requirement for the potential effects of gut bacteria to be characterised so that new therapies using gut bacteria can be developed.

### SUMMARY OF THE INVENTION

The present invention is predicated in part on the inventors identifying that bacterial strains of Intestinicoccus colisanans enhance or improve gut barrier function. Based on this consideration, it is proposed that strains of I. colisanans are particularly suited to therapeutic applications for treating and preventing inflammatory and autoimmune disorders, as described hereinafter.

The inventors have developed new compositions comprising a viable bacterial strain of the species Intestinicoccus colisanans that can be used for treating and preventing inflammatory and autoimmune disorders.

Accordingly, in one aspect the invention provides a cell of the Intestinicoccus colisanans strain deposited under accession number V21/015887 or V21/015888, or a derivative thereof.

In some embodiments, the cell is at least partially isolated.

In another aspect, the invention provides a biologically pure culture of the Intestinicoccus colisanans strain deposited under accession number V21/015887 or V21/015888, or a derivative thereof.

In another aspect, the present invention provides a composition comprising the cell or culture as described above and elsewhere herein.

In yet another aspect, the present invention provides a composition comprising a bacterial strain with a 16S rRNA sequence that is at least about 97.5%, 98%, 98.5% 99%, 99.1%, 99.2%, 99.3%, 99.4%, 99.5%, 99.6%, 99.7%, 99.8%, or 99.9%, identical to SEQ ID NO: 1; or with a 16S rRNA sequence represented by SEQ ID NO: 1. In some embodiments, the bacterial strain comprises two or more copies of a 16S rRNA sequence in its genome (e.g., two copies, three copies, four copies, five copies, six copies, seven copies, eight copies).

In some embodiments, the composition further comprises a pharmaceutically acceptable excipient, diluent, or carrier.

In yet another aspect, the present invention provides a pharmaceutical composition comprising a bacterial strain with a 16S rRNA sequence that is at least about 97%, 98%, 99%, 99.5%, 99.6%, 99.7%, 99.8%, or 99.9% identical to the 16S rRNA sequence of a bacterial strain of the species Intestinicoccus colisanans, together with a pharmaceutically acceptable carrier, diluent, or excipient.

Typically, the bacterial strain is at least partially isolated.

In some embodiments, the bacterial strain is live. In some alternative embodiments, the bacterial strain is dead.

In some embodiments, the compositions further comprise a prebiotic.

In some embodiments, the composition is formulated in a dried form. Typically, the composition is dried using techniques selected from lyophilisation, spray drying, fluidized bed drying, vacuum drying, or a combination thereof.

In some embodiments, the composition is formulated for oral administration.

In some embodiments, the bacterial strain produces an agent that attenuates or impairs signal transducer and activator of transcription 3 (STAT3) signalling in a cell.

In some embodiments of this type, the agent is a small molecule, peptide, or nucleotide. Typically, the agent is released by the bacterial strain.

In some embodiments, the agent binds specifically to any one of STAT3, JAK2, TYK2, or IL-23.

In some embodiments, the I. colisanans metabolizes one or more agents selected from the group comprising or consisting of starch, glucose, fructose, gluconate, lactose, trehalose, and lactaldehyde as a carbon source.

In another aspect, the present invention provides a method of restoring or improving gut barrier function in a subject, the method comprising administering to the subject a bacterial strain of the species I. colisanans, to thereby restore or improve gut barrier function.

In some preferred embodiments, restoring or improving gut barrier function is characterised by at least one of: (i) an increase in the quality and/or quantity of mucin; (ii) improvement in integrity of tight junction proteins; (iii) reduction in translocation of luminal contents into systemic circulation; or (iv) reduction of intestinal ulcers and/or intestinal wounds.

In some embodiments, the luminal contents includes lipopolysaccharide (LPS).

In some embodiments, the restoration or improvement in gut barrier function results in a reduction in systemic inflammation in the subject. In some embodiments of this type, the systemic inflammation is characterized by elevated levels of an inflammatory cytokine (e.g., IL-1P IL-8, IL-6, and TNF) in the subject as compared to the level of the inflammatory cytokine in a healthy subject.

In some embodiments, the I. colisanans bacterial strain stimulates PBMCs to produce the cytokines IL-10 and IL-12 at a ratio equal or greater than 5. For example, the I. colisanans bacterial strain may stimulate PBMCs to produce the cytokines IL-10 and IL-12 at a ratio equal or greater than 10, 15, 20, 25, or 30.

In yet another aspect, the present invention provides a method of maintaining gut barrier function in a subject, the method comprising administering to the subject a bacterial strain of the species I. colisanans, to thereby maintain gut barrier function in the subject.

In yet another aspect, the present invention provides a method of reducing inflammation in a subject, the method comprising administering to the subject a bacterial strain of the strain I. colisanans, to thereby reduce inflammation in the subject.

In some embodiments, the inflammation is local to the gut environment, or systemic inflammation.

In another aspect, the present invention provides a method of inducing or enhancing mucosal healing in a subject, the method comprising administering to the subject a bacterial strain of the species I. colisanans in an amount sufficient to induce epithelial cell migration, proliferation and/or differentiation, to thereby induce mucosal healing in the subject.

In some embodiments, mucosal healing in the subject can be measured using one or more fecal or serum markers. By way of an illustrative example, one or more fecal markers may be selected from the group comprising calprotectin, lactoferrin, metalloproteinase (MMP)-9, and lipocalin-2.

In some embodiments, the bacterial strain reduces inflammation by attenuating the NFκB pathway. In some embodiments of this type, the bacterial strain inhibits the production of one or more transcription factors, cytokines, or chemokines selected from the group comprising NFκB, TNF, IFN-γ, IL-1β, IL-8, and MCP-1.

In yet another aspect, the present invention provides methods of blocking or otherwise inhibiting STAT3 signalling in a target cell, the method comprising contacting the cell with at least a soluble component of a bacterial cell preparation of the species I. colisanans, to block or otherwise inhibit STAT3 signalling in the cell. Typically, the method of this aspect is performed in vitro.

In some embodiments, the target cell is selected from a reporter cell (e.g., a HEK cell), an immune cell (e.g., a Th17 immune cell), an epithelial cell, or an endothelial cell. In some embodiments, the target cell is a mammalian cell, and preferably, a human cell.

In some embodiments, the bacterial cell preparation is a bacterial cell culture. The soluble component may therefore comprise, consist, or consist essentially of, the soluble fraction of the bacterial cell culture (e.g., the cell culture supernatant). The soluble component may further comprise some insoluble components of the bacterial cell culture. For example, the soluble component may include substantially all of the bacterial culture. Preferably, the soluble component is substantially depleted of bacterial cells.

In some alternative embodiments, the bacterial cell preparation is a bacterial cell lysate. In exemplary embodiments of this type, the soluble component may relate to the soluble fraction of the cell lysate. A soluble fraction can suitably be achieved by any method, including by centrifugation.

In still yet another aspect, the present invention provides a method of blocking or otherwise inhibiting STAT3 signalling in a cell, the method comprising administering a bacterial strain of the species I. colisanans to the subject, thereby blocking or otherwise inhibiting STAT3 signalling in the cell. Typically, the methods of this aspect are performed in vivo.

In some embodiments, the cell is an immune cell (e.g., a Th17 immune cell) or epithelial cell.

In some embodiments, the cell is an epithelial cell, and the bacterial strain or a metabolite produced by the bacterial strain increases the production of IL-22 in the subject.

In some embodiments, the bacterial strain produces a molecule that is a direct inhibitor or an indirect inhibitor of STAT3. For example, the bacterial strain may produce a metabolite that directly inhibits at least one of an IL-23 polypeptide, a JAK2 polypeptide, a TYK2 polypeptide, or a STAT3 polypeptide.

In some embodiments, the bacterial strains used in the methods described above and elsewhere herein produces the metabolite acetate.

In some embodiments the bacterial strain has a 16S rRNA sequence that is at least about 97.5%, 98.5%, 99%, 99.1%, 99.2%, 99.3%, 99.4%, 99.5%, 99.6%, 99.7%, 99.8%, 99.9% identical to the 16S rRNA sequence of a bacterial strain of I. colisanans.

In some alternative embodiments, the bacterial strain has a 16S rRNA sequence that is at least about 97.5%, 98.5%, 99%, 99.1%, 99.2%, 99.3%, 99.4%, 99.5%, 99.6%, 99.7%, 99.8%, 99.9% identical to SEQ ID NO: 1 or when the bacterial strain has the 16S rRNA gene sequence represented by SEQ ID NO: 1. In some embodiments, the bacterial strain comprises two or more copies (e.g., two copies, three copies, four copies, five copies, six copies, seven copies, eight copies) independently selected from the 16S rRNA sequences set forth in SEQ ID NO: 1. In some alternative embodiments, the bacterial strain has a 16S rRNA sequence that is at least about 97.5%, 98.5%, 99%, 99.1%, 99.2%, 99.3%, 99.4%, 99.5%, 99.6%, 99.7%, 99.8%, 99.9% identical to SEQ ID NO: 2 or when the bacterial strain has the 16S rRNA gene sequence represented by SEQ ID NO: 2. In some embodiments, the bacterial strain comprises two or more copies (e.g., two copies, three copies, four copies, five copies, six copies, seven copies, eight copies) independently selected from the 16S rRNA sequences set forth in SEQ ID NO: 2.

In some embodiments, the bacterial strain is the I. colisanans strain deposited under accession number V21/015887 or V21/015888, or a derivative thereof.

Preferably, the bacterial strain is at least partially isolated.

In some embodiments, the bacterial strain is formulated as a pharmaceutical composition, further comprising a pharmaceutically acceptable carrier, diluent or excipient. In some embodiments, the pharmaceutical composition is a dry composition. In some embodiments, the dry composition is selected from the group consisting of particles, granules, and powder. By way of an illustrative example the pharmaceutical composition may be lyophilised, spray dried, fluidized bed dried, vacuum dried, or a combination thereof.

In some embodiments, the pharmaceutical composition is formulated for oral administration.

In still yet other aspects, the present invention provides a method of treating an inflammatory or autoimmune disorder in a subject, the method comprising administering an effective amount of a bacterial strain of I. colisanans to the subject, to thereby treat or prevent the inflammatory or autoimmune disorder.

In some embodiments, the inflammatory or autoimmune disorder is selected from the group comprising an inflammatory bowel disease (such as Crohn's disease or ulcerative colitis); asthma (such as allergic asthma or neutrophilic asthma); fatty liver disease (such as non-alcoholic fatty liver disease (NAFLD)); ankylosing spondylitis; systemic lupus erythematosus (SLE); scleroderma; Sjogren's syndrome; and vasculitis. Preferably, the inflammatory or autoimmune disorder is an inflammatory bowel disease (IBD).

In some embodiments, the bacterial strain blocks or otherwise inhibits STAT3 signalling in at least a cell of the subject. Typically, the cell is an epithelial cell, endothelial cell, or an immune cell (e.g., a Th17 immune cell).

In some embodiments, the bacterial strain has a 16S rRNA sequence that is at least about 98%, 98.5, 99%, 99.1%, 99.2%, 99.3%, 99.4%, 99.5%, 99.6%, 99.7%, 99.8%, or 99.9% identical to the 16S rRNA sequence of a bacterial strain of I. colisanans.

Alternatively, in some embodiments the bacterial strain has a 16S rRNA sequence that is at least about 98%, 98.5, 99%, 99.1%, 99.2%, 99.3%, 99.4%, 99.5%, 99.6%, 99.7%, 99.8%, or 99.9% identical to SEQ ID NO: 1, or when the bacterial strain has the 16S rRNA gene sequence represented by SEQ ID NO: 1. Alternatively, the bacterial strain may have a 16S rRNA sequence that is at least about 98%, 98.5, 99%, 99.1%, 99.2%, 99.3%, 99.4%, 99.5%, 99.6%, 99.7%, 99.8%, or 99.9% identical to SEQ ID NO: 2, or when the bacterial strain has the 16S rRNA gene sequence represented by SEQ ID NO: 2.

In some alternative embodiments, the bacterial strain may have a 16S rRNA sequence that is at least about 98%, 98.5, 99%, 99.1%, 99.2%, 99.3%, 99.4%, 99.5%, 99.6%, 99.7%, 99.8%, or 99.9% identical to any one of SEQ ID NOs: 7-10, or when the bacterial strain has the 16S rRNA gene sequence represented by any one of SEQ ID NOs: 7-10.

Preferably, the bacterial strain is at least partially isolated.

In some embodiments, the bacterial strain is formulated as a pharmaceutical composition, together with a pharmaceutically acceptable carrier, diluent, and/or excipient. In some embodiments, the composition is a dry composition selected from the group consisting of particles, granules, and powder. For example, the composition may be lyophilised. Alternatively, the composition may be spray dried, fluidized bed dried, or vacuum dried.

In some embodiments, the composition is formulated for oral administration.

In one aspect, the present invention provides a composition comprising a bacterial strain of the genus Intestinicoccus for use in therapy.

In another aspect, the present invention provides a composition comprising a bacterial strain of I. colisanans, for use in therapy. In some of the same embodiments and some other embodiments, the bacterial strain has a 16S rRNA sequence that is at least about 98%, 98.5, 99%, 99.1%, 99.2%, 99.3%, 99.4%, 99.5%, 99.6%, 99.7%, 99.8%, or 99.9% identical to the 16S rRNA sequence of a bacterial strain of I. colisanans.

Alternatively, the bacterial strain may have a 16S rRNA sequence that is at least about 98%, 98.5, 99%, 99.1%, 99.2%, 99.3%, 99.4%, 99.5%, 99.6%, 99.7%, 99.8%, or 99.9% identical to SEQ ID NO: 1, or when the bacterial strain has the 16S rRNA gene sequence represented by SEQ ID NO: 1.

Alternatively, the bacterial strain may have a 16S rRNA sequence that is at least about 98%, 98.5, 99%, 99.1%, 99.2%, 99.3%, 99.4%, 99.5%, 99.6%, 99.7%, 99.8%, or 99.9% identical to SEQ ID NO: 2, or when the bacterial strain has the 16S rRNA gene sequence represented by SEQ ID NO: 2.

Alternatively, the bacterial strain may have a 16S rRNA sequence that is at least about 98%, 98.5, 99%, 99.1%, 99.2%, 99.3%, 99.4%, 99.5%, 99.6%, 99.7%, 99.8%, or 99.9% identical to any one of SEQ ID NOs: 7-10, or when the bacterial strain has the 16S rRNA gene sequence represented by any one of SEQ ID NOs: 7-10.

In yet another aspect, the present invention provides a composition comprising a bacterial strain of the genus Intestinicoccus, for use in the treatment or prevention of an inflammatory or autoimmune disorder.

In still yet another aspect, the present invention provides a composition comprising a bacterial strain of I. colisanans, for use in the treatment or prevention of an inflammatory or autoimmune disorder.

In some embodiments, the bacterial strain is the I. colisanans strain, or a derivative thereof.

In some embodiments, the inflammatory or autoimmune disorder is selected from an inflammatory bowel disease (such as Crohn's disease or ulcerative colitis); asthma (such as allergic asthma or neutrophilic asthma); arthritis (such as rheumatoid arthritis, osteoarthritis, psoriatic arthritis, or juvenile idiopathic arthritis); fatty liver disease (such as nonalcoholic fatty liver disease (NAFLD)); ankylosing spondylitis; psoriasis; systemic lupus erythematosus (SLE); scleroderma; Sjogren's syndrome; and type 1 diabetes mellitus. In some preferred embodiments, the inflammatory or autoimmune disorder is an inflammatory bowel disease (IBD).

In one aspect, the present invention provides a composition for use in treating an inflammatory or autoimmune disorder, the composition comprising a bacterial strain of I. colisanans; and an ancillary treatment agent.

In some embodiments, the ancillary treatment agent is an anti-inflammatory agent. By way of an illustrative example, the anti-inflammatory agent is selected from the group comprising 5-aminosalicylates, corticosteroids, azathioprine, or a combination thereof. In some other embodiments, the ancillary treatment is an antibody (e.g., a monoclonal antibody). By way of an illustrative example, the antibody may be selected from infliximab, adalimumab, golimumab, certolizumab pegol, natalizumab, and vedolizumab.

In another aspect, the present invention provides a composition for use in treating an inflammatory or autoimmune disorder, the composition comprises a bacterial strain of I. colisanans; and a nutritional supplement. In embodiments of this type, the nutritional supplement improves engraftment of the bacterial stain.

In some related aspects, the technology described herein provides bacterial species and compositions comprising them in for form of probiotics. Preferably, such probiotics are effective to improve intestinal microbial ecology, alleviate symptoms of microbial dysbiosis, promote wellness, and/or treat or prevent inflammatory and/or autoimmune disorders.

### BRIEF DESCRIPTION OF THE FIGURES

The following figures form part of the present specification and are included to further demonstrate certain aspects of the present disclosure. The disclosure may be better understood by reference to one or more of these figures in combination with the detailed description of specific embodiments presented herein.

FIG. 1 provides graphical representations of the association between I. colisanans and representative inflammatory and/or autoimmune disorders. (A) Using high resolution gut metagenomic data of 6,020 subjects (MDD), we identified I. colisanans as being significantly less prevalent in a range of inflammatory and autoimmune disorders (striped bars) compared to healthy individuals (black bar) (P<0.05, Fisher's exact test). (B) The strongest reduction was observed for IBD, including both major subtypes Crohn's disease (CD) and ulcerative colitis (UC). These observations were validated in an independent IBD cohort previously published by Harvard (Franzosa et al., 2019).

FIG. 2 provides photographic and graphical representations of the morphology and phylogeny of I. colisanans. (A) Gram-staining I. colisanans isolate showing morphology. (B) A phylogenetic tree constructed with an alignment of 120 bacteria specific single copy marker genes from high quality reference genomes (GTDB r89). Nonparametric bootstrap values calculated from 1000 iterations. Intestinococcus was previously designated UBA1417.

FIG. 3 provides graphical representations that I. colisanans does not affect healthy gut function in naïve C57BI/6 mice. (A) Overview of the model used to assess the effect of I. colisanans on naïve C57Bl/6 mice. (B) Treatment with I. colisanans has little effect on body weight of naïve animals. (C)-(D) Treatment with I. colisanans has no effect on colon length or colon weight/length ratio relative to vehicle treated controls in naïve animals. (E)-(F) Treatment with I. colisanans has no effect on epithelial injury, inflammation, hypervascularization relative to vehicle treated controls in naïve animals. (G) Treatment with I. colisanans has no effect on gut histology relative to vehicle treated controls in naïve animals. All data reported as mean and standard deviation. ns, not significant.

FIG. 4 provides graphical representations that I. colisanans restores gut barrier function. (A) Overview of the DSS mouse model used to assess the therapeutic efficacy of I. colisanans. (B) Effect of daily treatment of vehicle, prednisone and I. colisanans in healthy and DSS treated mice. All treatment groups were compared to the DSS+vehicle group. Significance was determined using a two-way ANOVA with Tukey's test for multiple comparison. (C) Endoscopic assessment of colitis as assessed on days 1, 2 and 6. All groups were compared to the DSS+vehicle group for each individual day using a Kruskal-Wallis test with Dunn's correction for multiple comparisons (day 1) or one-way Anova with Dunnett's correction for multiple comparison (day 2, 6) as appropriate. All data presented as mean and standard deviation. (D) Representative gut histology images of C57Bl/6 mice treated with vehicle, prednisone, or I. colisanans. (E) DSS treatment results in an increase in the histopathological score that is ameliorated by treatment with prednisone, F. prausnitzii A2-165 and I. colisanans. All data presented as mean and standard deviation. All groups were compared to the DSS+vehicle group and significance was determined using a one-way ANOVA with Dunnett's test for multiple comparison. (F) DSS treatment results in an increase in epithelial injury that is ameliorated by treatment with prednisone and I. colisanans. All data presented as mean and standard deviation. All groups were compared to the DSS+vehicle group using a Kruskal-Wallis test with Dunn's correction for multiple comparisons. (G) Total histopathological scores of mice, (H) epithelial sub-score of total histopathological scores of all mice, (I) inflammation sub-score of total histopathological scores of all mice. All groups were compared against the DSS control group using ordinary One-way ANOVA with Dunnett's multiple comparisons test. D'Agostino-Pearson Omnibus tests were applied to all data to test for normal distribution, with all data passing this test. Brown-Forsyth tests were applied to all data to test for significant differences in group standard deviations, with all data passing this test. All groups are visually shown as points representing individual mice, with columns and error bars representing each group's mean and standard deviation. After correction for multiple comparisons, the following annotations for statistical significance are used; *, p<0.05; **, p<0.01; ***, p<0.001; ****, p<0.0001. (J) Disease activity index scores of healthy or DSS-treated mice administered I. colisanans MH27-2 at the indicated doses and formulations, or control drugs minocycline and prednisone. All groups treated with I. colisanans MH27-2 or control drugs were compared against DSS-vehicle controls using Brown-Forsythe and Welch ANOVA with Dunnett's T3 multiple comparisons tests. (K) Mouse body weights expressed as percentage of original body weights of healthy or DSS-treated mice administered I. colisanans MH27-2 at the indicated doses and formulations, or control drugs minocycline and prednisone. All groups treated with I. colisanans MH27-2 or control drugs were compared against DSS-vehicle controls using ordinary one-way ANOVA with Dunnett's multiple comparisons tests. (L) Fecal occult blood scores of healthy or DSS-treated mice administered I. colisanans MH27-2 at the indicated doses and formulations, or control drugs minocycline and prednisone. All groups treated with I. colisanans MH27-2 or control drugs were compared against DSS-vehicle controls using Brown-Forsythe and Welch ANOVA with Dunnett's multiple comparisons tests. (M-P) Histopathological total scores and sub-scores of healthy or DSS-treated mice administered I. colisanans MH27-2 at the indicated doses and formulations, or control drugs minocycline and prednisone. All groups treated with I. colisanans MH27-2 or control drugs were compared against DSS-vehicle controls using ordinary one-way ANOVA with Dunnett's multiple comparisons tests. All groups are visually shown as points representing individual mice, with columns and error bars representing each group's mean and standard deviation. For all statistical analyses, D'Agostino-Pearson omnibus tests were used to determine if data were normally distributed, with all data shown passing this test. Brown-Forsyth tests were used to determine if standard deviations were significantly different between groups, and if this test was significant, the Brown-Forsyth and Welch ANOVA tests were used instead of the ordinary one-way ANOVA. In this case, the Dunnett's T3 multiple comparisons test was used instead of the Dunnett's test. After correction for multiple comparisons, the following annotations for statistical significance are used: *, p<0.05; **, p<0.01; ***, p<0.001; ****, p<0.0001.

FIG. 5. (A) Overview of the SKG model used to assess the therapeutic efficacy of I. colisanans. (B) Curdlan treatment results in an increase in the histopathological score that is ameliorated by treatment with anti-IL-23 antibody and I. colisanans. All data presented as mean and standard deviation. All groups were compared to the curdlan+vehicle group using an ANOVA with Dunnett's test for multiple comparisons. (C) Curdlan treatment results in an increase in IL-6 and IL-12p70 that is ameliorated by treatment with I. colisanans. All data presented as mean and standard deviation. For IL-6, all groups were compared to the curdlan+vehicle group using an ordinary one-way ANOVA with Dunnett's test for multiple comparisons. For IL-12p70, all groups were compared to the DSS+vehicle group using a Kruskal-Wallis test with Dunn's correction for multiple comparisons. For IL-6, all groups were compared to the curdlan+vehicle group using an ordinary one-way ANOVA with Dunnett's test for multiple comparisons. For all data, ns: not significant; *, p<0.05; **, p<0.01; ***, p<0.001****, p<0.0001. (E) Overview of the TNBS model used to assess the therapeutic efficacy of I. colisanans MH27-2. (F) TNBS treatment results in an increase in the macroscopic damage histopathological score that is ameliorated by treatment with I. colisanans MH27-2 and cyclosporine A. The TNBS+vehicle group were compared to the I. colisanans MH27-2 and cyclosporine A treated groups using an Ordinary One-Way ANOVA test with Sidek's correction for multiple comparisons. (G) Treatment with I. colisanans MH27-2 results in significant improvements in the ulcers/inflammation scores. The TNBS+vehicle group were compared to the I. colisanans MH27-2 and cyclosporine A treated. (H) TNBS treatment results in an increase in the total histopathological score that is ameliorated by treatment with I. colisanans MH27-2 and cyclosporine A. The TNBS+vehicle group were compared to the I. colisanans MH27-2 and cyclosporine A treated groups using Brown-Forsythe and Welch ANOVA tests with Dunnett's T3 correction for multiple comparisons. (I-L) Treatment with I. colisanans MH27-2 results in significant improvements in the extent of inflammation, erosion or ulceration, epithelial regeneration and percent involvement scores. The TNBS+vehicle group were compared to the I. colisanans MH27-2 and cyclosporine A treated groups using the Kruskal-Wallis test with Dunn's multiple comparison test. (M) Treatment with I. colisanans MH27-2 results in significant reduction in IL-6 concentration. The TNBS+vehicle group was compared to the I. colisanans MH27-2 and cyclosporine A treated groups. The data was log transformed and analysed using a using the Kruskal-Wallis test with Dunn's multiple comparison test an Ordinary One-Way ANOVA test with Sidek's correction for multiple comparisons. (ns: not significant; *, p<0.05; **, p<0.01; ***, p<0.001; ****, p<0.0001).

FIG. 6 provides a graphical representation of I. colisanans MH27-1 and MH27-2 suppressing IL-6 mediated STAT3 activation in vitro. STAT3 signalling is inhibited when HEKBlue IL-6 reporter cells are treated with cell-free supernatant raw or <3KDa fractionated culture supernatant of (A) I. colisanans MH27-1 and (B) MH27-2 at 10% v/v (unpaired t-test, n=18), (C) MH27-3 (unpaired t-test, n=18), (D), MH27-4 v (unpaired t-test, n=18), (E) MH27-5 v (unpaired t-test, n=18) and (F) R. bromii MCB950 (unpaired t-test, n=12). STAT3 activation by IL-6 trans signalling is inhibited cell-free supernatant raw of I. colisanans MH27-2.

FIG. 7 provides a graphical representation showing that I. colisanans promote the migration of human gut epithelial cells. (A) Transwell migration assays were employed to study the effect of sterile culture supernatant extracts from I. colisanans on the migration of HCT116 colon cancer cells. In serum-starved conditions (0.5% FBS), the addition of I. colisanans 0.5× extract to the bottom of the chamber significantly increased the movement of HCT116 cells to the basolateral side compared to the medium extract control. (Untreated and medium control n=6 technical replicates; I. colisanans n=4 technical replicates, for 3 biological replicates each; Unpaired t-test, two-tailed P>0.0001). (B) As a second readout for cell migration, the Incucyte scratch wound assay was performed. The relative wound confluence was measured every two hours after the HCT116 cell monolayer was scratched. 24 hours post scratch, serum-starved HCT116 cells incubated in 0.3× extract from I. colisanans strains MH27-2 showed significantly higher wound confluence, compared to cells treated with medium extract. (One-way Anova with Dunnett's multiple comparisons test, *, p<0.05).

FIG. 8 provides graphical representation of MH27 suppressing IL-23-mediated activation of STAT3. STAT3 signalling is inhibited when HEK-Blue™ IL 23 reporter cell lines are treated with 25% cell-free supernatant or <3 kDa size fractionated I. colisanans but not R. bromii supernatant and similarly prepared YG/P medium. Samples were compared using an unpaired t-test (***, p<0.001; ns not significant). These data shown are the results of three independent experiments combined. Technical and biological replicates of raw and <3 kDa fractions were pooled together.

FIG. 9. After treatment with IFNγ (48 hour treatment (grey area)), culture supernatant from I. colisanans MH27-2 ameliorated the reduction in TEER relative to the YG/V medium control at 168, 192, 216, 240, 288, 312 and 336 hours (p<0.05 by two-way ANOVA test). (▴) untreated, (⋄) IFNγ+I. colisanans MH27-2 extract, (●) IFNγ+medium extract, (▪) IFNγ.

FIG. 10. Treatment with I. colisanans MH27-2 culture supernatant extract potentiates IL-6 (96-hour treatment (grey area)), mediated reduction in TEER relative to the medium control in a dose dependent manner. Statistical significance was determined by unpaired t test. (▴) untreated, (⋄) IL-6+I. colisanans MH27-2 extract, (●) IL-6+medium extract, (▪) IL-6.

FIG. 11 provides graphical representations showing re-treatment with I. colisanans MH27 extracts ameliorates IFNγ-induced reductions in ZO1 expression in T84 cells. (A) T84 cells were pre-treated with extracts of YG/V media control or I. colisanans MH27-1, MH27-2, or MH27-3 (1×) or left untreated for 18 h. The cells were then stimulated with recombinant IFNγ for 48 h, stained for ZO1 and imaged by confocal microscopy. Scale bars represent 10 μm. (B) Quantification of ZO1 relative brightness normalised to unstimulated cells revealing that pre-treatment with I. colisanans MH27 extracts significantly mitigates IFNγ induced reductions in ZO1 expression in T84 cells. Data are means±SD of one experiment and four replicates. One-way ANOVA with Dunnett's correction for multiple comparisons, *p<0.05, **p<0.01, ***p<0.001, ****p<0.0001.

FIG. 12 provides graphical representations to show that pre-treatment with I. colisanans MH27 fractions ameliorates IFNγ-induced reductions in ZO1 expression in T84 cells. Effect of fractionated (A) YG/V and (B) I. colisanans MH27-2 extracts on ZO1 expression. Scale bars represent 40 μm. (C) Quantification o^{f} ZO1 relative brightness normalised to unstimulated cells revealing that pre-treatment wi^{t}h 15- and 30% fractions of I. colisanans MH27-2 extracts significantly mitigates IFNγ induced reductions in ZO1 expression in T84 cells. Data are means±SD of one experiment and four replicates. One-way ANOVA with Dunnett's correction for multiple comparisons, ***p<0.001, ****p<0.0001.

FIG. 13 provides graphical representations to show I. colisanans MH27-produced metabolites ameliorate IFNγ-induced reductions in ZO1 expression in T84 cells. (A) T84 cells were pre-treated with ornithine, indole-3-acrylic acid (IAyA), or indole-3-proprionic acid (IPA) or left untreated for 18 h. The cells were then stimulated with recombinant IFNγ for 48 h, stained for ZO1 and imaged by confocal microscopy. Scale bars represent 10 μm. (B) Quantification of ZO1 relative brightness normalised to unstimulated cells revealing that pre-treatment with all investigated metabolites significantly mitigates IFNγ induced reductions in ZO1 expression in T84 cells. Data are means±SD of one experiment and four replicates. One-way ANOVA with Dunnett's correction for multiple comparisons, **p<0.01, ***p<0.001, ****p<0.0001. (C) Cyclo(-Phe-Pro) (CPP) suppresses IL-23 mediated activation of STAT3 relative to the vehicle control. Data are means±SD of three experiments. Data was analysed using an unpaired t-test. **p<0.0001.

FIG. 14. IL-10/IL-12 ratio of PBMCs stimulated with I. colisanans. Data are means and SD of a single experiment.

FIG. 15. NF-κB activation is suppressed when the LS174T-NF-κB cell line is treated with 25% cell-free or <3 kDa size fractionated I. colisanans supernatant as compared to similarly prepared medium (A-C) R. bromii MCB950 cell free supernatant does not suppresses NF-κB activation (D) Samples were compared using an unpaired t-test (*, p<0.05; **, p<0.01; ***, p<0.001; ****, p<0.0001 ns not significant). The data shown are the results of 3 independent experiments combined.

FIG. 16. Gating strategy of the CytoFLEX SRT Benchtop Cell Sorter used to isolate I. colisanans MH27-4 (A), MH27-5 (B), MH27-6 (C) and R. bromii MCB950 (D).

### BRIEF DESCRIPTION OF THE SEQUENCES

| SEQ | |
|---|---|
| ID NO: | Sequence Description |
| 1 | 16S rRNA sequence of I. colisanans strain MH27-1 |
| 2 | 16S rRNA sequence of I. colisanans strain MH27-2 |
| 3 | Genome sequence of I. colisanans strain MH27-1 (scaffold 1) |
| 4 | Genome sequence of I. colisanans strain MH27-1 (scaffold 2) |
| 5 | Genome sequence of I. colisanans strain MH27-2 (scaffold 1) |
| 6 | Genome sequence of I. colisanans strain MH27-2 (scaffold 2) |
| 7 | 16S rRNA sequence of I. colisanans strain MH27-3 |
| 8 | 16S rRNA sequence of I. colisanans strain MH27-4 |
| 9 | 16S rRNA sequence of I. colisanans strain MH27-5 |
| 10 | Partial 16S rRNA sequence of I. colisanans strain MH27-6 |
| 11 | Genome sequence of I. colisanans strain MH27-3 (scaffold 1) |
| 12 | Genome sequence of I. colisanans strain MH27-3 (scaffold 2) |
| 13 | Genome sequence of I. colisanans strain MH27-3 (scaffold 3) |
| 14 | Genome sequence of I. colisanans strain MH27-3 (scaffold 4) |
| 15 | Genome sequence of I. colisanans strain MH27-4 (scaffold 1) |
| 16 | Genome sequence of I. colisanans strain MH27-4 (scaffold 2) |
| 17 | Genome sequence of I. colisanans strain MH27-4 (scaffold 3) |
| 18 | Genome sequence of I. colisanans strain MH27-4 (scaffold 4) |
| 19 | Genome sequence of I. colisanans strain MH27-5 (scaffold 1) |
| 20 | Genome sequence of I. colisanans strain MH27-5 (scaffold 2) |
| 21 | Genome sequence of I. colisanans strain MH27-5 (scaffold 3) |
| 22 | Genome sequence of I. colisanans strain MH27-5 (scaffold 4) |

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

The term “about” as used herein refers to the usual error range for the respective value readily known to the skilled person in this technical field. Reference to “about” a value or parameter herein includes (and describes) embodiments that are directed to that value or parameter perse.

As used herein, the term “administering,” refers to the placement of an agent (e.g., bacteria) as disclosed herein into a subject by a method or route which results in at least partial delivery of the agent at the desired site. Compositions comprising the compounds disclosed herein can be administered by any appropriate route which results in an effective biological activity or therapeutic effect in the subject. In some embodiments, administration comprises physical human activity (e.g., an injection, act of ingestion, an act of application, and/or manipulation of a delivery device or machine). Such activity can be performed (e.g., by a medical professional and/or the subject being treated).

Specifically, as used herein “administer” and “administration” encompasses embodiments in which one person directs another to consume live bacteria, dead bacteria, spent mediums derived from bacteria, cell pellets of bacteria, purified metabolites produced by bacteria, purified proteins produced by bacteria, prebiotics, small molecules, or combinations thereof in a certain manner and/or for a certain purpose independently of or in variance to any instructions received from a second person. Non-limiting examples of embodiments include the situation in which one person directs another to consume live bacteria, dead bacteria, spent mediums derived from bacteria, cell pellets of bacteria, purified metabolites produced by bacteria, purified proteins produced by bacteria, prebiotics, small molecules, or combinations thereof in a certain manner and/or for a certain purpose include when a physician prescribes a course of conduct and/or treatment to a patient, when a parent commands a minor user (such as a child) to consume such a product, when a trainer advises a user (such as an athlete) to follow a particular course of conduct and/or treatment, or when a manufacturer, distributer, or marketer recommends conditions of use to an end user, for example through advertisements or labeling on packing or on other materials provided in association with the sale or marketing of a product. In some embodiments, the disclosed compositions can be administered orally, intravenously, intramuscularly, intrathecally, subcutaneously, sublingually, buccally, rectally, vaginally, by the ocular route, by the optic route, nasally, via inhalation, by nebulization, cutaneously, transdermally, or combinations thereof, and formulated for delivery with a pharmaceutically acceptable excipient, carrier or diluent. Of note, although the disclosed compositions encompass multiple formulations and modes of delivery for treatments to ameliorate dysbiosis and its sequelae, it should be noted that live biotherapeutic products such as probiotics are not typically administered intravenously, intramuscularly, or intraperitoneally. These modes of delivery would likely be reserved for small-molecule products of bacterial metabolism.

The terms “administration concurrently” or “administering concurrently” or “co-administering” and the like refer to the administration of a single composition containing two or more actives, or the administration of each active as separate compositions and/or delivered by separate routes either contemporaneously or simultaneously or sequentially within a short enough period of time that the effective result is equivalent to that obtained when all such actives are administered as a single composition. By “simultaneously” is meant that the active agents are administered at substantially the same time, and desirably together in the same formulation. By “contemporaneously” it is meant that the active agents are administered closely in time, e.g., one agent is administered within from about one minute to within about one day before or after another. Any contemporaneous time is useful. However, it will often be the case that when not administered simultaneously, the agents will be administered within about one minute to within about eight hours and suitably within less than about one to about four hours. When administered contemporaneously, the agents are suitably administered at the same site on the subject. The term “same site” includes the exact location, but can be within about 0.5 to about 15 centimeters, preferably from within about 0.5 to about 5 centimeters. The term “separately” as used herein means that the agents are administered at an interval, for example at an interval of about a day to several weeks or months. The active agents may be administered in either order. The term “sequentially” as used herein means that the agents are administered in sequence, for example at an interval or intervals of minutes, hours, days or weeks. If appropriate the active agents may be administered in a regular repeating cycle.

The term “agent” includes a compound that induces a desired pharmacological and/or physiological effect. The term also encompasses pharmaceutically acceptable and pharmacologically active ingredients of those compounds specifically mentioned herein including but not limited to salts, esters, amides, prodrugs, active metabolites, analogs and the like. When the above term is used, then it is to be understood that this includes the active agent per se as well as pharmaceutically acceptable, pharmacologically active salts, esters, amides, prodrugs, metabolites, analogs, etc. The term “agent” is not to be construed narrowly but extends to small molecules, proteinaceous molecules such as peptides, polypeptides and proteins as well as compositions comprising them and genetic molecules such as RNA, DNA and mimetics and chemical analogs thereof as well as cellular agents. The term “agent” includes a cell that is capable of producing and secreting a polypeptide referred to herein as well as a polynucleotide comprising a nucleotide sequence that encodes that polypeptide. Thus, the term “agent” extends to nucleic acid constructs including vectors such as viral or non-viral vectors, expression vectors and plasmids for expression in and secretion in a range of cells.

The “amount” or “level” of a biomarker is a detectable level in a sample. These can be measured by methods known to one skilled in the art and also disclosed herein. The expression level or amount of biomarker assessed can be used to determine the response to treatment.

As used herein, “and/or” refers to and encompasses any and all possible combinations of one or more of the associated listed items, as well as the lack of combinations when interpreted in the alternative (or).

The term “anaerobic” means not requiring oxygen for growth. Anaerobic bacterial strains comprise bacterial strains that are obligate anaerobes (i.e., those that are harmed by the presence of oxygen); aerotolerant anaerobes, (i.e., those that cannot use oxygen for growth, but tolerate its presence); and facultative anaerobes (i.e., those that can grow without oxygen, but can use oxygen if it is present).

“Anaerobic conditions” are defined as conditions under which the oxygen concentration in the fermentation medium is too low for the microorganism to use as a terminal electron acceptor. “Anaerobic conditions” can be further defined as conditions under which no or small amounts of oxygen are added to the medium at rates of <3 mmol/L/h, preferably <2.5 mmol/L/h, more preferably <2 mmol/L/h, and most preferably <1.5 mmol/L/h. “Anaerobic conditions” means in particular completely oxygen-free (=0 mmol/L/h oxygen) or with small amounts of oxygen added to the medium at rates of e.g., <0.5 to <1 mmol/L/h. “Anaerobic metabolism” refers to a biochemical process in which oxygen is not the final acceptor of electrons contained in NADH. Anaerobic metabolism can be divided into anaerobic respiration, in which compounds other than oxygen serve as the terminal electron acceptor, and substrate level phosphorylation, in which the electrons from NADH are utilized to generate a reduced product via a fermentative pathway.

The term “carbon source” generally refers to a substrate or compound suitable for sustaining microorganism growth. Carbon sources may be in various forms, including, but not limited to polymers, carbohydrates, alcohols, acids, aldehydes, ketones, amino acids, peptides, etc. For example, these may include monosaccharides (such as glucose, fructose, xylose), oligosaccharides (i.e., sucrose, lactose), polysaccharides (i.e., starch, cellulose, hemicellulose), lignocellulosic materials, fatty acids (i.e., succinate, lactate, acetate), glycerol, etc. or a mixture thereof. The carbon source may be a product of photosynthesis, such as glucose or cellulose.

Monosaccharides used as carbon sources may be the product of hydrolysis of polysaccharides, such as acid or enzymatic hydrolysates of cellulose, starch and pectin. The term “energy source” may be used here interchangeably with carbon source since in chemoorganotrophic metabolism the carbon source is used both as an electron donor during catabolism and as a carbon source during cell growth.

Throughout this specification, unless the context requires otherwise, the words “comprise”, “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, “culturing”, “culture” and the like refer to the set of procedures used in vitro where a population of cells (or a single cell) is incubated under conditions which have been shown to support the growth or maintenance of the cells in vitro. The art recognizes a wide number of formats, media, temperature ranges, gas concentrations etc. which need to be defined in a culture system. The parameters will vary based on the format selected and the specific needs of the individual who practices the methods herein disclosed. However, it is recognized that the determination of culture parameters is routine in nature.

The terms “decrease”, “reduced”, “reduction”, “inhibit”, “suppress”, “attenuate” and the like are all used herein to mean a decrease by a statistically significant amount. In some embodiments, these terms typically mean a decrease by at least 10% as compared to a reference level (e.g., the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein “reduction”, “suppression”, and “inhibition” does not necessitate a complete inhibition or reduction as compared to a reference level. “Complete inhibition” and the like is a 100% inhibition as compared to a reference level. A decrease can be preferably down to a level accepted as within the range of normal (e.g., for an individual without a given disorder).

The terms “increased”, “increase”, enhance”, or “activate” are all used herein to mean an increase by a statistically significant amount. In some embodiments, the terms “increased”, “increase”, “enhance”, or “activate” can mean an increase of at least 10% as compared to a reference level (e.g., the absence of a given treatment or agent) and can include, for example, of at least about 10% as compared to a reference level, for example an increase of at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or up to and including a 100% increase or any increase between 10-100% as compared to a reference level or at least about a 2-fold, or at least about a 3-fold, or at least about a 3-fold, or at least about a 4-fold, or at least about a 5-fold, or at least about a 10-fold increase, or any increase between 2-fold and 10-fold or greater as compared to a reference level. In the context of a marker or symptom, an “increase” is a statistically significant increase in such level.

As used herein, the term “isolated” encompasses a bacterium or other entity or substance that has been (1) separated from at least some of the components with which it was associated when initially produced (whether in nature, such as human stool, or in an experimental setting, such as a Petri plate consisting of artificial growth medium), and/or (2) produced, prepared, purified, and/or manufactured by the hand of man. Isolated bacterial, proteins, metabolites, or combinations thereof may be separated from at least about 10%, about 20%, about 30%, about 40%, about 50%, about 60%, about 70%, about 80%, about 90%, or more of the other components with which they were initially associated. In some embodiments, isolated bacteria, proteins, metabolites, or combinations thereof are more than about 80%, about 85%, about 90%, about 91%, about 92%, about 93%, about 94%, about 95%, about 96%, about 97%, about 98%, about 99% or more than about 99% pure. As used herein, a substance is “pure” if it is substantially free of other components (such as other bacterial species). The terms “purify”, “purifying”, and “purified” refer to a bacterium or other material that has been separated from at least some of the components with which it was associated either when initially produced or generated (e.g., when in nature or in an experimental setting), or during any time after its initial production, as recognized by those skilled in the art of bacterial cultivation or of relevant skill (e.g., chemistry). A bacterium or bacterial population can be considered purified if it is isolated at or after production, such as from a material or environment containing the bacterial or bacterial population, and a purified bacterium or bacterial population can contain other material up to about 10%, about 20%, about 30%, about 40%, about 50%, about 60%, about 70%, about 80%, about 90% or above about 90% and still be considered “isolated”. In some embodiments, purified bacterial and bacterial populations are more than about 80%, about 85%, about 90%, about 91%, about 92%, about 93%, about 94%, about 95%, about 96%, about 97%, about 98%, about 99% or more than about 99% pure. In the instance of bacterial compositions provided herein, the one or more bacterial types present in the composition can be independently purified from one or more other bacteria produced and/or present in the material or environment containing the bacterial type. In some embodiments, a bacterium or population of bacteria is “isolated” if it comprises a single strain of bacteria. In some embodiments, such isolated bacteria can be admixed or administered with other isolated bacteria (e.g., in a defined consortium of isolated bacteria). Bacterial compositions and the bacterial components thereof are generally purified from residual habitat products.

The term “genome” as used herein includes the DNA comprising the genes (the coding nucleic acid sequences) and the noncoding nucleic acid sequences of a microorganism, and therefore includes introduction of the nucleic acid into, for example, the coding and noncoding DNA of the microorganism.

The term “Gram-variable” means giving a positive result and/or negative result in the Gram strain test (i.e., retaining the colour of the crystal violet staining reagent). Retention of crystal violet staining by a bacterium is linked to the thickness of the peptidoglycan layer in the bacterial cell wall. Gram-positive bacteria have a thicker peptidoglycan layer. Gram-staining is commonly used to help classify bacterial strains in the field of microbiology.

As used herein, the term “gut” is understood to refer to the human gastrointestinal tract, also known as the alimentary canal. The gut includes the mouth, pharynx, oesophagus, stomach, small intestine (duodenum, jejenum, ileum), large intestines (cecum and colon) and rectum. While the entire alimentary canal can be colonized by varying species of microbes, the majority of the gut microbiome, in terms of both numbers of species and biomass, resides in the intestines (small and large).

The terms “marker”, “biomarker” and the like, refer to any compound that can be measured as an indicator of the physiological status of a biological system. The marker may be a biomarker that comprises an amino acid sequence, a nucleic acid sequence and fragments thereof. Exemplary biomarkers include, but are not limited to cytokines, chemokines, growth and angiogenic factors, metastasis related molecules, cancer antigens, apoptosis related proteins, enzymes, proteases, adhesion molecules, cell signalling molecules and hormones. The marker may also be a sugar that, in some embodiments, may not be significantly metabolized in the biological system. The sugar may be, for example, mannitol, lactulose, sucrose, sucralose and combinations of any of the forgoing.

“Measuring” or “measurement” means assessing the presence, absence, quantity or amount (which can be an effective amount) of a given substance within a sample, including the derivation of qualitative or quantitative concentration levels of such substances, or otherwise evaluating the values or categorization of a subject's clinical parameters. Alternatively, the term “assaying,” “detecting” or “detection” may be used to refer to all measuring or measurement as described in this specification.

The term “mucosal healing” as used herein, means an improvement in one or more characteristics of that indicate an impaired mucosal layer. Such characteristics are usually determined by colonic endoscopy and include, but are not limited to, erythema, loss of vascular pattern, friability, bleeding, erosions and ulcers. In some circumstances, mucosal healing refers to a complete amelioration of detrimental effects that characterize an impaired mucosal layer. Alternatively, mucosal healing may refer to a reduction or improvement of one or more of the negative effects that characterize an impaired mucosal layer.

As used herein, the term “pharmaceutical composition” refers to the active agent in combination with a pharmaceutically acceptable carrier (e.g., a carrier commonly used in the pharmaceutical industry). The phrase “pharmaceutically acceptable” is employed herein to refer to those compounds, materials, compositions, and/or dosage forms which are, within the scope of sound medical judgement, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments, any of the aspects a pharmaceutically acceptable carrier can be a cream, emulsion, gel, liposome, nanoparticle, and/or ointment. In some embodiments of any of the aspects, a pharmaceutically acceptable carrier can be an artificial or engineered carrier (e.g., a carrier that the active ingredient would not be found to occur in or within nature).

The term “phylogenetic tree” refers to a graphical representation of the evolutionary relationships of one genetic sequence to another that is generated using defined set of phylogenetic reconstruction algorithms (e.g., parsimony, maximum likelihood, or Bayesian). Nodes in the tree represent distinct ancestral sequences and the confidence of any node is provided by a bootstrap or Bayesian posterior probability, which measures branch uncertainty.

In some embodiments, the term “strain”, refers to a terminal leaf in a phylogenetic tree and is defined by a specific genetic sequence. The specific genetic sequence may be a concatenated alignment of 120 ubiquitous single-copy proteins (Parks et al., 2018) extracted from a genome assembly using GTDB-tk (Chaumeil et al., 2020) or other tools known in the art.

The term “clade” refers to the set of members of a phylogenetic tree downstream of a stable node (bootstrap value >90%) in a phylogenetic tree. A clade is a group of related organisms representing all of the phylogenetic descendants of a common ancestor. The clade comprises a set of terminal leaves in the phylogenetic tree that is a distinct monophyletic evolutionary unit.

As used herein, “prebiotic” is understood to mean an ingredient that allows specific changes, both in the composition and/or activity in the gastrointestinal microbiota that may (or may not) confer benefits upon the host. Favoured prebiotics will be those which encourage growth of probiotic compositions or their beneficial functions, but not growth of pathogens nor genes associated with pathogenicity (e.g., toxins).

As used herein, “probiotic” is understood to mean “live microorganisms which when administered in adequate amounts confer a health benefit on the host”, as currently defined by the World Health Organization.

The term “species” is defined as a collection of closely related organisms with greater than 97% 16S ribosomal RNA (rRNA) sequence homology and greater than 70% genomic hybridization and sufficiently different from all other organisms so as to be recognized as a distinct unit. Species and other phylogenic identifications are according to the classification known to a person skilled in the art of microbiology.

As used herein, a “subject” means a human or animal. Usually the animal is a vertebrate such as a primate, rodent, domestic animal or game animal. Primates include chimpanzees, cynomolgus monkeys, spider monkeys, and macaques (e.g., Rhesus). Rodents include mice, rates, woodchucks, ferrets, rabbits, and hamsters. Domestic and game animals include cows, horses, pigs, deer, bison, buffalo, feline species (e.g., domestic cat), canine species (e.g., dog, fox, wolf), avian species (e.g., chicken, emu, ostrich), and fish (e.g., trout, catfish, and salmon). In some embodiments the subject is a mammal (e.g., a primate (e.g., a human)). The terms “individual”, “patient” and “subject” are used interchangeably herein.

Preferably the subject is a mammal. The mammal can be a human, non-human primate, mouse, rat, dog, cat, horse or cow, but is not limited to these examples. Mammals other than humans can be advantageously used as subjects that represent animal models of inflammatory and autoimmune disorders (e.g., models of gut barrier function). A subject can be male or female.

As used herein, the terms “treat”, “treatment”, “treating” and the like, refer to therapeutic treatments, wherein the object is to reverse, alleviate, ameliorate, inhibit, slow down or stop the progression or severity of a condition associated with a disease or disorder (e.g., an inflammatory or autoimmune disorder). The term “treating” includes reducing or alleviating at least one adverse effect or symptom of a condition, disease or disorder associated with an inflammatory or autoimmune disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration, or palliation of the disease state, remission (whether partial or total), and/or decreased mortality, whether detectable or undetectable. The term “treatment” of a disease also includes providing relief from the symptoms or side-effects of the disease (including palliative treatment). A treatment need not cure a disorder (i.e., complete reversal or absence of disease) to be considered effective.

In some embodiments, sequencing comprises 16S rRNA gene sequencing, which can also be referred to as “16S ribosomal RNA sequencing”, 16S rDNA sequencing” or “16S rRNA sequencing”. Sequencing of the 16S rRNA gene can be used for genetic studies as it is highly conserved between different species of bacteria, but it is not present in eukaryotic species. In addition to highly conserved regions, the 16S rRNA gene also comprises nine hypervariable regions (V1-V9) that vary between species. 16S rRNA gene sequencing typically comprises using a plurality of universal primers that bind to conserved regions of the 16S rRNA gene, PCT amplifying the bacterial 16S rRNA gene regions (including hypervariable regions), and sequencing the amplified 16S rRNA genes with a next-generation sequencing technology as described herein (see, also e.g., U.S. Pat. Nos. 5,654,418; 6,344,316; and 8,889,358; and U.S. Patent Publication Nos. 2013/157,265 and 2018/195,111, which are each incorporated by reference in their entireties).

Each embodiment described herein is to be applied mutatis mutandis to each and every embodiment unless specifically stated otherwise.

### 2. Bacterial Strains

The compositions of the invention comprise a bacterial strain of the genus Intestinicoccus. The examples demonstrate that bacteria of this genus are useful for treating or preventing diseases associated with an impaired gut barrier function. The preferred bacterial strains are of the species I. colisanans.

Intestinicoccus is a genus of bacteria in the class firmicutes. The scientific classification is as follows: bacteria (kingdom); Firmicutes (phylum); Clostridia (class); Oscillospirales (order); Acutalibacteraceae (family); Intestinicoccus (genus). Bacteria within the Intestinicoccus genus are Gram-varia ble, with a coccoid shape, and are obligate anaerobes. These criteria are important because they can inform the phylogenetic classification of bacterial strains.

The I. colisanans species has not previously been described. It has been was isolated from human stool sample, using the method described in the below examples.

The breadth of the Intestinicoccus genus and I. colisanans species may be as defined by a Genome Taxonomy Database reference tree, a taxonomic classification system as described in Parks et al., 2018.

The I. colisanans bacterium deposited under accession number V21/015887 (i.e., I. colisanans MH27-1) was tested in the Examples and is one of the preferred strains of the invention. I. colisanans strain MH27-1 was deposited with the international depositary authority National Measurement Institute (NMI, 1/153 Bertie Street, Port Melbourne, Victoria, 3207, Australia) by Microba IP Pty Ltd (388 Queen Street, Brisbane, Queensland 4000, Australia) on 6 Aug. 2021 as “Intestinicoccus colisanans MH27-1” and was assigned accession number deposited under accession number V21/015887.

An exemplary 16S rRNA sequence for the I. colisanans MH27-1 strain that was tested is set forth in SEQ ID NO: 1. Bacterial strains of the species I. colisanans may comprise a single 16S rRNA sequence within its genome, or alternatively, may comprise two or more 16S rRNA sequences within its genome (e.g., two copies, three copies, four copies, five copies, six copies, seven copies, eight copies, or more than eight copies). In some embodiments, a bacterial strain may be identified as being of the I. colisanans MH27-1 strain by determining whether the strain comprises a 16S rRNA sequence that corresponds to SEQ ID NO: 1, by any method known in the art. Chromosome sequences for strain I. colisanans MH27-1 are provided in SEQ ID NO: 3 and 4. These sequences were generated using the Illumina NovSeq6000 platform.

Bacterial strains closely related to the strains MH27-1 are also shown in the examples to be effective for treating or preventing inflammatory and autoimmune disorders, through their beneficial effects on restoring gut barrier function.

For example, the I. colisanans bacterium deposited under accession number V21/015888 (i.e., I. colisanans MH27-2) was tested in the Examples and is another one of the preferred strains of the invention. An exemplary 16S rRNA sequence for the I. colisanans MH27-2 strain that was tested is set forth in SEQ ID NO: 2. In some embodiments, a bacterial strain may be identified as being of the I. colisanans MH27-2 strain by determining whether the strain comprises a 16S rRNA sequence that corresponds to SEQ ID NO: 2, by any method known in the art. Strain I. colisanans MH27-2 was deposited with the international depositary authority National Measurement Institute (NMI, 1/153 Bertie Street, Port Melbourne, Victoria, 3207, Australia) by Microba IP Pty Ltd (388 Queen Street, Brisbane, QLD 4000, Australia) on 6 Aug. 2021 as “Intestinicoccus colisanans MH27-2” and was assigned accession number V21/015888. The genome of strain I. colisanans MH27-2 comprises a chromosome with sequences as set forth in one or both of SEQ ID NOs: 5 or 6.

In other examples, exemplary 16S rRNA sequences for the I. colisanans strains MH27-3, MH27-4, MH27-5, and MH27-6 that were tested in the examples are set forth in SEQ ID NOs: 7-10. In some embodiments, a bacterial strain may be identified as being of the I. colisanans MH27-3 strain by determining whether the strain comprises a 16S rRNA sequence that corresponds to SEQ ID NO: 7, by any method known in the art. In some embodiments, a bacterial strain may be identified as being of the I. colisanans MH27-4 strain by determining whether the strain comprises a 16S rRNA sequence that corresponds to SEQ ID NO: 8, by any method known in the art. In some embodiments, a bacterial strain may be identified as being of the I. colisanans MH27-5 strain by determining whether the strain comprises a 16S rRNA sequence that corresponds to SEQ ID NO: 9, by any method known in the art. In some embodiments, a bacterial strain may be identified as being of the I. colisanans MH27-6 strain by determining whether the strain comprises a 16S rRNA sequence that corresponds to SEQ ID NO: 10, by any method known in the art.

The genome of strain I. colisanans MH27-3 comprises a chromosome with sequences as set forth in one more of SEQ ID NOs: 11-14. The genome of strain I. colisanans MH27-4 comprises a chromosome with sequences as set forth in one more of SEQ ID NOs: 15-18. The genome of strain I. colisanans MH27-5 comprises a chromosome with sequences as set forth in one more of SEQ ID NOs: 19-22.

In certain embodiments, the bacterial strains of the invention have a 16S rRNA sequence that is at least 97.5%, 98%, 98.5%, 99%, 99.5%, 99.6%, 99.7%, 99.8%, or 99.9% identical to the 16S rRNA sequence of a bacterial strain of I. colisanans. Preferably, the bacterial strain of the invention has a 16S rRNA sequence that is at least 97.5%, 98%, 98.5%, 99%, 99.5%, 99.6%, 99.7%, 99.8%, or 99.9% identical to any one of SEQ ID NOs: 1, 2, or 7-10. In some preferred, the bacterial strain of the invention has a 16S rRNA sequence represented by any one of SEQ ID NOs: 1 2, or 7-10.

The genome of the bacterial strain may comprise the 16S rRNA sequence set forth in any one of SEQ ID NOs: 1, 2, or 7-10.

In certain embodiments, the bacterial strain of the invention has a chromosome with sequence identity to one or both of the sequences set forth in SEQ ID NO: 3 or 4. In preferred embodiments, the bacterial strain of the invention has a chromosome with at least 90% sequence identity (e.g., at least 92%, 94%, 95%, 96%, 97%, 97.5%, 98%, 98.5%, 99%, 99.5%, or 100% sequence identity) to one or both of SEQ ID NO: 3 or 4 across at least 60% (e.g., at least 65%, 70%, 75%, 80%, 85%, 95%, 96%, 97%, 98%, 99% or 100%) of SEQ ID NO: 3 or 4. For example, the bacterial strain of the invention may have a chromosome with at least 90% sequence identity to one or both of SEQ ID NO: 3 or 4 across 70% of SEQ ID NO: 3 or 4, or at least 90% sequence identity to one or both of SEQ ID NO: 3 or 4 across 80% of SEQ ID NO: 3 or 4, or at least 90% sequence identity to one or both of SEQ ID NO: 3 or 4 across 90% of SEQ ID NO: 3 or 4, or at least 90% sequence identity to one or both of SEQ ID NO: 3 or 4 across 100% of SEQ ID NO: 3 or 4, or at least 95% sequence identity to one or both of SEQ ID NO: 3 or 4 across 70% of SEQ ID NO: 3 or 4, or at least 95% sequence identity to one or both of SEQ ID NO: 3 or 4 across 80% of SEQ ID NO: 3 or 4, or at least 95% sequence identity to one or both of SEQ ID NO: 3 or 4 across 90% of SEQ ID NO: 3 or 4, or at least 95% sequence identity to one or both of SEQ ID NO: 3 or 4 across 100% of SEQ ID NO: 3 or 4, or at least 98% sequence identity to one or both of SEQ ID NO: 3 or 4 across 70% of SEQ ID NO: 3 or 4, or at least 98% sequence identity to one or both of SEQ ID NO: 3 or 4 across 80% of SEQ ID NO: 3 or 4, or at least 98% sequence identity to one or both of SEQ ID NO: 3 or 4 across 90% of SEQ ID NO: 3 or 4, or at least 98% sequence identity to one or both of SEQ ID NO: 3 or 4 across 100% of SEQ ID NO: 3 or 4. A particularly preferred strain of the invention is the I. colisanans strain deposited under accession number V21/015887. This is the exemplary MH27-1 strain tested in the DSS mouse model presented in the examples and shown to be effective for treating disease. Therefore, the invention provides a cell, such as an isolated cell, of the I. colisanans strain deposited under accession number V21/015887, or a derivative thereof. The invention also provides a composition comprising a cell of the I. colisanans strain deposited under accession number V21/015887, or a derivative thereof. The invention also provides a biologically pure culture of the I. colisanans MH27-1 strain deposited under accession number V21/015887.

In certain embodiments, the bacterial strain of the invention has a chromosome with sequence identity to SEQ ID NO: 5 or 6. In preferred embodiments, the bacterial strain of the invention has a chromosome with at least 90% sequence identity (e.g., at least 92%, 94%, 95%, 96%, 97%, 97.5%, 98%, 98.5%, 99%, 99.5%, or 100% sequence identity) to one or both of SEQ ID NO: 5 or 6 across at least 60% (e.g., at least 65%, 70%, 75%, 80%, 85%, 95%, 96%, 97%, 98%, 99% or 100%) of SEQ ID NO: 5 or 6. For example, the bacterial strain of the invention may have a chromosome with at least 90% sequence identity to one or both of SEQ ID NO: 5 or 6 across 70% of SEQ ID NO: 5 or 6, or at least 90% sequence identity to one or both of SEQ ID NO: 5 or 6 across 80% of SEQ ID NO: 5 or 6, or at least 90% sequence identity to one or both of SEQ ID NO: 5 or 6 across 90% of SEQ ID NO: 5 or 6, or at least 90% sequence identity to one or both of SEQ ID NO: or 6 across 100% of SEQ ID NO: 5 or 6, or at least 95% sequence identity to one or both of SEQ ID NO: 5 or 6 across 70% of SEQ ID NO: 5 or 6, or at least 95% sequence identity to one or both of SEQ ID NO: 5 or 6 across 80% of SEQ ID NOs: 5 or 6, or at least 95% sequence identity to one or both of SEQ ID NO: 5 or 6 across 90% of SEQ ID NO: 5 or 6, or at least 95% sequence identity to one or both of SEQ ID NO: 5 or 6 across 100% of SEQ ID NO: 5 or 6, or at least 98% sequence identity to one or both of SEQ ID NO: 5 or 6 across 70% of SEQ ID NO: 5 or 6, or at least 98% sequence identity to one or both of SEQ ID NO: or 6 across 80% of SEQ ID NO: 5 or 6, or at least 98% sequence identity to one or both of SEQ ID NO: 5 or 6 across 90% of SEQ ID NO: 5 or 6, or at least 98% sequence identity to one or both of SEQ ID NO: 5 or 6 across 100% of SEQ ID NO: 5 or 6. A particularly preferred strain of the invention is the I. colisanans strain deposited under accession number V21/015888. This is the exemplary MH27-2 strain tested in the DSS mouse model presented in the examples and shown to be effective for treating disease. Therefore, the invention provides a cell, such as an isolated cell, of the I. colisanans strain deposited under accession number V21/015888, or a derivative thereof. The invention also provides a composition comprising a cell of the I. colisanans strain deposited under accession number V21/015888, or a derivative thereof. The invention also provides a biologically pure culture of the I. colisanans MH27-2 strain deposited under accession number V21/015888.

A derivative of the strains deposited under accession number V21/015887 or V21/015888 may be a daughter strain (progeny) or a strain cultured (subcloned) from the original. A derivative of a strain of the invention may be modified, for example at the genetic level, without ablating the biological activity. In particular, a derivative strain of the invention is therapeutically active. A derivative strain will have comparable activity to the original strains from which it is derived (i.e., the strains deposited under accession numbers V21/015887 or V21/015888). In particular, a derivative strain will elicit comparable effects in at least one disease model (e.g., colitis) as shown in the Examples, which may be identified by using the culturing and administration protocols described in the Examples. A derivative of any one of the V21/015887 or V21/015888 strains will generally be a biotype of the respective V21/015887 or V21/015888 strains.

References to cells of the I. colisanans strain deposited under accession number V21/015887 include any cells that have the same safety and therapeutic efficacy characteristics as the strains deposited under any one of accession numbers V21/015887 or V21/015888, and such cells are encompassed by the invention.

### 2.1 Bacteria Biotypes

Bacterial strains that are biotypes of a bacterium deposited under accession numbers V21/015887 or V21/015888 are also expected to be effective for treating or preventing inflammatory and autoimmune disorders. A biotype is a closely related strain that has the same or very similar physiological and biochemical characteristics.

Strains that are biotypes of a bacterium deposited under accession numbers V21/015887 or V21/015888 and that are suitable for use in the invention may be identified by sequencing other nucleotide sequences for a bacterium deposited under accession numbers V21/015887 or V21/015888. For example, substantially the whole genome may be sequenced and a biotype strain of the invention may have at least 95%, 96%, 97%, 98%, 99%, 99.5% or 99.9% sequence identity across at least 80% of its whole genome (e.g., across at least 85%, 90%, 95% or 99%, or across its whole genome). Other suitable sequences for use in identifying biotype strains may include hsp60 or repetitive sequences such as BOX, ERIC, (GTG)5, or REP (Masco et al., 2003; Kim et al., 2019). Biotype strains may have sequences with at least 95%, 96%, 97%, 98%, 99%, 99.5% or 99.9% sequence identity to the corresponding sequence of a bacterium deposited under accession numbers V21/015887 or V21/015888.

Alternatively, strains that are biotypes of a bacterium deposited under accession numbers V21/015887 or V21/015888, and restriction fragment analysis and/or PCR analysis, for example by using fluorescent amplified fragment length polymorphism (FAFLP) and repetitive DNA element (rep)-PCR fingerprinting, or protein profiling, or partial 16S or 23S rRNA sequencing. In some preferred embodiments, such techniques may be used to identify other suitable I. colisanans strains.

In certain embodiments, strains that are biotypes of a bacterium deposited under accession numbers V21/015887 or V21/015888, and that are suitable for use in the invention are strains that provide the same pattern as a bacterium deposited under accession numbers V21/015887 or V21/015888 when analysed by amplified ribosomal DNA restriction analysis (ARDRA), for example when using Sau3AI restriction enzyme (for exemplary methods and guidance see, for example, Sroitkove et al., 2011). Alternatively, biotype strains are identified as strains that have the same carbohydrate fermentation patterns as a bacterium deposited under accession numbers V21/015887 or V21/015888.

In some embodiments, bacterial strains useful in the invention may be identified by routinely profiling the production and consumption of metabolites by a bacterial strain. It is predicted that the bacterial strains described above and elsewhere herein effect production of acetate. Therefore, in some embodiments, the bacterial strains of the invention induce the production in vivo of the metabolite, acetate. Additionally, in some embodiments the bacterial strains of the invention do not produce butyrate.

Other Intestinicoccus strains that are useful in the compositions and methods of the invention, such as biotypes of a bacterium deposited under accession numbers V21/015887 or V21/015888, may be identified using any appropriate method or strategy, including the assays described in the examples. For instance, strains for use in the invention may be identified by culturing in anaerobic TY or PYG media and/or administering the bacteria to the DSS-induced gut barrier function model and then assessing cytokine/chemokine levels, as described in the Examples. In particular, bacterial strains that have similar growth patterns, metabolic type and/or surface antigens to a bacterium deposited under accession numbers V21/015887 or V21/015888 may be useful in the invention. A useful strain will have comparable immunomodulatory activity to the V21/015887 or V21/015888 strain. In particular, a biotype strain will elicit comparable effects on host gut function. Furthermore, it is expected that a biotype will have a similar effect in a disease model (e.g., colitis, asthma, arthritis, multiple sclerosis and uveitis disease models) and comparable effects on cytokine/chemokine levels to the effects shown in the Examples, and which may be identified by using the culturing and administration protocols described in the Examples.

### 2.2 Bacterial Strain Viability

In preferred embodiments, the bacterial strains in the compositions of the invention are viable. In preferred embodiments, the bacterial strains in the compositions of the invention are viable and capable of partially or totally colonising the intestine. In some preferred embodiments, the bacterial strains in the compositions of the invention are live. By way of an example, the bacterial strains in the compositions of the invention have not been heat-killed. The bacteria of the invention may have immune modulatory effects that would not be exhibited by non-viable bacteria, for example because non-viable bacteria cannot produce metabolites and interact with the immune system in a different manner. The cell surface of a viable bacterium is also likely to be significantly different to a killed bacterium, in particular a heat-killed bacterium.

In some alternative embodiments, that bacteria are not viable. For example, in some embodiments the bacteria are heat-killed.

In some preferred embodiments, the bacterial strain for use in the invention is naturally-occurring. For example, the bacterial strain has been isolated from the mammalian digestive tract.

In some preferred embodiments, the bacterial strain for use in the invention has not been genetically engineered. For example, the bacterial strain has not been transformed with recombinant DNA.

### 3. Compositions

Provided herein are compositions that comprise, consist, or consist essentially of a therapeutically effective amount of a bacterial strain or strains described above and/or elsewhere herein. In some embodiments, the bacteria in the compositions may be identified by strain, species, operational taxonomic unit (OTU), whole genome sequence, 16S rRNA sequence, or other methods known in the art for defining different types of bacteria.

### 3.1 Most Recent Common Ancestor (MRCA)

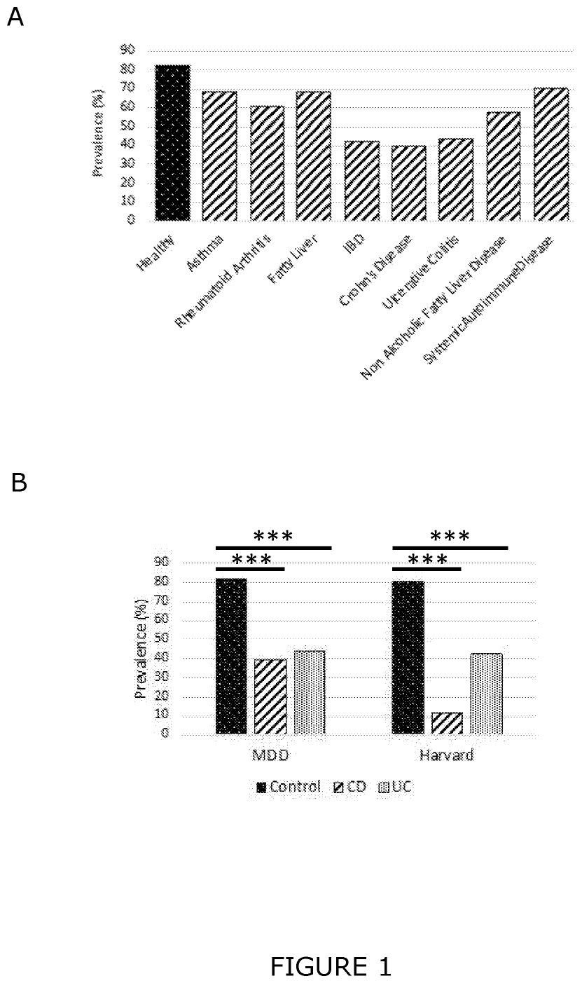

In some embodiments, the compositions comprise an effective amount of a bacterial strain that is a phylogenetic descendant of the MRCA of I. colisanans and I. sp002305575 (see, FIG. 1B). Preferably, the phylogenetic classification is as defined by the GTDB (Parks et al., 2018). In some embodiments, the phylogenetic classification is as defined in release 89 (r89) of the GTDB.

In some embodiments, determining if a bacterial strain is a descendant of a MRCA of I. colisanans and I. sp002305575 may be performed using phylogenetic grouping procedures known in the art. In some embodiments, a rooted phylogenetic tree with I. colisanans and I. sp002305575, and a third taxon of interest (e.g., a taxon to be classified) may be used, with the following analysis packages being applied: Analyses of Phylogenetics and Evolution (“ape”; https://cran.r-project.ora/web/packaaes/ape/index) and Phylogenetic Tool for Comparative Biology (“phytools”; http://cran.r-oroject.org/web/packaaes/phytoos/index.html), in order to determine whether the taxon of interest is useful for the compositions of the present invention. Both ape and phytools are packages written in the R language for use in studying molecular evolution and phylogenetics. The ape and phytools packages provide methods for phylogenetic and evolutionary analysis and their use is known to one of skill in the art. In some embodiments, the following script may be used:

library(“ape”)
library(“phytools”)
input.tree = read.tree(file=”tree_file”)
intest = c(“s_{——}Intestinicoccus colisanans”, “s_{——}Intestinicoccus
sp002305575”)
intest.node = getMRCA(input.tree, intest)
intest.tree = extract.clade(input.tree, intest.node)
print(intest.tree$tip.label)

In some embodiments, after the script is run, if the taxon of interest is in the printed list, it is a descendant of a MRCA of the two species.

In other embodiments, different phylogenetic grouping methods known in the art may be used to determine if a bacterial strain is a descendant of a MRCA of I. colisanans and (see, FIG. 1B), including methods that use different analysis packages and are based on different programming languages.

In some other embodiments, the bacterial strain is a phylogenetic descendant of the MRCA of I. colisanans and I. sp002305575, together with a pharmaceutically acceptable carrier, diluent, or excipient. Suitably, the MRCA is defined at node 23596 of the bac120 phylogenetic tree from the GTDB. In some embodiments, the phylogenetic tree is created by release 89 of the GTDB, however, any suitable subsequent release is considered to give equally as appliable results.

### 3.2 16S rRNA Sequence Identity

In some embodiments, the 16S rRNA sequence is obtained or determined for a bacterial species to be classified. This query 16S rRNA sequence is compared to 16S rRNA sequences from bacterial species already classified as members of the Intestinicoccus genus. In some embodiments, the query 16S rRNA sequence is compared to the 16S rRNA sequences set forth in SEQ ID NO: 1. In some embodiments, the query 16S rRNA sequence is compared to all known 16S rRNA sequences for bacterial species already classified as members of the Intestinicoccus genus. In other embodiments, the query 16S rRNA sequence is compared to a subset of all known 16S rRNA sequences for bacterial species already classified as members of the Intestinicoccus genus. A percent identity between the query sequence and the compared sequences is determined. If the percent identify of the query sequence is determined to be above a defined threshold, then the bacterial species to be classified is classified as member of the Intestinicoccus genus.

In some embodiments, the threshold sequence identity is 95%. In some other embodiments, the threshold sequence identity is 97.5%. In some other embodiments, the threshold sequence identity is 99.0%. In some embodiments, the threshold sequence identity is 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9% 99.0%, 99.1%, 99.2%, 99.3%, 99.4%, 99.5%, 99.6%, 99.7%, 99.8%, 99.9% or 100%.

In some embodiments, the 16S rRNA sequence is obtained or determined for a bacterial species to be classified. This query 16S rRNA sequence is compared to 16S rRNA sequences from bacterial species already classified as members of the family Acutalibacteraceae (including those set forth in any one of SEQ ID NOs: 1 2, or 7-10). In some embodiments, the query 16S rRNA sequence is compared to all known 16S rRNA sequences for bacterial species already classified as members of the family Acutalibacteraceae. In other embodiments, the query 16S rRNA sequence is compared to a subset of all known 16S rRNA sequences for bacterial species already classified as members of the family Acutalibacteraceae. A percent identity between the query sequence and the compared sequences is determined. If the percent identify of the query sequence is determined to be above a defined threshold, then the bacterial species to be classified is classified as member of the family.

In some embodiments, the threshold sequence identity is 95%. In some embodiments, the threshold sequence identity is 98.7%. In some embodiments, the threshold sequence identity is 94.8%. In some embodiments, the threshold sequence identity is 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9% 99.0%, 99.1%, 99.2%, 99.3%, 99.4%, 99.5%, 99.6%, 99.7%, 99.8%, 99.9%, or 100%.

In some embodiments, the compositions comprise an at least partially isolated bacterial strain of I. colisanans describe above and/or elsewhere herein.

In certain embodiments, the bacterial strains of the invention have a 16S rRNA sequence that is at least 97.5%, 98%, 98.5%, 99%, 99.5%, 99.6%, 99.7%, 99.8%, or 99.9% identical to the 16S rRNA sequence of a bacterial strain of I. colisanans. Preferably, the bacterial strain of the invention has a 16S rRNA sequence that is at least 97.5%, 98%, 98.5%, 99%, 99.5%, 99.6%, 99.7%, 99.8%, or 99.9% identical to either one of SEQ ID NO: 1 or 2. In some preferred embodiments, the bacterial strain of the invention has a 16S rRNA sequence represented by either one of SEQ ID NO: 1 or 2. In some other preferred embodiments, the bacterial strain of the invention has a 16S rRNA sequence represented by any one of SEQ ID NOs: 7-10.

The genome of the bacterial strain may comprise the 16S rRNA sequence set forth in any one of SEQ ID NOs: 1 2, or 7-10.

### 3.3 Genomic Sequence Identity

In certain embodiments, the bacterial strain of the invention has a chromosome with sequence identity to either one of SEQ ID NO: 3 or 4. In preferred embodiments, the bacterial strain of the invention has a chromosome with at least 90% sequence identity (e.g., at least 92%, 94%, 95%, 96%, 97%, 97.5%, 98%, 98.5%, 99%, 99.5%, or 100% sequence identity) to either one of SEQ ID NO: 3 or 4, across at least 60% (e.g., at least 65%, 70%, 75%, 80%, 85%, 95%, 96%, 97%, 98%, 99% or 100%) of SEQ ID NOs: 3 or 4. For example, the bacterial strain of the invention may have a chromosome with at least 90% sequence identity to either one of SEQ ID NO: 3 or 4 across 70% of SEQ ID NOs: 3 or 4, or at least 90% sequence identity to either one of SEQ ID NO: 3 or 4 across 80% of SEQ ID NOs: 3 or 4, or at least 90% sequence identity to either one of SEQ ID NO: 3 or 4 across 90% of SEQ ID NOs: 3 or 4, or at least 90% sequence identity to either one of SEQ ID NO: 3 or 4 across 100% of SEQ ID NOs: 3 or 4, or at least 95% sequence identity to either one of SEQ ID NO: 3 or 4 across 70% of SEQ ID NOs: 3 or 4, or at least 95% sequence identity to either one of SEQ ID NO: 3 or 4 across 80% of SEQ ID NOs: 3 or 4, or at least 95% sequence identity to either one of SEQ ID NO: 3 or 4 across 90% of SEQ ID NOs: 3 or 4, or at least 95% sequence identity to either one of SEQ ID NO: 3 or 4 across 100% of SEQ ID NOs: 3 or 4, or at least 98% sequence identity to either one of SEQ ID NO: 3 or 4 across 70% of SEQ ID NOs: 3 or 4, or at least 98% sequence identity to either one of SEQ ID NO: 3 or 4 across 80% of SEQ ID NOs: 3 or 4, or at least 98% sequence identity to either one of SEQ ID NO: 3 or 4 across 90% of SEQ ID NOs: 3 or 4, or at least 98% sequence identity to either one of SEQ ID NO: 3 or 4 across 100% of SEQ ID NOs: 3 or 4. A particularly preferred strain of the invention is the I. colisanans strain deposited under accession number V21/015887. This is the exemplary I. colisanans MH27-1 strain tested in the DSS mouse model presented in the examples and shown to be effective for treating disease. Therefore, the invention provides a cell, such as an isolated cell, of the I. colisanans strain deposited under accession number V21/015887, or a derivative thereof. The invention also provides a composition comprising a cell of the I. colisanans strain deposited under accession number V21/015887, or a derivative thereof. The invention also provides a biologically pure culture of the I. colisanans MH27-1 strain deposited under accession number V21/015887.

In certain embodiments, the bacterial strain of the invention has a chromosome with sequence identity to either one of SEQ ID NO: 5 or 6. In preferred embodiments, the bacterial strain of the invention has a chromosome with at least 90% sequence identity (e.g., at least 92%, 94%, 95%, 96%, 97%, 97.5%, 98%, 98.5%, 99%, 99.5%, or 100% sequence identity) to either one of SEQ ID NO: 5 or 6, across at least 60% (e.g., at least 65%, 70%, 75%, 80%, 85%, 95%, 96%, 97%, 98%, 99% or 100%) of SEQ ID NOs: 5 or 6. For example, the bacterial strain of the invention may have a chromosome with at least 90% sequence identity to either one of SEQ ID NO: 5 or 6 across 70% of SEQ ID NOs: 5 or 6, or at least 90% sequence identity to either one of SEQ ID NO: 5 or 6 across 80% of SEQ ID NOs: 5 or 6, or at least 90% sequence identity to either one of SEQ ID NO: 5 or 6 across 90% of SEQ ID NOs: 5 or 6, or at least 90% sequence identity to either one of SEQ ID NO: 5 or 6 across 100% of SEQ ID NOs: 5 or 6, or at least 95% sequence identity to either one of SEQ ID NO: 5 or 6 across 70% of SEQ ID NOs: 5 or 6, or at least 95% sequence identity to either one of SEQ ID NO: 5 or 6 across 80% of SEQ ID NOs: 5 or 6, or at least 95% sequence identity to either one of SEQ ID NO: 5 or 6 across 90% of SEQ ID NOs: 5 or 6, or at least 95% sequence identity to either one of SEQ ID NO: 5 or 6 across 100% of SEQ ID NOs: 5 or 6, or at least 98% sequence identity to either one of SEQ ID NO: or 6 across 70% of SEQ ID NOs: 5 or 6, or at least 98% sequence identity to either one of SEQ ID NO: 5 or 6 across 80% of SEQ ID NOs: 5 or 6, or at least 98% sequence identity to either one of SEQ ID NO: 5 or 6 across 90% of SEQ ID NOs: 5 or 6, or at least 98% sequence identity to either one of SEQ ID NO: 5 or 6 across 100% of SEQ ID NOs: 5 or 6. A particularly preferred strain of the invention is the I. colisanans strain deposited under accession number V21/015887. This is the exemplary I. colisanans MH27-1 strain tested in the DSS mouse model presented in the examples and shown to be effective for treating disease. Therefore, the invention provides a cell, such as an isolated cell, of the I. colisanans strain deposited under accession number V21/015888, or a derivative thereof. The invention also provides a composition comprising a cell of the I. colisanans strain deposited under accession number V21/015888, or a derivative thereof. The invention also provides a biologically pure culture of the I. colisanans MH27-2 strain deposited under accession number V21/015888.

### 4. Functional Features of Bacterial Strains

Gut barrier dysregulation is a key pathway leading to systemic inflammation. As demonstrated in the examples, the bacterial strains of the invention, and compositions comprising said strains, are effective at enhancing gut barrier function.

All inflammatory or autoimmune disorders mediated by gut barrier dysregulation causing systemic inflammation in the subject are applicable for treatment with the bacterial strains described above and/or elsewhere herein.

### 4.1 Gut Barrier Function

Gut barrier (also known as intestinal barrier) function regulates transport and host defense mechanisms at the mucosal interface with the outside world. Transcellular and paracellular fluxes are tightly controlled by membrane pumps, ion channels and tight junctions, adapting permeability to physiological needs.

The translocation of foreign (i.e., non-host) substances such as lipopolysaccharide (LPS) and other inflammatory compounds from the luminal side of the intestine into the circulating system is inhibited by the epithelial barrier. One of the functions of this epithelial barrier is performed by the tight junctions. Tight junctions, or zonula occludens, are the closely associated areas of two epithelial cells whose membranes join together forming a virtually impermeable barrier to fluid, thereby separating the vascular system from the lumen of the digestive tract. Disturbance at any level, but particularly bacterial translocation due to increased permeability and breakdown of oral tolerance due to compromised epithelial and T cell interaction, can result in inflammation and tissue damage. Thus, a reduction of the tight junction barrier function has been demonstrated to result in an increased translocation of undesirable substances such as LPS from intestinal lumen into the circulating system.

The present invention provides methods of restoring or improving gut barrier function in a subject, the method comprising administering to the subject a composition that comprises a bacterial strain of I. colisanans, to thereby restore or improve the gut barrier function in the subject. As used in this specification, gut barrier integrity refers to a measure of gut barrier function. High gut barrier integrity can be associated with a lack of gut or intestinal permeability, wherein a high level of gut permeability is indicative of low gut barrier integrity. In a related embodiment, the invention also provides methods of maintaining healthy or normal gut barrier function. Such methods may be used to prevent gut barrier dysregulation is subjects considered to be at high risk of gut barrier dysregulation (e.g., subjects in remission of IBD).

In some embodiments, at least one biomarker measured in a sample (and, in particular, a biological sample) is used to assess the change, in particular, an improvement, in the gut barrier integrity of a subject.

In some embodiments of the methods and uses provided in this specification, the composition comprising a bacterial strain of I. colisanans may increase or decrease the levels of one or more biomarkers of gut barrier integrity in a sample from a subject. In some embodiments, depending on the particular biomarker, either an increase or a decrease in the level of the marker is indicative of an increase in gut barrier integrity and/or a decrease in gut permeability. In some embodiments, the biomarker is selected from a cytokine, chemokine, growth factor, angiogenic factor, enzyme, protease, adhesion molecule, cell signalling molecule, hormone or sugar. In some embodiments, the biomarker comprises a cytokine. In some embodiments, the marker comprises a chemokine. In some embodiments, the marker comprises a growth factor. In some embodiments, the marker comprises an angiogenic factor. In some embodiments, the marker comprises an enzyme. In some embodiments, the marker comprises a protease. In some embodiments, the marker comprises an adhesion molecule. In some embodiments, the marker comprises a cell signalling molecule. In some embodiments, the marker comprises a hormone. In some embodiments, the marker comprises a sugar.

This specification provides assays for biomarkers of intestinal permeability. Biological samples from the subject such as blood (plasma, or serum) or tissue may be used to measure levels of any suitable biomarker including one or more of LPS, lipopolysaccharide binding protein (LPSBP), intestinal fatty acid binding protein (IFABP), Zonulin, bacterial and/or 16S rRNA, but is not limited to these markers. LPS, I-FABP and Zonulin may be measured by enzyme-linked immunosorbent assay (“ELISA”). Techniques and kits for ELISA are well known to those in the art. In some embodiments, elevated LPS, I-FABP and/or Zonulin, when compared to a control in blood, serum, saliva, urine and/or plasma, is used as an indicator of increased intestinal permeability, and, thus, lower gut barrier integrity.

LPSBP may also be measured by ELISA. In some embodiments, significant changes in LPSBP either higher or lower, when compared to a control, may be used as an indicator of increased intestinal permeability and can confirm a reduced gut barrier integrity.

In some embodiments, increases in bacterial 16S rRNA is used as an indicator of increased intestinal permeability, and, therefore, a reduction in gut barrier integrity. Bacterial 16S rRNA may be purified from blood, serum, organ tissue or urine using standard nucleic acid isolation protocols. These are, for example, commercially available. The isolated nucleic acids may be detected by qPCR amplification using primers specific for bacterial 16S rRNA sequences or amplification using primers specific for bacterial 16S rRNA and sequencing the resultant amplicons.

Tight junction proteins that are expressed by the intestinal epithelial cells and regulate intestinal permeability may also be used as biomarkers of intestinal permeability. In some embodiments, tight junction proteins are assayed to determine alterations in intestinal permeability and gut barrier integrity. In some embodiments, the proteins measured may include, but are not limited to, claudins, occludin, ZO-1, and E-cadherin (adherens junction) proteins. Other tight junction proteins may also be assayed. In some embodiments, the tight junction proteins are measured using an immunohistochemical stain. In some embodiments, the tight junction proteins are measured using ELISA.

In some embodiments, plasma citrulline is assayed to determine alterations in intestinal permeability and gut barrier integrity. A reduction in plasma citrulline levels corresponds to a loss in epithelial cell mass indicating an increase in gut barrier permeability.

In some embodiments, the method includes oral administration of an insoluble sugar such as sucralose, collection of a bodily fluid such as urine or blood after one or more defined periods of time, and measurement of the insoluble sugar contained in the bodily fluid through standard clinical analytical techniques. The insoluble sugars may include, but are not limited to, mannitol, lactulose, sucrose, sucralose and combinations of any of the foregoing.

In some embodiments, gut barrier integrity is measured using an in vitro assay. A particularly preferred in vitro assay suitable for measuring gut barrier function is by trans-epithelial electrical resistance (TEER). Such assays are well known in the field (e.g., Srinivasan, 2015; and Lea, 2015).

### 4.2 Mucosal Healing

Mucosal healing has become an important endpoint to assess the therapeutic effect in inflammatory and autoimmune disorders. The definition of full mucosal healing currently used in IBD (e.g., CD and UC) clinical trials is the “complete absence of all inflammatory and ulcerative lesions”, but this definition lacks validation and does not include mucosal improvement and grading of mucosal healing.

Mucosal healing is predominantly defined by endoscopic assessment of intestinal inflammation. In order to evaluate the presence or absence of mucosal healing on endoscopy, various endoscopic scoring systems have been developed. These indices allow for the determination of improvements of endoscopic lesions, even when the rather rigid endpoint of mucosal healing and thereby the total disappearance of all mucosal ulcerations is not met. The endoscopic component of the clinical Mayo score, introduced in 1987, is currently the most used score of the mucosal layer in clinical practice (see, Schroeder et al., 1987). It includes the variables erythema, loss of vascular pattern, friability, bleeding, erosions and ulcers, and ranges from 0 to 3. Mucosal healing is classically considered to be a score of 0 (normal mucosa) or 1 (mucosal erythema, decreased vascular pattern, mild friability) (D'Haens, 2007).

In some other embodiments, mucosal healing is determined to have occurred when the patient is determined to have an endoscopy sub-score of 0 or 1 as assessed by flexible sigmoidoscopy. In certain such embodiments, patients who experience mucosal healing are determined to have an endoscopy sub-score of 0.

Both corticosteroids and aminosalicylates have been used for decades and are among the most commonly prescribed drugs for repairing the mucous layer (e.g., in patients with UC) (Carvalho and Cotter, 2017). The mechanisms through which they reduce mucosal inflammation include controlling nuclear factor (NF)-kB expression and inflammatory cytokines (directly modulating cell migration and proliferation of epithelial cell lines). Anti-TNF drugs (e.g., infliximab, adalimumab, and golimumuab) act at several steps of mucosal injury, restricting the inflammatory infiltrate and T cell proliferation within the lamina propria (Baert, 1999), and downregulating the expression of metalloproteinases and proinflammatory molecules (Baert, 1999). They also act on the regenerative process, restoring the protective capabilities of the mucosa by reinforcing intestinal permeability and mucosal secretion, activating fibroblasts, and maintaining epithelial regeneration (Suenaert, 2002).

Other measures of assessing mucosal healing are well known in the art, including the measurement of biomarkers C-reactive protein and calprotectin. An advantage of using in vitro biomarker assays for the assessment of mucosal healing is that such assays are typically far less invasive for the subject. Histopathology is another measure of inflammation, which has been cited as being particularly informative for mucosal healing.

### 4.3 STAT3 Signalling Pathway

Cytokine pathways mediate a broad range of biological functions, including many aspects of inflammation and immunity. The Janus kinases (JAK), including JAK1, JAK2, JAK3 and tyrosine kinase 2 (TYK2), are cytoplasmic tyrosine kinases that associate with type I and type II cytokine receptors and regulate cytokine signal transduction. Cytokine engagement with cognate receptors triggers activation of receptor associated JAKs and this leads to JAK-mediated tyrosine phosphorylation of signal transducer and activator of transcription (STAT) proteins and ultimately transcriptional activation of specific gene sets (Schindler et al., 2007, J. Biol. Chem. 282: 20059-63). Cytokine receptors are typically functional as heterodimers, and as a result, more than one type of JAK kinase is usually associated with a cytokine receptor complex. The specific JAKs associated with different cytokine receptor complexes have been determined in many cases through genetic studies and corroborated by other experimental evidence.

STAT3 plays an important role in the activation of several autoimmune and inflammatory disorders, including IBD. The bacterial strains of the present invention significantly suppress IL-23-mediated STAT3 activation. Thus, the present invention provides methods of suppressing or otherwise inhibiting STAT3 signalling in a subject (i.e., IL-23-mediated STAT3 signalling), the method comprising administering to the subject a composition that comprises bacterial strain as described above and/or elsewhere herein. Accordingly, in some embodiments the bacterial strains described herein directly or indirectly suppress STAT3 activity. In some embodiments, the strain of I. colisanans produces a bioactive molecule that binds directly to a STAT3 polypeptide. In some alternative embodiments, the bacterial strain is an indirect inhibitor of STAT3 activation, for example, by binding to a molecule upstream of STAT3 in the IL-23-mediated STAT3 signalling pathway, or by binding to a molecule that regulates STAT3 activity (e.g., ubiquitination). By way of an illustrative example, the bioactive agent may directly bind or antagonize any one of IL-23, JAK2, or TYK2 in order to suppress the IL-23-mediated STAT3 signalling pathway.

I. colisanans strains reduce the activation of inflammatory cytokines such as IL-6. Chronic inflammation induced by IL-6 can ultimately lead to cell death. Therefore, the bacterial strains of the invention are particularly useful in the treatment or prevention of inflammatory or autoimmune disorders. In some embodiments, the bacterial strains are useful in the treatment of inflammatory or autoimmune disorders characterized by the enhanced activation of IL-6.

### 4.4 Th17 Inflammatory Response

Some bacterial compositions of the invention are effective for reducing the Th17 inflammatory response. In particular, treatment with the compositions described above and elsewhere herein may modulate Th17 pathway cytokines (including TNF, IL-22, IL-21, and IL-17), and result in clinical improvements in animal models of conditions mediated by the Th17 pathway. Therefore, the compositions of the invention may be useful for treating or preventing inflammatory and autoimmune disorders, and in some embodiments, diseases or conditions mediated by Th17. In particular, the compositions of the invention may be useful for reducing or preventing elevation of the Th17 inflammatory response.

Th17 cells are a subset of T helper cells that produce, among other cytokines, IL17A, IL17F, IL-21 and IL-22. Th17 cell differentiation may be driven by IL-23. These cytokines and others form important parts of the Th17 pathway, which is a well-established inflammatory signalling pathway that contributes to and underlies a number of inflammatory and autoimmune disorders (as described in, for example, Ye, 2015; Fabro, 2015; Yin, 2014; Cheluvappa, 2014; Schieck, 2014; Balato, 2014). Some diseases that are mediated by Th17 can be ameliorated or alleviated by repressing the Th17 pathway, which may be through a reduction in the differentiation of Th17 cells or a reduction in their activity or a reduction in the level of Th17 pathway cytokines. Diseases mediated by the Th17 pathway may be characterised by increased levels of cytokines produced by Th17 cells, such as IL-17A, IL-17F, IL-21, IL-22, IL-26, IL-9 (reviewed in Monteleone, 2011). Diseases mediated by the Th17 pathway may be characterised by increased expression of Th17-related genes, such as STAT3 or IL-23 receptor. Diseases mediated by the Th17 pathway may be associated with increased levels of Th17 cells.

IL-17 is a key cytokine that links T cells activation to neutrophil activation and mobilization, hence IL-17 plays a pivotal role in innate immunity. However, due to its role in neutrophil activation, can contribute to inflammatory autoimmune diseases such as inflammatory bowel disease, psoriasis, and rheumatoid arthritis. IL-17 as used herein may refer to any member of the IL-17 family, including IL-17A, IL-17B, IL-17C, IL-17D, IL-17E, and IL-17F. IL-17-mediated diseases and conditions are characterised by high expression of IL-17 and/or the accumulation, or presence of IL-17-positive cells in a tissue affected by the disease or condition. Similarly, IL-17-mediated diseases and conditions are diseases and conditions that are exacerbated by high IL-17 levels or an increase in IL-17 levels, and that are alleviated by low IL-17 levels or a reduction in IL-17 levels. The IL-17 inflammatory response may be local or systemic.

Examples of diseases and conditions that may be mediated by the Th17 pathway include (but are not limited to) inflammatory bowel disease (such as Crohn's disease and ulcerative colitis); multiple sclerosis; arthritis (such as rheumatoid arthritis, osteoarthritis, psoriatic arthritis, and juvenile idiopathic arthritis); neuromyelitis optica (Devic's disease); ankylosing spondylitis; spondyloarthritis; psoriasis; systemic lupus erythematosus; celiac disease; asthma (such as allergic asthma or neutrophilic asthma); chronic obstructive pulmonary disease (COPD); cancer (such as breast cancer, colon cancer, lung cancer or ovarian cancer); uveitis; scleritis; vasculitis; Behcet's disease; atherosclerosis; atopic dermatitis; emphysema; periodontitis; allergic rhinitis; and allograft rejection. Accordingly, in some aspects the present invention provides methods for treating or preventing one or more of these conditions or diseases, by administering a composition as described above and/or elsewhere herein. In further preferred embodiments, these conditions or diseases are mediated by the STAT3 signalling pathway. In further preferred embodiments, these conditions or diseases are mediated through the Th17 pathway.

In certain embodiments, the present invention provides methods compositions of the invention are for use in a method of reducing Th17 cell differentiation in the treatment or prevention of a disease or condition mediated by the Th17 pathway. In certain embodiments, the compositions of the invention are for use in treating or preventing an inflammatory or autoimmune disorder, wherein said treatment or prevention is achieved by reducing or preventing elevation of the Th17 inflammatory response. In certain embodiments, the compositions of the invention are for use in treating a patient with an inflammatory or autoimmune disorder, wherein the patient has elevated IL-17 levels or elevated Th17 cells or is exhibiting a Th17 inflammatory response. In certain embodiments, the patient may have been diagnosed with a chronic inflammatory or autoimmune disorder or condition, or the composition of the invention may be for use in preventing an inflammatory or autoimmune disorder or condition developing into a chronic inflammatory or autoimmune disorder or condition. In certain embodiments, the disease or condition may not be responsive to treatment with TNF inhibitors. These uses of the invention may be applied to any of the specific disease or conditions listed in the preceding paragraph.

The Th17 pathway are often associated with chronic inflammatory and autoimmune disorders, so the compositions of the invention may be particularly useful for treating or preventing chronic diseases or conditions as listed above. In certain embodiments, the compositions are for use in patients with chronic disease. In certain embodiments, the compositions are for use in preventing the development of chronic disease.

The compositions of the invention may be useful for treating diseases and conditions mediated by the Th17 pathway and for addressing the Th17 inflammatory response, so the compositions of the invention may be particularly useful for treating or preventing chronic disease, treating or preventing disease in patients that have not responded to other therapies (such as treatment with TNF inhibitors), and/or treating or preventing the tissue damage and symptoms associated with Th17 cells. For example, IL-17 is known to activate matrix destruction in cartilage and bone tissue and IL-17 has an inhibitory effect on matrix production in chondrocytes and osteoblasts, so the compositions of the invention may be useful for treating or preventing bone erosion or cartilage damage.

In certain embodiments, treatment with compositions of the invention provides a reduction or prevents an elevation in IL-17 levels, in particular IL-17A levels. In certain embodiments, treatment with compositions of the invention provides a reduction or prevents an elevation in IFN-γ or IL-6 levels. Such reduction or prevention of elevated levels of these cytokines may be useful for treating or preventing inflammatory and autoimmune disorders and conditions, in particular those mediated by the Th17 pathway.

### 4.5 Th1 Inflammatory Response

CD4⁺ T cells play an important role in inflammatory disease/disorder pathogenesis, with many subsets of CD4⁺ T cells having been identified as drivers in perpetuating chronic intestinal inflammation (see, Imam et al., 2018). For example, T helper type 1 (Th1) cells accumulate in the intestinal tract of individuals with IBD, and are directly associated with disease. Interferon-γ (IFN-γ) is the defining cytokine produced by Th1 cells. During intestinal inflammation IFN-γ in combination with TNF is proposed to drive intestinal epithelial cell β-catenin signalling and limit their differentiation and proliferation (Imam et al., 2018).

### 5. Methods of Treatment

In some embodiments, the present invention provides methods of treating or preventing an inflammatory or autoimmune disorder in a subject, the methods comprising administering to the subject a bacterial strain as described above and/or elsewhere herein.

Suitably, the inflammatory or autoimmune disorder is selected from the group comprising: an inflammatory bowel disease (such as Crohn's disease or ulcerative colitis); asthma (such as allergic asthma or neutrophilic asthma); arthritis (such as rheumatoid arthritis, osteoarthritis, psoriatic arthritis, or juvenile idiopathic arthritis); fatty liver disease (such as nonalcoholic fatty liver disease (NAFLD)); ankylosing spondylitis; psoriasis; systemic lupus erythematosus (SLE); scleroderma; Sjogren's syndrome; vasculitis; and type 1 diabetes mellitus.

### 5.1 Inflammatory Bowel Disease (IBD)

The examples demonstrate that the compositions of the invention have a beneficial restorative effect on gut barrier function and that they also have anti-inflammatory properties, and so they may be useful in the treatment of IBD. Accordingly, in some embodiments the invention provides a composition comprising a bacterial strain of the genus Intestinicoccus for use in a method of treating or preventing an inflammatory bowel disease. The inventors have identified that treatment with Intestinicoccus strains reduces severity of colitis in a mouse model of disease. Thus, the compositions of the invention may be useful in the treatment of inflammatory diseases. In some embodiments, the compositions of the invention are for use in the treatment or prevention of an IBD. In some embodiments, the invention provides methods of treating or preventing ulcerative colitis. In some embodiments, the invention provides methods of treating or preventing of Crohn's disease. In certain embodiments, the invention provides methods of treating or preventing ulcerations and/or bleeding in the treatment of an IBD, in particular in the treatment of colitis and ulcerative colitis. In preferred embodiments, the invention provides a method of treating or preventing IBD in a subject, the method comprising administering to the subject a composition comprising a bacterial strain of the species I. colisanans. In further preferred embodiments, the invention provides a method of treating or preventing colitis, (particularly ulcerative colitis) in a subject, the method comprising administering to the subject a composition comprising a bacterial strain of the species I. colisanans. In further preferred embodiments, the invention provided methods of reducing at least one side effect of colitis (particularly ulcerative colitis), including ulcerations and/or bleeding.

IBD is a complex disease that can be caused by multiple environmental and genetic factors. Factors contributing to the onset of IBD include diet, microbiota, intestinal permeability, and genetic susceptibility to increased inflammatory response to gut infection. Symptoms of inflammatory bowel disease include abdominal pain, vomiting, diarrhea, rectal bleeding, severe internal cramps/muscle spasms in the pelvic region, weight loss and anaemia. In certain embodiments, the compositions are for use in reducing one or more symptoms associated with IBD. In certain embodiments, the compositions of the invention are for use in preventing one or more symptoms of IBD.

IBD may accompany other diseases or conditions, such as cardiovascular disease, neuropsychological disorders, and metabolic syndrome. In certain embodiments, the compositions of the invention are for use in the treatment or prevention of one or more diseases or conditions that accompany IBD.

IBD is generally diagnosed by biopsy or colonoscopy. Measurements of faecal calprotectin is useful for the preliminary diagnosis of IBD. Other laboratory test for the diagnosis of IBD include, complete blood count, erythrocyte sedimentation rate, comprehensive metabolic panel, faecal occult blood test or C-reactive protein test. Typically, a combination of laboratory testing and biopsy/colonoscopy will be used to confirm diagnosis of IBD. In certain embodiments, the compositions of the invention are for use in a subject diagnosed with IBD.

In certain embodiments the IBD is Crohn's disease and/or ulcerative colitis. As broadly described above, studies have shown that several inflammatory cytokines are upregulated in the inflammatory mucosa of patients with Crohn's disease and ulcerative colitis, including but not limited to STAT3 signalling and NFκB signalling pathway-mediated cytokines (e.g., IL-17, TNF, IL-21, IL-22). Therefore, inhibition of STAT3 signalling pathway-mediated cytokine activity and/or NFκB signalling pathway-mediated cytokines may be useful in the treatment of Crohn's disease and ulcerative colitis. In certain embodiments, the compositions of the invention are for use in the treatment or prevention of Crohn's disease and/or ulcerative colitis.

Crohn's disease and ulcerative colitis are complex diseases with an array of probable causes, including genetic risk factors, diet, other lifestyle factors, such as smoking and alcohol consumption, and microbiome composition. Crohn's disease can manifest anywhere along the GI tract, whereas ulcerative colitis is typically prevalent in the large intestine and colon.

Gastrointestinal symptoms of IBD range from mild to severe and include abdominal pain, diarrhea, faecal blood, ileitis, increased bowel movements, increased flatulence, intestinal stenosis, vomiting, and perianal discomfort. The compositions of the invention may be for use in the treatment of prevention of one or more gastrointestinal symptoms of Crohn's disease and/or ulcerative colitis.

Systemic symptoms of Crohn's disease and ulcerative colitis include growth defects, such as the inability to maintain growth during puberty, decreased appetite, fever and weight loss. Extra-intestinal features of Crohn's disease include uveitis, photobia, episcleritis, gall stones, seronegative spondyloarthropathy, arthritis, enthesitis, erythema nodosum, pyoderma gangrenosum, deep venous thrombosis, pulmonary embolism, autoimmune haemolytic anaemia, clubbing and osteoporosis. Extra-intestinal features are additional conditions associated with Crohn's disease and/or ulcerative colitis that manifest outside the GI tract. Subjects with Crohn's disease also exhibit increased susceptibility to neurological complications such as seizures, strokes, myopathy, peripheral neuropathy, headache and depression. In certain embodiments, the compositions of the invention are for use in the treatment or prevention of one or more systemic symptoms of Crohn's disease and/or ulcerative colitis. In certain embodiments, the compositions of the invention are for use in the treatment or prevention of one or more extra-intestinal features of Crohn's disease and/or ulcerative colitis.

The diagnosis of Crohn's disease and ulcerative colitis usually involves carrying out multiple tests and surgical procedures, such as gastroscopy and/or colonoscopy and biopsy, typically of the ileum, radiologic tests, complete blood counts, C-reactive protein tests and erythrocyte sedimentation rates. In certain embodiments, the compositions of the invention are for use in subjects diagnosed with Crohn's disease or ulcerative colitis. In some embodiments, compositions of the invention are for use in treating a subject who has been diagnosed with Crohn's disease or ulcerative colitis.

Crohn's disease and ulcerative colitis are classified depending on the extent of the region of the GI tract affected (Gasche et al., 2000). A Crohn's disease of both the ileum and colon is classified as Ileocolic Crohn's. In some embodiments, the compositions are for use in the treatment or prevention of Ileocolic Crohn's. In some embodiments, the compositions are for use in a subject diagnosed with Ileocolic Crohn's/Crohn's ileitis is classified if only the ileum is affected. Crohn's colitis is classified if only the colon is affected. In certain embodiments, the compositions are for use in the treatment or prevention of Crohn's ileitis. In some embodiments, the compositions are for use in a subject diagnosed with Crohn's ileitis. In certain embodiments, the compositions are for use in the treatment or prevention of Crohn's colitis. In some embodiments, the compositions are for use in a subject diagnosed with Crohn's colitis.

Crohn's disease and ulcerative colitis may be treated with a number of therapeutic agents, such as corticosteroids, such as prednisone, immunosuppressive agents, such as azathioprine, or biologics, such as infliximab, adalimumab, and golimumab, vedolizumab and etrolizumab. In certain embodiments, the compositions of the invention are for use in the treatment or prevention of Crohn's disease or ulcerative colitis in combination with an additional therapeutic agent, including but not limited to those listed above. In certain embodiments, the additional therapeutic agent is for use in the treatment or prevention of Crohn's disease and/or ulcerative colitis.

### 5.2 Autoimmune Disorders

In humans, signs of intestinal inflammation are detectable before the clinical onset of many autoimmune disorders, such as type 1 diabetes (T1D) (Bosi, 2006). Similarly, augmented gut permeability appears before the development of insulitis in diabetes-prone rats in comparison with diabetes-resistant rats (Meddings, 1999; Neu, 2005). Those findings indicate that the breakage of gut barrier integrity with subsequent increased antigen trafficking and occurrence of low-grade intestinal inflammation precede the onset of T1D and are directly related to its pathogenesis, rather than secondary to diabetes-induced metabolic alterations (i.e., hyperglycemia). The gastrointestinal barrier is a fundamental gatekeeper to avoid the contact between luminal content and the human body. The barrier is composed of a mucus layer and an intestinal epithelial barrier (IEB), and both are crucial to prevent the passage of commensal bacteria, pathogens, and food antigens from the lumen into the gut tissue and systemic circulation. The IEB is a single layer of epithelial cells held together by a complex junctional system composed of tight junctional adhesion molecules (JAMs), tricellulin, and angulins whose interaction between themselves and with intracellular scaffolding proteins, i.e., zonula occludens proteins (ZOs), is fundamental to maintain tight junction integrity and control paracellular trafficking. In patients and rat models of T1D alterations of the IEB have been reported in association with gut inflammation (Meddings, 1999; Sapone, 2006). Furthermore, the importance of the gut mucus layer, an important gut barrier containing immunoregulatory molecules such as antimicrobial peptides and mucins, has recently been reported (see, Sorini et al., 2019).

In some embodiments, bacterial strains from the species I. colisanans may provide therapeutic benefits in the treatment or prevention of asthma, such as allergic asthma or neutrophilic asthma. In certain embodiments, the compositions of the invention are for use in the treatment or prevention of asthma in a subject. In certain embodiments, the invention provides a composition comprising a bacterial strain of the species I. colisanans for use in the treatment or prevention of asthma.

In some embodiments, bacterial strains from the species I. colisanans may provide therapeutic benefits in the treatment or prevention of GVHD. In certain embodiments, the compositions of the invention are for use in the treatment or prevention of GVHD in a subject. In preferred embodiments, the invention provides a composition comprising a bacterial strain of the species I. colisanans for use in the treatment or prevention of GVHD.

In some embodiments, bacterial strains from the species I. colisanans may provide therapeutic benefits in the treatment or prevention of arthritis, such as rheumatoid arthritis, osteoarthritis, psoriatic arthritis, or juvenile idiopathic arthritis. In certain embodiments, the compositions of the invention are for use in the treatment or prevention of arthritis in a subject. In certain embodiments, the invention provides a composition comprising a bacterial strain of the species I. colisanans for use in the treatment or prevention of arthritis.

In some embodiments, bacterial strains from the species I. colisanans may provide therapeutic benefits in the treatment or prevention of multiple sclerosis. In certain embodiments, the compositions of the invention are for use in the treatment or prevention of multiple sclerosis in a subject. In certain embodiments, the invention provides a composition comprising a bacterial strain of the species I. colisanans for use in the treatment or prevention of multiple sclerosis.

In some embodiments, bacterial strains from the species I. colisanans may provide therapeutic benefits in the treatment or prevention of psoriasis. In certain embodiments, the compositions of the invention are for use in the treatment or prevention of psoriasis in a subject. In certain embodiments, the invention provides a composition comprising a bacterial strain of the species I. colisanans or use in the treatment or prevention of psoriasis.

In some embodiments, bacterial strains from the species I. colisanans may provide therapeutic benefits in the treatment or prevention of systemic lupus erythematosus (SLE). In certain embodiments, the compositions of the invention are for use in the treatment or prevention of SLE in a subject. In certain embodiments, the invention provides a composition comprising a bacterial strain of the species I. colisanans for use in the treatment or prevention of SLE.

In some embodiments, bacterial strains from the species I. colisanans may provide therapeutic benefits in the treatment or prevention of allograft rejection. In certain embodiments, the compositions of the invention are for use in the treatment or prevention of allograft rejection in a subject. In certain embodiments, the invention provides a composition comprising a bacterial strain of the species I. colisanans for use in the treatment or prevention of allograft rejection.

### 6. Formulations

In some embodiments, the compositions of the invention comprises fewer than 40 different bacterial strains. In some embodiments, the composition comprises fewer than 30 different bacterial strains. In some embodiments, the composition comprises fewer than 20 different bacterial strains. In some embodiments, the composition comprises fewer than 10 different bacterial strains. In some embodiments, the composition comprises fewer than 5 different bacterial strains. In some preferred embodiments, the composition comprises fewer than 3 different bacterial strains. In some preferred embodiments, the composition comprises a single bacterial strain. In some embodiments, the composition does not comprise bacteria of the genus Clostridium.

The compositions of the invention comprise bacteria (i.e., live bacteria and/or killed bacteria). In preferred embodiments of the invention, the composition is formulated in a dried form (e.g., a freeze-dried form). The composition of the invention may comprise granules or gelatin capsules, for example hard gelatin capsules, comprising a bacterial strain of the invention. Preferably, the composition of the invention comprises lyophilised bacteria. Lyophilisation of bacteria is a well-established procedure and relevant guidance is available in, for example, references (Miyamoto-Shinohara, 2008; and Day & Stacey, 2007).

The composition of the invention may comprise a live, active bacterial culture. The examples demonstrate that cultures of the bacteria of the invention are therapeutically effective.

In some embodiments, the bacterial strain in the composition of the invention has not been inactivated, for example, has not been heat-inactivated. In some embodiments, the bacterial strain in the composition of the invention has not been killed, for example, has not been heat-killed. In some embodiments, the bacterial strain in the composition of the invention has not been attenuated, for example, has not been heat-attenuated. For example, in some embodiments, the bacterial strain in the composition of the invention has not been killed, inactivated and/or attenuated. For example, in some embodiments, the bacterial strain in the composition of the invention is live. For example, in some embodiments, the bacterial strain in the composition of the invention is viable. For example, in some embodiments, the bacterial strain in the composition of the invention is capable of partially or totally colonising the intestine. For example, in some embodiments, the bacterial strain in the composition of the invention is viable and capable of partially or totally colonising the intestine.

In some embodiments, the composition comprises a mixture of live bacterial strains and bacterial strains that have been killed. In preferred embodiments, the composition of the invention is encapsulated to enable delivery of the bacterial strain to the intestine. Encapsulation protects the composition from degradation until delivery at the target location through, for example, rupturing with chemical or physical stimuli such as pressure, enzymatic activity, or physical disintegration, which may be triggered by changes in pH. Any appropriate encapsulation method may be used. Exemplary encapsulation techniques include entrapment within a porous matrix, attachment or adsorption on solid carrier surfaces, self-aggregation by flocculation or with cross-linking agents, and mechanical containment behind a microporous membrane or a microcapsule. Guidance on encapsulation that may be useful for preparing compositions of the invention is widely available in the art (for example, in Mitropoulou, 2013; and Kailasapathy, 2002).

The composition may be administered orally and may be in the form of a tablet, capsule or powder. Encapsulated products are preferred because bacteria of the genus Intestinicoccus are obligate anaerobes.

A composition of the invention includes a therapeutically effective amount of a bacterial strain of the invention. A therapeutically effective amount of a bacterial strain is sufficient to exert a beneficial effect upon a patient. A therapeutically effective amount of a bacterial strain may be sufficient to result in delivery to and/or partial or total colonisation of the patient's intestine.

A suitable daily dose of the bacteria, for example for an adult human, may be from about 1×10³ to about 1×10¹¹ colony forming units (CFU); for example, from about 1×10⁷ to about 1×10¹⁰ CFU; in another example from about 1×10⁶ to about 1×10¹⁰ CFU; in another example from about 1×10⁷ to about 1×10¹¹ CFU; in another example from about 1×10⁸ to about 1×10¹⁰ CFU; in another example from about 1×10⁸ to about 1×10¹¹ CFU.

In certain embodiments, the dose of the bacteria is at least 10⁹ cells per day, such as at least 10¹⁰, at least 10¹¹, or at least 10¹² cells per day.

In certain embodiments, a dose of the composition may comprise the bacterial strain in an amount of from about 1×10⁶ to about 1×10¹¹ colony forming units (CFU)/g, respect to the weight of the composition. The dose may be suitable for an adult human. For example, the composition may comprise the bacterial strain from about 1×10³ to about 1×10¹¹ CFU/g; for example, from about 1×10⁷ to about 1×10¹⁰ CFU/g; in another example from about 1×10⁶ to about 1×10¹⁰ CFU/g; in another example from about 1×10⁷ to about 1×10¹¹ CFU/g; in another example from about 1×10⁸ to about 1×10¹⁰ CFU/g; in another example from about 1×10⁸ to about 1×10¹¹ CFU/g, from about 1×10⁸ to about 1×10¹⁰ CFU/g. For example, from about 1×10⁸ to about 1×10¹⁰ CFU/g. The dose may be, for example, up to or over 1 g, 3 g, 5 g, and 10 g.

In some embodiments, the compositions described above and/or elsewhere herein comprise, consist, or consist essentially of an amount of bacterial strain from about 1×10³ to about 1×10¹¹ colony forming units per gram with respect to a weight of the composition.

In some embodiments, the compositions described above and/or elsewhere herein comprise the bacterial strain at a dose of between 500 mg and 1000 mg, between 600 mg and 900 mg, between 700 mg and 800 mg, between 500 mg and 750 mg or between 750 mg and 1000 mg. In certain embodiments, the invention provides the above pharmaceutical composition, wherein the dried bacteria in the pharmaceutical composition are administered at a dose of between 500 mg and 1000 mg, between 600 mg and 900 mg, between 700 mg and 800 mg, between 500 mg and 750 mg, or between 750 mg and 1000 mg.

The composition may be formulated as a probiotic. A probiotic is defined by the FAO/WHO as a live microorganism that, when administered in adequate amounts, confers a health benefit on the host.

Typically, a probiotic, such as the composition of the invention, is optionally combined with at least one suitable prebiotic compound. A prebiotic compound is usually a non-digestible carbohydrate such as an oligosaccharide or polysaccharide, or a sugar alcohol, which is not degraded or absorbed in the upper digestive tract. Known prebiotics include commercial products such as inulin and transgalactoligosaccharides.

Other prebiotic compounds (such as vitamin C, for example), may be included as oxygen scavengers and to improve the delivery and/or partial or total colonisation and survival in vivo. Alternatively, the probiotic composition of the invention may be administered orally as a food or nutritional product, such as milk or whey based fermented dairy product, or as a pharmaceutical product.

In certain embodiments, the probiotic composition of the present invention includes a prebiotic compound in an amount of from about 1 to about 30% by weight, respect to the total weight composition (e.g., from 5 to 20% by weight). Known prebiotics include commercial products such as inulin and transgalactoligosaccharides.

In some embodiments, the prebiotic is a carbohydrate selected from the group comprising or consisting of fructooligosaccharides (or FOS), short-chain fructooligosaccharides, inulin, isomaltoligosaccharides, pectins, xylooligosaccharides (or XOS), chitosanoligosaccharides (or COS), beta-glucans, arable gum modified and resistant starches, polydextrose, tagatose, acacia fibers, carob, oats, and citrus fibers. In one aspect, the prebiotics are the short-chain fructooligosaccharides. Short-chain FOS are not digestible carbohydrates, generally obtained by the conversion of the beet sugar and including a saccharose molecule to which three glucose molecules are bonded.

The compositions of the invention may comprise pharmaceutically acceptable excipients or carriers, such as those described in Handbook of Pharmaceutical Excipients. Acceptable carriers or diluents for therapeutic use are well known in the pharmaceutical art and are described, for example, in Remington's Pharmaceutical Sciences. Examples of suitable carriers include lactose, starch, glucose, methyl cellulose, magnesium stearate, mannitol, sorbitol and the like. Examples of suitable diluents include ethanol, glycerol and water. The choice of pharmaceutical carrier, excipient or diluent can be selected with regard to the intended route of administration and standard pharmaceutical practice. The pharmaceutical compositions may comprise as, or in addition to, the carrier, excipient or diluent one or more suitable binders, lubricants, suspending agents, coating agents, and/or solubilising agents. Examples of suitable binders include starch, gelatin, natural sugars such as glucose, anhydrous lactose, free-flow lactose, 3-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. Preservatives, stabilizers, dyes and even flavouring agents may be provided in the pharmaceutical composition. Examples of preservatives include sodium benzoate, sorbic acid, cysteine and esters of 4-hydroxybenzoic acid, for example, in some embodiments the preservative is selected from sodium benzoate, sorbic acid and esters of 4-hydroxybenzoic acid. Antioxidants and suspending agents may be also used. A further example of a suitable carrier is saccharose. A further example of a suitable preservative is cysteine.

The compositions of the invention may be formulated as a food product. For example, a food product may provide nutritional benefit in addition to the therapeutic effect of the invention, such as in a nutritional supplement. Similarly, a food product may be formulated to enhance the taste of the composition of the invention or to make the composition more attractive to consume by being more similar to a common food item, rather than to a pharmaceutical composition. In certain embodiments, the composition of the invention is formulated as a milk-based product. The term “milk-based product” means any liquid or semi-solid milk-based or whey-based product having a varying fat content. The milk-based product can be, e.g., cow's milk, goat's milk, sheep's milk, skimmed milk, whole milk, milk recombined from powdered milk and whey without any processing, or a processed product, such as yoghurt, curdled milk, curd, sour milk, sour whole milk, butter milk and other sour milk products. Alternatively, the milk could be a plant-based milk, including for example, soy milk, oat milk, almond milk, coconut milk, or macadamia milk. Another important group includes milk beverages, such as whey beverages, fermented milks, condensed milks, infant or baby milks; flavoured milks, ice cream; milk-containing food such as sweets.

In some embodiments, the compositions disclosed herein comprise one or more bacterial strains of the genus Intestinicoccus and do not contain bacteria from any other species, or which comprise only de minimis or biologically irrelevant amounts of bacteria from another species. Thus, in some embodiments, the invention provides a composition comprising one or more bacterial strains of the genus Intestinicoccus (e.g., Intestinicoccus colisanans), which does not contain bacteria from any other species or which comprises only de minimis or biologically irrelevant amounts of bacteria from another species, for use in therapy.

In some embodiments, the compositions comprise one or more bacterial strains of the genus Intestinicoccus and do not contain bacteria from any other genus or comprise only de minimis or biologically irrelevant amounts of bacteria from another. In some embodiments, the compositions comprise one or more bacterial strains of the genus Intestinicoccus (e.g., Intestinicoccus colisanans) and do not contain bacteria from any other genus or comprise only de minimis or biologically irrelevant amounts of bacteria from another.

In certain embodiments, the compositions disclosed herein contain a single bacterial species and do not contain any other bacterial species. In certain embodiments, the compositions disclosed herein contain a single bacterial strain and do not contain any other bacterial strains. For example, the compositions of the invention may comprise bacteria only of a strain of I. colisanans. Such compositions may comprise only de minimis or biologically irrelevant amounts of other bacterial strains or species. Such compositions may be a culture that is substantially free from other species of organism. In some embodiments, such compositions may be a in a dried form and be substantially free from other species of organism.

In some embodiments, the invention provides a composition comprising a single bacterial strain of the genus Intestinicoccus which does not contain bacteria from any other strains or which comprises only de minimis or biologically irrelevant amounts of bacteria from another strain for use in therapy.

In some embodiments, the invention provides a composition comprising a single bacterial strain of the species Intestinicoccus colisanans (e.g., Intestinicoccus colisanans MH27-1, Intestinicoccus colisanans MH27-2, or Intestinicoccus colisanans MH27-3) and which does not contain bacteria from any other strains or which comprises only de minimis or biologically irrelevant amounts of bacteria from another strain for use in therapy.

In certain embodiments, the compositions of the invention contain a single bacterial strain or species and do not contain any other bacterial strains or species. Such compositions may comprise only de minimis or biologically irrelevant amounts of other bacterial strains or species. Such compositions may be a culture that is substantially free from other species of organism.

In certain embodiments, the compositions of the invention consist of 1, 2, 3, 4, 5, 6, 7, 8, 9, 10, 11, 12, 13, 14, 15 or 16 bacterial strains or species. In certain embodiments, the compositions consist of from 1 to 10, preferably from 1 to 5 bacterial strains or species. In some embodiments, the compositions disclosed herein comprise more than one strain from within the same species (e.g., more than 1, 2, 3, 4, 5, 6, 7, 8, 9, 10, 15, 20, 25, 30, 35, 40 or 45 strains), and, optionally, do not contain bacteria from any other species. In some embodiments, the compositions disclosed herein comprise less than 50 strains from within the same species (e.g., less than 45, 40, 35, 30, 25, 20, 15, 12, 10, 9, 8, 7, 6, 5, 4 or 3 strains), and, optionally, do not contain bacteria from any other species. In some embodiments, the compositions disclosed herein comprise 1-40, 1-30, 1-20, 1-19, 1-18, 1-15, 1-10, 1-9, 1-8, 1-7, 1-6, 1-5, 1-4, 1-3, 1-2, 2-50, 2-40, 2-30, 2-20, 2-15, 2-10, 2-5, 6-30, 6-15, 16-25, or 31-50 strains from within the same species and, optionally, do not contain bacteria from any other species. In some embodiments, the compositions disclosed herein comprise more than one species from within the same genus (e.g., more than 1, 2, 3, 4, 5, 6, 7, 8, 9, 10, 12, 15, 17, 20, 23, 25, 30, 35 or 40 species), and, optionally, do not contain bacteria from any other genus. In some embodiments, the compositions disclosed herein comprise less than 50 species from within the same genus (e.g., less than 50, 45, 40, 35, 30, 25, 20, 15, 12, 10, 8, 7, 6, 5, 4 or 3 species), and, optionally, do not contain bacteria from any other genus. In some embodiments, the compositions disclosed herein comprise 1-50, 1-40, 1-30, 1-20, 1-15, 1-10, 1-9, 1-8, 1-7, 1-6, 1-5, 1-4, 1-3, 1-2, 2-50, 2-40, 2-30, 2-20, 2-15, 2-10, 2-5, 6-30, 6-15, 16-25, or 31-50 species from within the same genus and, optionally, do not contain bacteria from any other genus. The invention comprises any combination of the foregoing.

In some embodiments, the compositions of the invention comprise more than one bacterial strain or species. For example, in some embodiments, the compositions of the invention comprise more than one strain from within the same species (e.g., more than 1, 2, 3, 4, 5, 6, 7, 8, 9, 10, 15, 20, 25, 30, 35, 40 or 45 strains), and, optionally, do not contain bacteria from any other species. In some embodiments, the compositions of the invention comprise less than 50 strains from within the same species (e.g., less than 45, 40, 35, 30, 25, 20, 15, 12, 10, 9, 8, 7, 6, 5, 4 or 3 strains), and, optionally, do not contain bacteria from any other species. In some embodiments, the compositions of the invention comprise 1-40, 1-30, 1-20, 1-19, 1-18, 1-15, 1-10, 1-9, 1-8, 1-7, 1-6, 1-5, 1-4, 1-3, 1-2, 2-50, 2-40, 2-30, 2-20, 2-15, 2-10, 2-5, 6-30, 6-15, 16-25, or 31-50 strains from within the same species and, optionally, do not contain bacteria from any other species. In some embodiments, the compositions of the invention comprise more than one species from within the same genus (e.g., more than 1, 2, 3, 4, 5, 6, 7, 8, 9, 10, 12, 15, 17, 20, 23, 25, 30, 35 or 40 species), and, optionally, do not contain bacteria from any other genus. In some embodiments, the compositions of the invention comprise less than 50 species from within the same genus (e.g., less than 50, 45, 40, 35, 30, 25, 20, 15, 12, 10, 8, 7, 6, 5, 4 or 3 species), and, optionally, do not contain bacteria from any other genus. In some embodiments, the compositions of the invention comprise 1-50, 1-40, 1-30, 1-20, 1-15, 1-10, 1-9, 1-8, 1-7, 1-6, 1-5, 1-4, 1-3, 1-2, 2-50, 2-40, 2-30, 2-20, 2-15, 2-10, 2-5, 6-30, 6-15, 16-25, or 31-50 strains from within the same genus and, optionally, do not contain bacteria from any other genus. The invention comprises any combination of the foregoing.

In certain embodiments, the pharmaceutical composition of the invention comprises between 1-50 distinct bacterial strains, such as between 1-50, 1-40, 1-30, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3 or 2 distinct bacterial strains. In certain embodiments, the pharmaceutical composition of the invention comprises between 1-50 distinct bacterial strains, such as between 1-50, 1-40, 1-30, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3 or 2 distinct bacterial strains.

In some embodiments, the composition of the invention additionally comprises a bacterial strain that has the same safety and therapeutic efficacy characteristics as a strain deposited at the NMI (Australia) under accession no. V21/015887 and/or V21/015888.

In some embodiments in which the composition of the invention comprises more than one bacterial strain, species or genus, the individual bacterial strains, species or genera may be for separate, simultaneous or sequential administration. For example, the composition may comprise all of the more than one bacterial strain, species or genera, or the bacterial strains, species or genera may be stored separately and be administered separately, simultaneously or sequentially. In some embodiments, the more than one bacterial strains, species or genera are stored separately but are mixed together prior to use.

Preferably, the compositions disclosed herein are to be administered to the gastrointestinal (GI) tract in order to enable delivery to, and/or partial or total colonisation of, the intestine with the bacterial strain of the invention. In other words, the bacteria may colonise some or all of the GI tract and such colonisation may be transient or permanent. More specifically, the phrase “total colonisation of the intestine” means that bacteria have colonised all parts of the intestine (i.e., the small intestine, large intestine and rectum). Additionally or alternatively, the term “total colonisation” means that the bacteria engraft permanently in some or all parts of the intestine.

Similarly, the phrase “partial colonisation of the intestine” means that bacteria have colonised some but not all parts of the intestine. Additionally or alternatively, the term “partial colonisation” means that the bacteria engraft transiently in some or all parts of the intestine.

The transience of engraftment of bacteria can be determined by assessing (e.g., in a fecal sample) the abundance of the bacterial strain of the invention periodically (e.g., daily or weekly) following the end of a dosing interval to determine the washout period, i.e., the period between conclusion of the dosing interval and there being no detectable levels of the bacterial strain of the invention present. In some embodiments, the washout period is 14 days or less, 12 days or less, 10 days or less, 7 days or less, 4 days or less, 3 days or less, 2 days or less, or 1 day or less.

In some embodiments, the bacteria described above or elsewhere herein engraft transiently in the large intestine.

In some embodiments, the bacterial strains of the invention are obtained from human adult faeces. In some embodiments in which the composition of the invention comprises more than one bacterial strain, all of the bacterial strains are obtained from human adult faeces or if other bacterial strains are present they are present only in de minimis amounts. The bacteria may have been cultured subsequent to being obtained from these human adult faeces and being used in a composition of the invention.

In some embodiments, the one or more Intestinicoccus bacterial strain is/are the only therapeutically active agents in a composition of the invention. In some embodiments, the bacterial strains in the composition is/are the only therapeutically active agents in a composition of the invention.

The compositions for use in accordance with the invention may or may not require marketing approval.

In certain embodiments, the invention provides the above pharmaceutical composition, wherein said bacterial strain is in a dried form. In some cases, the bacterial strain is reconstituted prior to administration. In some cases, the reconstitution is by use of a diluent described herein. In certain embodiments, the invention provides the above pharmaceutical composition, wherein said bacterial strain is spray dried. In certain embodiments, the invention provides the above pharmaceutical composition, wherein the bacterial strain is lyophilised or spray dried and wherein it is live. In certain embodiments, the invention provides the above pharmaceutical composition, wherein the bacterial strain is lyophilised or spray dried and wherein it is viable. In certain embodiments, the invention provides the above pharmaceutical composition, wherein the bacterial strain is lyophilised or spray dried and wherein it is capable of partially or totally colonising the intestine. In certain embodiments, the invention provides the above pharmaceutical composition, wherein the bacterial strain is dried (e.g., lyophilised or spray dried) and wherein it is viable and capable of partially or totally colonising the intestine. In some of the same embodiments and some alternative embodiments, the bacterial strain transiently colonises the intestine.

In some cases, the lyophilised or spray dried bacterial strain is reconstituted prior to administration. In some cases, the reconstitution is by use of a diluent described herein.

The compositions of the invention can comprise pharmaceutically acceptable excipients, diluents or carriers.

In certain embodiments, the invention provides a pharmaceutical composition comprising: a bacterial strain of the invention; and a pharmaceutically acceptable excipient, carrier or diluent; wherein the bacterial strain is in an amount sufficient to treat or prevent an inflammatory or autoimmune disorder when administered to a subject in need thereof. In some preferred embodiments, the inflammatory or autoimmune disorder is selected from the group comprising: an inflammatory bowel disease (such as Crohn's disease or ulcerative colitis); asthma (such as allergic asthma or neutrophilic asthma); arthritis (such as rheumatoid arthritis, osteoarthritis, psoriatic arthritis, or juvenile idiopathic arthritis); fatty liver disease (such as nonalcoholic fatty liver disease (NAFLD)); ankylosing spondylitis; psoriasis; systemic lupus erythematosus (SLE); scleroderma; Sjogren's syndrome; vasculitis; and type 1 diabetes mellitus.

In certain embodiments, the invention provides pharmaceutical composition comprising: a bacterial strain of the invention; and a pharmaceutically acceptable excipient, carrier or diluent; wherein the bacterial strain is in an amount sufficient to treat or prevent an inflammatory or autoimmune disorder mediated by the STAT3 signalling pathway. In preferred embodiments, said disorder is selected from the group consisting of an inflammatory bowel disease (such as Crohn's disease or ulcerative colitis); asthma (such as allergic asthma or neutrophilic asthma); arthritis (such as rheumatoid arthritis, osteoarthritis, psoriatic arthritis, or juvenile idiopathic arthritis); fatty liver disease (such as nonalcoholic fatty liver disease (NAFLD)); ankylosing spondylitis; psoriasis; systemic lupus erythematosus (SLE); scleroderma; Sjogren's syndrome; vasculitis; and type 1 diabetes mellitus.

In certain embodiments, the invention provides the above pharmaceutical composition, wherein the amount of the bacterial strain is from about 1×10³ to about 1×10¹¹ colony forming units (CFU) per gram with respect to a weight of the composition.

In certain embodiments, the invention provides the above pharmaceutical composition, wherein the composition is administered at a dose of up to or over 1 g, 3 g, 5 g or 10 g.

In certain embodiments, the invention provides the above pharmaceutical composition, wherein the composition is administered by a method selected from the group consisting of oral, rectal, subcutaneous, nasal, buccal, and sublingual.

In certain embodiments, the invention provides the above pharmaceutical composition, comprising a carrier selected from the group consisting of lactose, starch, glucose, methyl cellulose, magnesium stearate, mannitol and sorbitol.

In certain embodiments, the invention provides the above pharmaceutical composition, comprising a diluent selected from the group consisting of ethanol, glycerol and water.

In certain embodiments, the invention provides the above pharmaceutical composition, comprising an excipient selected from the group consisting of starch, gelatin, glucose, anhydrous lactose, free-flow lactose, beta-lactose, corn sweetener, acacia, tragacanth, sodium alginate, carboxymethyl cellulose, polyethylene glycol, sodium oleate, sodium stearate, magnesium stearate, sodium benzoate, sodium acetate and sodium chloride.

In certain embodiments, the invention provides the above pharmaceutical composition, further comprising at least one of a preservative, an antioxidant and a stabilizer.

In certain embodiments, the invention provides the above pharmaceutical composition, comprising a preservative selected from the group consisting of sodium benzoate, sorbic acid and esters of 4-hydroxybenzoic acid.

In certain embodiments, the invention provides the above pharmaceutical composition, wherein said bacterial strain is in a dried form (e.g., lyophilised, spray dried, fluidized bed dried, etc.).

In certain embodiments, the invention provides the above pharmaceutical composition, wherein when the composition is stored in a sealed container at about 4° C. or about 25° C. and the container is placed in an atmosphere having 50% relative humidity, at least 80% of the bacterial strain as measured in colony forming units, remains after a period of at least about: 1 month, 3 months, 6 months, 1 year, 1.5 years, 2 years, 2.5 years or 3 years.

In some embodiments, the composition of the invention is provided in a sealed container comprising a composition as described herein. In some embodiments, the sealed container is a sachet or bottle. In some embodiments, the composition of the invention is provided in a syringe comprising a composition as described herein.

The composition of the present invention may, in some embodiments, be provided as a pharmaceutical formulation. For example, the composition may be provided as a tablet or capsule. In some embodiments, the capsule is a gelatine capsule (“gel-cap”). The capsule can be a hard or a soft capsule. In some embodiments, the formulation is a soft capsule. Soft capsules are capsules which may, owing to additions of softeners, such as, for example, glycerol, sorbitol, maltitol and polyethylene glycols, present in the capsule shell, have a certain elasticity and softness. Soft capsules can be produced, for example, on the basis of gelatine or starch. Gelatine-based soft capsules are commercially available from various suppliers. Depending on the method of administration, such as, for example, orally or rectally, soft capsules can have various shapes, they can be, for example, round, oval, oblong or torpedo-shaped. Soft capsules can be produced by conventional processes, such as, for example, by the Scherer process, the Accogel process or the droplet or blowing process.

In some embodiments, the compositions disclosed herein are administered orally. Oral administration may involve swallowing, so that the compound enters the GI tract.

Pharmaceutical formulations suitable for oral administration include solid plugs, solid microparticulates, semi-solid and liquid (including multiple phases or dispersed systems) such as tablets; soft or hard capsules containing multi- or nano-particulates, liquids (e.g., aqueous solutions), emulsions or powders; lozenges (including liquid-filled); chews; gels; fast dispersing dosage forms; films; ovules; sprays; and buccal/mucoadhesive patches.

In some embodiments the pharmaceutical formulation is an enteric formulation, i.e., a gastro-resistant formulation (for example, resistant to gastric pH) that is suitable for delivery of the composition of the invention to the intestine by oral administration. Enteric formulations may be particularly useful when the bacteria or another component of the composition is acid-sensitive (e.g., prone to degradation under gastric conditions).

In some embodiments, the enteric formulation comprises an enteric coating. In some embodiments, the formulation is an enteric-coated dosage form. For example, the formulation may be an enteric-coated tablet or an enteric-coated capsule, or the like. The enteric coating may be a conventional enteric coating, for example, a conventional coating for a tablet, capsule, or the like for oral delivery. The formulation may comprise a film coating, for example, a thin film layer of an enteric polymer (e.g., an acid-insoluble polymer).

In some embodiments, the enteric formulation is intrinsically enteric, for example, gastro-resistant without the need for an enteric coating. Thus, in some embodiments, the formulation is an enteric formulation that does not comprise an enteric coating. In some embodiments, the formulation is a capsule made from a thermogelling material. In some embodiments, the thermogelling material is a cellulosic material, such as methylcellulose, hydroxymethylcellulose or hydroxypropylmethylcellulose (HPMC). In some embodiments, the capsule comprises a shell that does not contain any film forming polymer. In some embodiments, the capsule comprises a shell and the shell comprises hydroxypropylmethylcellulose and does not comprise any film-forming polymer (as described in United States Patent Publication No. US2016/0067188). In some embodiments, the formulation is an intrinsically enteric capsule (for example, VCAPS® from Capsugel).

In some embodiments, the composition is a probiotic or a medical food comprising a bacterial strain of I. colisanans. The bacteria can be administered, for instance, as a probiotic, as a capsule, tablet, caplet, pill, troche, lozenge, power, and/or granule. This strain can also be formulated as a nutraceutical, conventional food, medical food, or drug. The bacteria can also be administered as part of a fecal transplant or via suppository. In some embodiments, the composition is formulated for delivery to the gut, as described further herein, in some embodiments had the composition further comprise a prebiotic.

### 6.1 Co-Administerinp with Additional Agents

In some embodiments, the methods described herein can further comprise co-administering a second agent and/or treatment to the subject (e.g., as part of a therapy). The combination therapy, where employed, can be tailored to the particular indication. For example, where a strain of the species I. colisanans is administered to treat an inflammatory disorder (e.g., an inflammatory bowel disease), it can be administered in combination with an anti-inflammatory agent or therapy as known in the art of approved for clinical treatment of an inflammatory disorder. Other indications can be similarly treated with, for example, strains of the species I. colisanans as described herein in combination with agents known in the art or approved for the clinical treatment of those indications.

Suitable anti-inflammatory agents that could be used in the treatment of an inflammatory bowel disease include, but not necessarily limited are, the group comprising 5-aminosaliculates, corticosteroids, azathioprine, infliximab, and adalimumab.

The present invention also includes the compositions as described above, further comprising an anti-inflammatory agent. Such compositions can optionally be in the form of a single composition, or alternatively, two of more separate compositions.

### 7. Screening Methods

The invention also includes methods of identifying bacterial strains that are suitable for use in the methods of the present invention. Such methods typically include screening for a bacterial strain with a particular functional activity. Suitable assays include those described in the below examples, but any assay for measuring gut barrier function, mucosal healing, modulation of NF-κB activation, or modulation of STAT3 signalling are equally as applicable.

In some embodiments, the screening method identifies the ability of a bacterial strain of Intestinicoccus to modulate STAT3 signalling pathway. By way of an illustrative example, the invention provides a method of blocking or otherwise inhibiting the activation of STAT3 signalling in a target cell, the method comprising contacting the target cell with at least a soluble component of a bacterial cell preparation of the species Intestinicoccus colisanans, to block or otherwise inhibit the activation of STAT3 signalling in the target cell.

In some embodiments of this type, the target cell is selected from the group comprising screening a bacterial strain for a functional reporter cell (e.g., a HEK cell), an immune cell (e.g., a Th17 immune cell), an epithelial cell, and an endothelial cell.

In some embodiments, the bacterial cell preparation comprises a bacterial cell culture. Suitably, the soluble component may comprise the supernatant of the bacterial cell culture. In some embodiments of this type, the soluble component is substantially depleted of bacterial cells.

In some alternative embodiments, the bacterial cell preparation comprises a bacterial cell pellet. Preferably, the bacterial cells of the cell pellet are lysed by any means known in the art. After cell lysis, it is typical for the cell lysate soluble fraction to be separated from the insoluble fraction. The cell lysate may be subject to further processing before being during the screening assay. (e.g., diluted in a buffer), or exposed to a processing reagent.

### 8. Modes of Administration

Preferably, the compositions of the invention are to be administered to the GI tract in order to enable delivery to the intestine with the bacterial strain of the invention. Preferably, the compositions of the invention are formulated to be administered to the GI tract in order to enable delivery to the intestine with the bacterial strain of the invention. In some embodiments the compositions of the invention are formulated to be administered to the GI tract in order to enable delivery to, and partial or total colonization of, the intestine with the bacterial strain of the invention.

In certain embodiments, the compositions of the invention may be administered as a foam, as a spray or a gel.

In certain embodiments, the compositions of the invention may be administered as a suppository, such as a rectal suppository, for example in the form of a theobroma oil (cocoa butter), synthetic hard fat (e.g., SUPPOCIRE®, WITEPSOL), glycerogelatin, polyethylene glycol, or soap glycerin composition.

In certain embodiments, the compositions of the invention are administered to the GI tract via a tube, such as a nasogastric tube, orogastric tube, gastric tube, jejunostomy tube (J-tube), percutaneous endoscopic gastrostomy (PEG), or a port, such as a chest wall port that provides access to the stomach, jejunum and other suitable access ports.

The compositions of the invention may be administered once, or they may be administered sequentially as part of a treatment regimen. In certain embodiments, the compositions of the invention are to be administered daily (either once or several times). In certain embodiments, the compositions disclosed herein are administered regularly, such as daily, every two days, or weekly, for an extended period of time, such as for at least one week, two weeks, one month, two months, six months, or one year.

In some embodiments, the compositions disclosed herein are administered for 7 days, 14 days, 16 days, 21 days or 28 days or no more than 7 days, 14 days, 16 days, 21 days, or 28 days. For example, in some embodiments the compositions disclosed herein are administered for 16 days.

In certain embodiments of the invention, treatment according to the invention is accompanied by assessment of the patient's gut microbiota. Treatment may be repeated if delivery of and/or partial or total colonisation with the strain of the invention is not achieved such that efficacy is not observed, or treatment may be ceased if delivery and/or partial or total colonisation is successful, and efficacy is observed.

In certain embodiments, the composition of the invention may be administered to a pregnant animal, for example a mammal such as a human in order to prevent an inflammatory or autoimmune disorder (such as those disclosed herein) developing in her child in utero and/or after it is born.

The compositions of the invention may be administered to a patient that has been diagnosed with: a disease or condition mediated by gut barrier function dysregulation; or that has been identified as being at risk of a disease or condition mediated by gut barrier dysfunction; a disease or condition mediated by the STAT3 signalling pathway, or that has been identified as being at risk of a disease or condition mediated by the STAT3 signalling pathway; or an inflammatory or autoimmune disorder (such as those disclosed herein). The compositions may also be administered as a prophylactic measure to prevent the development of diseases or conditions mediated by the STAT3 signalling pathway in a healthy patient.

The compositions disclosed herein may be administered to a patient that has been diagnosed with an inflammatory or autoimmune disorder, in particular an inflammatory or autoimmune disorder mediated by the microbiota-gut axis, or that has been identified as being at risk of an inflammatory or autoimmune disorder, in particular an inflammatory or autoimmune disorder mediated by the microbiota-gut axis. The compositions may also be administered as a prophylactic measure to prevent the development of inflammatory or autoimmune disorders, in particular inflammatory or autoimmune disorders mediated by the microbiota-gut axis in a healthy patient.

The compositions of the invention may be administered to a patient that has been identified as having an abnormal gut microbiota. For example, the patient may have reduced or absent colonisation by bacteria of the genus Intestinicoccus, in particular I. colisanans.

The compositions of the invention may be administered as a food product, such as a nutritional supplement.

Generally, the compositions of the invention are for the prevention or treatment of human diseases, although they may be used to treat animals including monogastric mammals such as poultry, pigs, cats, dogs, horses or rabbits. The compositions of the invention may be useful for enhancing the growth and performance of animals. If administered to animals, oral gavage may be used.

In some embodiments, the subject to whom the composition is to be administered is an adult human. In some embodiments, the subject to whom the composition is to be administered is an infant human.

### 9. Culturing Methods

The bacterial strains for use in the present invention can be cultured using standard microbiology techniques as detailed in, for example, reference as taught in McSweeney, 2005.

The solid or liquid medium used for culture may, for example, be selected from TY or PYG medium.

Illustrative media formulations that are suitable for use with the present invention include those provided in Table 1.

TABLE 1
CULTURE MEDIA FORMULATIONS

| | TY | | PYG | | YG/V | | YG/P | |
|---|---|---|---|---|---|---|---|---|
| Phytone peptone | | | | | | | 20 | g |
| Tryptone | 1 | g | 20 | g | 20 | g | | |
| Vegetable | | | | | 20 | g | | |
| tryptone | | | | | | | | |
| Yeast extract | 0.25 | g | 10 | g | 10 | g | 10 | g |
| Glucose | 0.4 | g | 10 | g | 10 | g | 10 | g |
| Cellobiose | 0.1 | g | | | | | | |
| Maltose | 0.1 | g | | | | | | |
| Hemin solution | 1 | mL | 1 | mL | | | | |
| Salts 2* | 3.8 | mL | 38 | mL | 38 | mL | 38 | mL |
| Salts 3* | 3.8 | mL | 38 | mL | 38 | mL | 38 | mL |
| Sodium | 0.8 | g | 8 | g | 8 | g | 8 | g |
| bicarbonate | | | | | | | | |
| Resazurin | 0.1 | mL | 1 | mL | 1 | mL | 1 | mL |
| Cysteine | 1 | g | 1 | g | 1 | g | 1 | g |

| | | | | |
|---|---|---|---|---|
| Water | to 100 mL | to 1000 mL | to 1000 mL | To 1000 mL |

*Salts solution 2: K₂HPO₄ 6 g/L.
*Salts solution 3: KH₂PO₄ 6 g/L, (NH₄)₂SO₄ 6 g/L, NaCl 12 g/L, MgSO₄•7H 2.5 g/L, CaCl₂•2H₂O 1.6 g/L.
*Resazurin Stock 1000x (0.1%): 100 mg in 100 mL H₂O.

In order that the invention may be readily understood and put into practical effect, particular preferred embodiments will now be described by way of the following non-limiting experimental examples.

### EXAMPLES

### Intestinicoccus colisanans Association with Health and Disease

Inflammatory bowel disease is characterized by structure-function changes to the microbiome with a significant reduction in both the prevalence and abundance of select gut bacteria in the IBD gut when compared to the healthy gut. Several studies have shown that these bacteria may modulate IBD pathogenesis (Mallone et al., 2011; and Sokol et al., 2008) however a key obstacle to using these bacteria to develop new therapeutics has been that low resolution 16S rRNA based profiling do not provide sufficient resolution to accurately discriminate against health and IBD associated strains at a low taxonomic level (i.e., genus, species, strain).

The inventors used the Microba Disease Database (MDD), which contains high resolution faecal gut metagenomic data and associated host metadata for 6,020 adults, to study the prevalence of I. colisanans in inflammatory and autoimmune diseases. Metagenomic sequence reads were analysed using the Microba Community Profiler (MCP) (Parks et al., 2021).

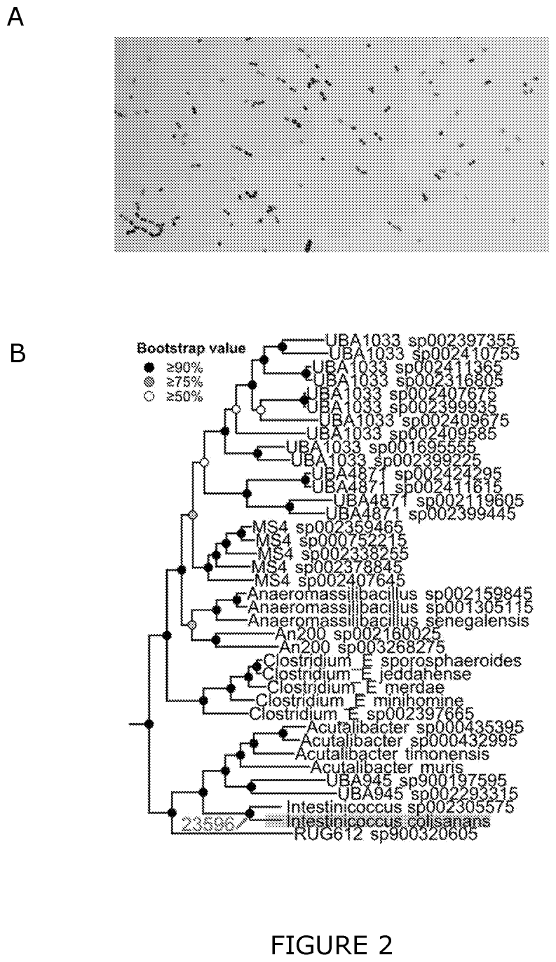

I. colisanans was considered to be prevalent in healthy humans but were less detected in inflammatory and autoimmune diseases (FIG. 1A). The strongest effect was observed for IBD, including both major subtypes ulcerative colitis and Crohn's disease (FIG. 2A, and Table 2). This observation was replicated in an independent IBD cohort previously published by Harvard ((Franzosa et al., 2018), FIG. 1B, and Table 3).

TABLE 2
ASSOCIATION STATISTICS BETWEEN I. COLISINANS
AND INFLAMMATORY DISEASES

| | P | | |
|---|---|---|---|
| | (Fisher's | | |
| | exact | | Prevalence |
| Condition | test) | FDR | (%) |
| Healthy | | | 82.1 |
| Asthma | 0.00037 | 0.00243 | 68.3 |
| Rheumatoid | 2.0 × 10⁻⁴ | 0.00719 | 60.9 |
| arthritis | | | |
| Fatty liver | 0.019 | 0.0904 | 68.7 |
| IBD | 8.1 × 10−¹⁶ | 2.99 × 10⁻¹³ | 42.6 |
| Crohn's disease | 1.1 × 10⁻⁸ | 7.47 × 10⁻⁷ | 39.5 |
| Ulcerative colitis | 1.6 × 10⁻¹¹ | 2.36 × 10⁻⁹ | 43.9 |
| Non-alcohol fatty | 0.00071 | 0.0126 | 57.5 |
| liver disease | | | |
| Systemic | 0.007 | 0.0319 | 70.2 |
| autoimmune | | | |
| disease | | | |

TABLE 3
ASSOCIATION STATISTICS BETWEEN
I. COLISANANS AND IBD

| | | | | | | | P |
|---|---|---|---|---|---|---|---|
| | Prevalence (%) | | | | | | Cochran- |
| | Con- | | | | | | Mantel- |
| | trol | CD | UC | Control | CD | UC | Haenszel |
| Taxon | MDD | MDD | MDD | Harvard | Harvard | Harvard | test |
| I. | 82.1 | 39.5 | 43.9 | 80.4 | 11.9 | 42.6 | 4.0 × |
| colisanans | | | | | | | 10⁻²⁵ |

Isolation and Genome-Scale Analysis of I. colisanans

To better understand the role I. colisanans in health and the pathogenesis of IBD we isolated two new isolates, termed I. colisanans MH27-1 and MH27-2 from healthy human donors by generating dilution-to-extinction enrichments and then plating for single colonies. I. colisanans MH27-1 and MH27-2 grew on TY and PYG based media and were typically observed as a Gram-variable staining coccus like cells (FIG. 2A).

Phylogenetic reconstruction of the Intestinicoccus genus using high-quality GenBank and RefSeq genomes from the NCBI and I. colisanans MH27-1 and MH27-2 revealed the isolates placed with high confidence within the I. colisanans clade, close to several uncultured species (FIG. 2B), and sister to the Acutalibacter genus. I. colisanans MH27-1 and I. colisanans MH27-2 are sugar fermenters, and are predicted to utilize a wide range of carbohydrate sources, notably starch, galactose, and mannose. Complete, or near complete biosynthetic pathways were identified for most amino acids except tryptophan, glycine, histidine, phenylalanine and tyrosine. Several pathways for the uptake and fermentation of amino acids (Arg, Asp, Cys, Met, Glu, Gln, Met, Ser, and Thr) were also identified, but are likely not primary sources of energy. Metabolic modelling of the I. colisanans MH27-1 and I. colisanans MH-27-2 isolates revealed that when fed with glucose and other hexose sugars, the primary by-product of fermentation was acetate. AntiSmash identified two RiPP sactipeptides BGCs in both strains with no close homology to BGCs in public databases. DeepBGC identified the same sactipeptides RiPP and a further seven putative BGCs with >1 coding region and >0.75 deepBGC score.

I. colisanans Enhances Gut Barrier Function

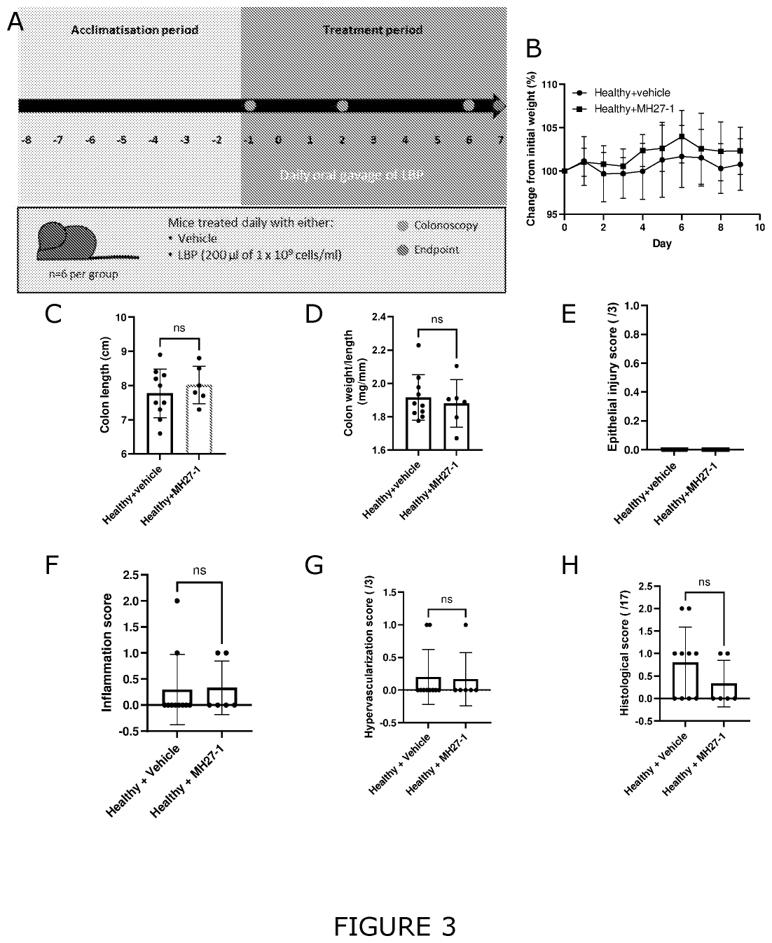

To assess the role of I. colisanans in the healthy gut, naive C57Bl/6 SPF mice were treated for 8 days with I. colisanans MH27-1 (FIG. 3A). During this treatment period, we did not observe any morbidity or change in general appearance, behaviour, posture, mobility and neurological behaviour. Similarly, there was no significant change in body weight in I. colisanans MH27-1 treated animals relative to the vehicle control and colon length and weight/length ratio were also unaffected (FIGS. 3B-D). I. colisanans MH27-1 did not result in any significant histological changes in the colon when compared to the vehicle as determined by assessing epithelial injury, inflammation and hypervascularization alone, or as a combined histopathological score (FIG. 3E-H).

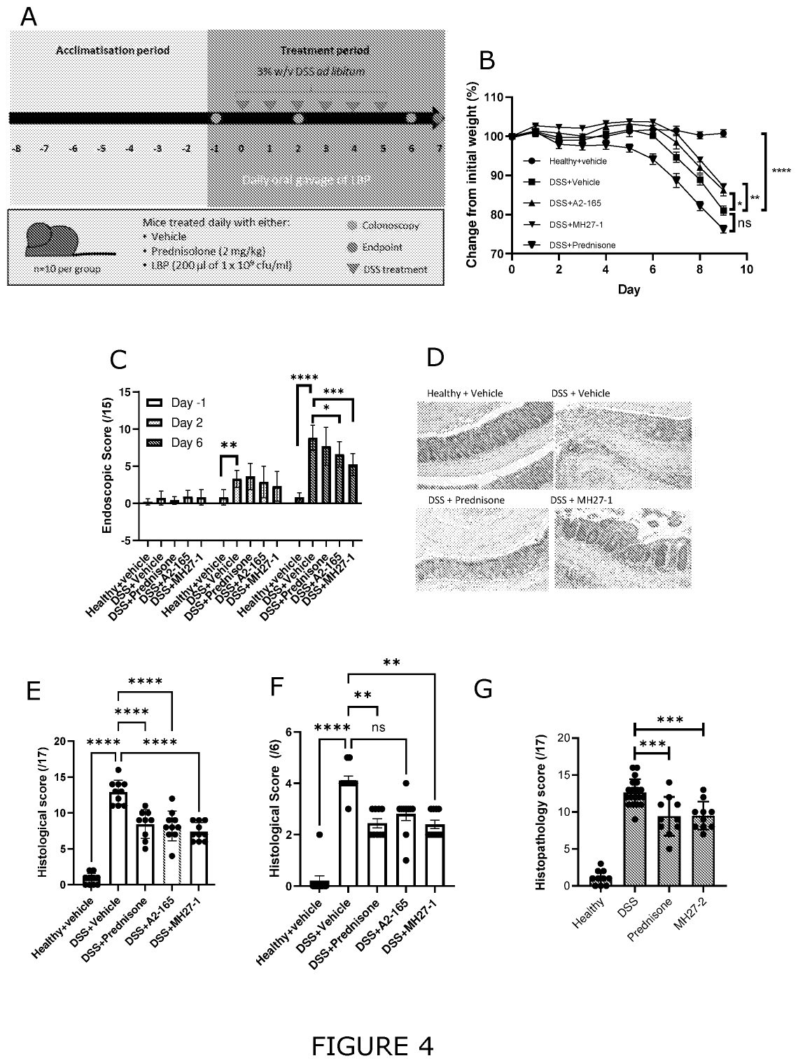

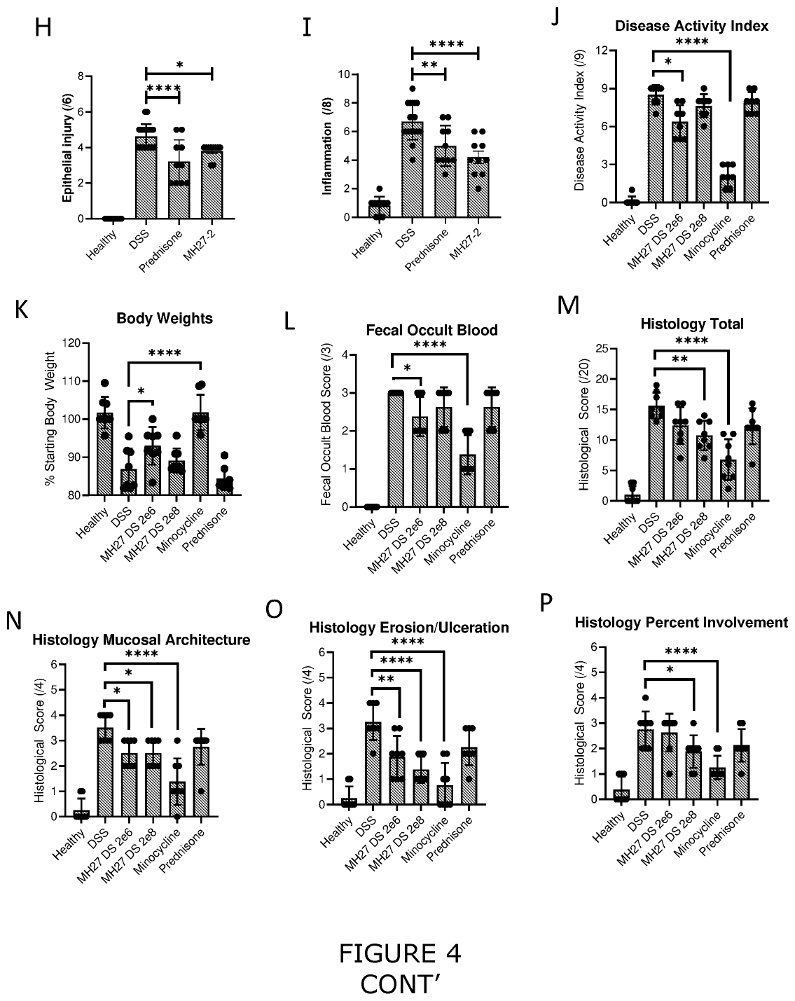

The present inventors hypothesised that the big-data approach could be used to identify novel candidate live biotherapeutics for inflammatory bowel disease. We therefore tested the therapeutic efficacy of I. colisanans MH27-1 in an acute model of DSS induced murine colitis (FIG. 4A) as this is a well-documented model of epithelial injury and repair. DSS treatment resulted in significant disease activity relative to the vehicle control. There was a significant reduction in body weight (FIG. 4B) which has been shown to be an accurate and reliable indicator of colitis (Britto, 2019). As expected, prednisone exacerbated the DSS induced weight loss (Yamamoto, 2013), however DSS induced weight loss was ameliorated by treatment with I. colisanans MH27-1 or F. prausnitzii A2-165 (FIG. 4B). Endoscopic analysis revealed a progressive increase in disease activity in all treatment groups with F. prausnitzii A2-165 and I. colisanans MH27-1 but not prednisone resulting in lower disease activity at day 6 relative to the vehicle treatment group (FIG. 4C).

Histological analysis of DSS treated mice revealed significant gut damage characterised by crypt loss, epithelial erosion and ulceration. Notably, treatment with I. colisanans MH27-1 resulted in significant improvement in pathology characterised by crypt re-formation and re-epithelisation (FIG. 4D) as evidenced by improvements in histopathological healing (FIG. 4E) and epithelial injury (FIG. 4F). As expected, prednisone and F. prausnitzii A2-165 also resulted in a significant improvement in disease pathology.

The therapeutic efficacy of I. colisanans MH27-2 in an acute mouse model of DSS colitis was examined, with prednisone acting as a positive control drug. DSS treatment resulted in significant colitis, as evidenced by histopathological scoring of colon tissue damage and inflammation.

Histological evidence of colitis was ameliorated by daily treatment with I. colisanans MH27-2 or the positive control drug prednisone. Specifically, the total histological score of DSS-treated mice given daily treatment with I. colisanans MH27-2 was significantly lower compared to DSS control mice (FIG. 4G). This effect was also observed for each of the histological sub-scores, demonstrating that treatment with I. colisanans MH27-2 reduced both epithelial damage (FIG. 4H) and inflammation (FIG. 4I) in DSS-treated mice.

The therapeutic efficacy of I. colisanans MH27-2 drug substance (DS) in an acute mouse model of DSS colitis was examined, with minocycline and prednisone as positive controls. DSS treatment resulted in significant colitis, as evidenced by both in vivo colitis measurements (disease activity index, body weight, fecal occult blood) and histopathological damage to the colon tissue (histology scores and sub-scores).

Disease activity index was significantly ameliorated (p<0.05) by daily treatment with I. colisanans MH27-2 DS at a dose of 2×10\hat{ }6 cells per day (FIG. 4J), or by the positive control minocycline. Specifically, the disease activity index sub-scores of body weight change (FIG. 4K) and fecal occult blood (FIG. 4L) were improved by daily treatment with I. colisanans DS or minocycline. Total histopathological scores were significantly improved in DSS-treated mice given daily treatment with either minocycline or I. colisanans MH27-2 DS at a dose of 2×10\hat{ }8 cells per day (FIG. 4M). This therapeutic effect was characterized by improvements in mucosal architecture scores (FIG. 4N), erosion/ulceration scores (FIG. 4O) and percent involvement scores (FIG. 4P).

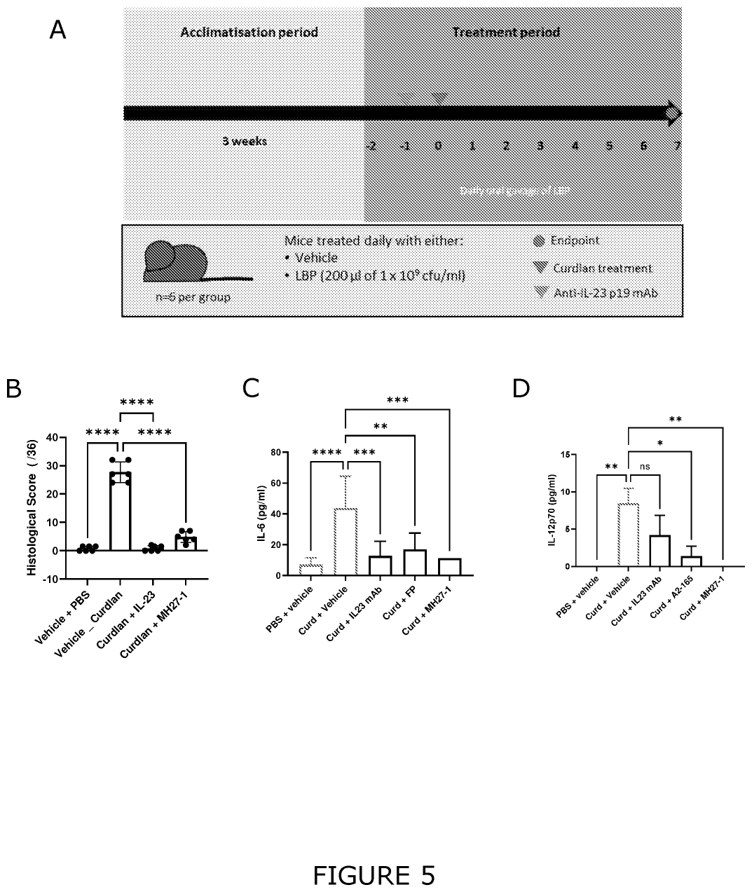

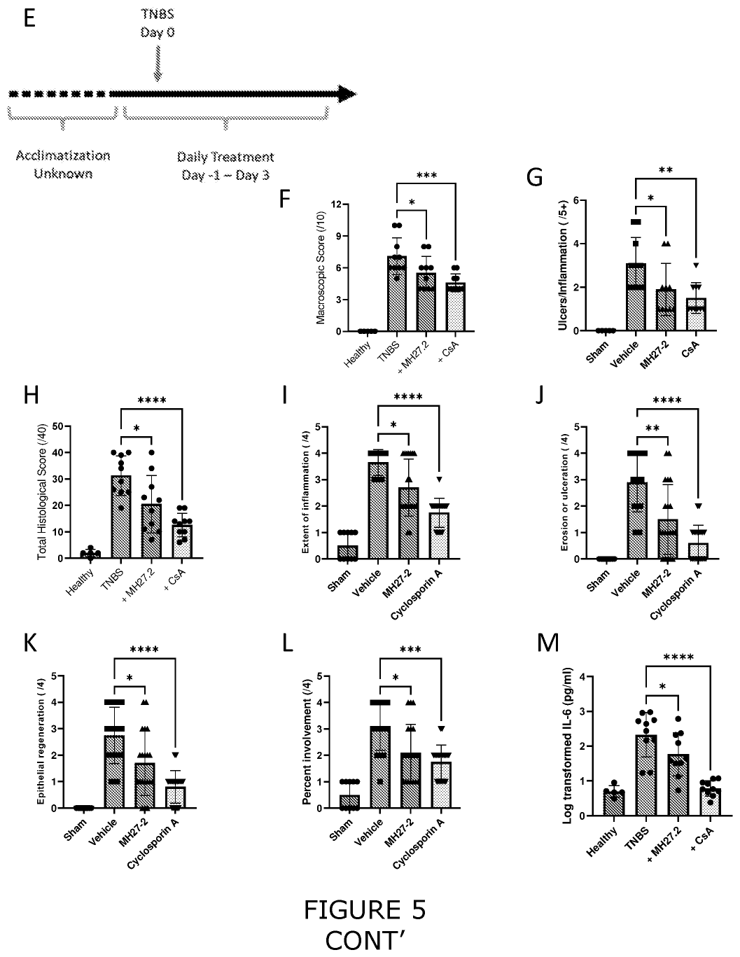

The IL-23-Th17 cell immune axis is central to the pathogenesis of inflammatory bowel disease and is a validated therapeutic target (Friedrich, 2019). As the DSS model of murine colitis is largely underpinned by Th1 polarised immunity (Yang, 2017) we also tested the therapeutic efficacy of I. colisanans MH27-1 in the murine SKG model. SKG mice carry a mutation in the ZAP-70 gene and develop IL-23 driven Crohn's like ileitis and autoimmune inflammatory arthritis following disease initiation with curdlan treatment (FIG. 5A) (see, Benham, 2014). Histological analysis of vehicle treated mice revealed significant gut damage characterised by infiltration of inflammatory cells and granuloma formation 7 days after curdlan treatment. As expected, treatment with the anti-IL-23 monoclonal antibody resulted in a significant reduction in histological damage. Treatment with I. colisanans MH27-1 also resulted in significant improvement in pathology as evidenced by improvements in the histopathological score (FIG. 5B). Similarly, treatment with I. colisanans MH27-1 also resulted in significant reduction in the production of cytokines central to disease pathogenesis including IL-6 and IL-12 (IL-12p70) (FIG. 5C, D).

The therapeutic efficacy of I. colisanans MH27-2 in an acute murine model of TNBS-induced colitis was also examined, with cyclosporine A as a positive control (FIG. 5E). The TNBS model is widely used as it shares immunopathologies with Crohn's disease. TNBS treatment resulted in significant macroscopic damage that was ameliorated by treatment with cyclosporine A or I. colisanans MH27-2 (FIG. 5F). Notably, I. colisanans MH27-2 showed significant (p<0.05) improvement in the ulcers/inflammation score (FIG. 5G). TNBS treatment resulted in significant histological damage that was ameliorated by treatment with cyclosporine A or I. colisanans MH27-2 (FIG. 5H). The colitis lesions including diffuse mucosal architectural abnormalities, ulceration, crypt dilation, aberrant crypts, crypt loss, distortion of mucosal glands, goblet cell loss and focal to multifocal inflammatory cell infiltration were less pronounced in I. colisanans MH27-2 compared to the vehicle group and the lesion scores were significantly (p<0.05) reduced in the treatment groups as reflected by improvements in the individual extent of inflammation, erosion or ulceration, epithelial regeneration and percent involvement scores (FIG. 5I-L). Treatment with I. colisanans MH27-2 resulted in significant reduction in IL-6 concentration when compared to the vehicle treated TNBS control (FIG. 5M).

Taken together, these data revealed that I. colisanans MH27-1 did not cause any adverse effects in DSS treated or naïve mice, and that I. colisanans MH27-1 and 2 promoted mucosal healing in DSS or TNBS treated C57Bl/6, and SKG mice.

I. colisanans Suppresses IL-6 Mediated Activation of STAT3.

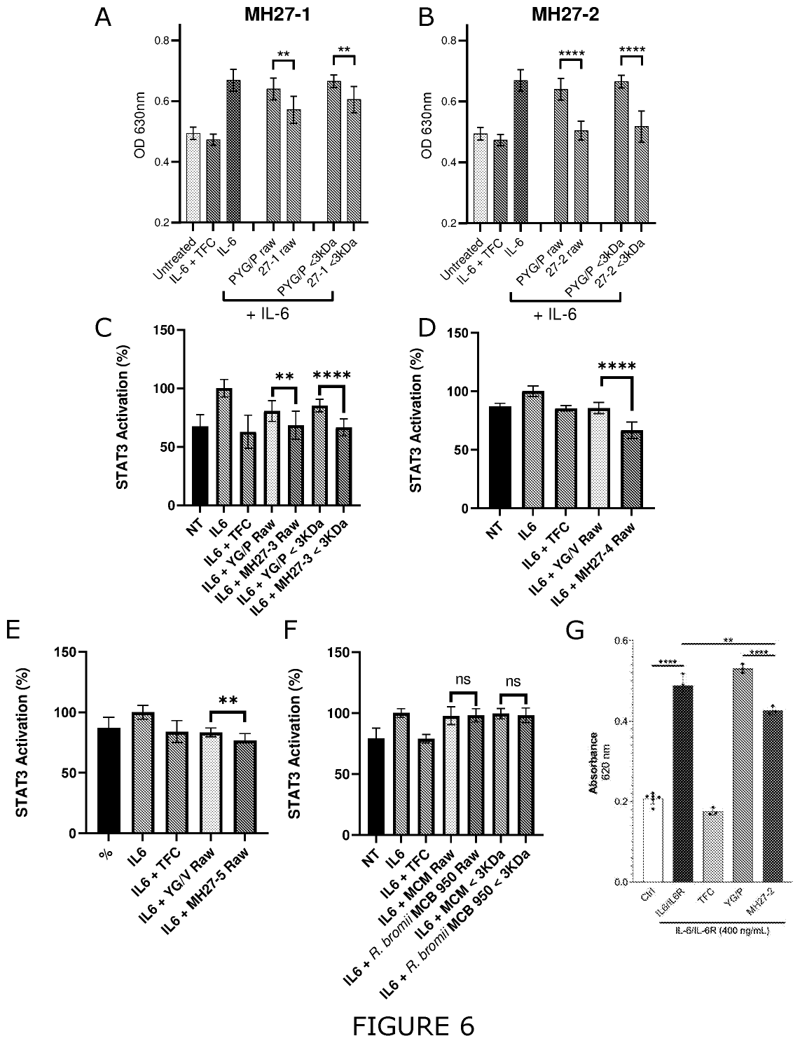

Increased levels of the pro-inflammatory cytokine IL-6 and signalling via its receptor IL-6R is associated with IBD pathogenesis. Notably, IL-6 contributes to chronic inflammation in the gut due to its pro-inflammatory and anti-apoptotic effects on immune cells. These effects are mediated by IL-6 receptor binding which causes JAK kinase activation and STAT3 dimerization in combination with activation of MAPK/ERK and other downstream kinases. Here, we examined whether I. colisanans MH27-1 and MH27-2 can suppress IL-6 classical and trans signalling mediated activation of STAT3 using the HEK-Blue™ IL-6 reporter cell line. The HEK-Blue™ IL-6 reporter cell line constitutively expresses the human IL-6 receptor and binding of IL-6 to the receptor triggers expression of a STAT3-responsive SEAP reporter. Tofacitinib was used as a control, and as expected, IL-6 and IL-6/IL-6R mediated activation of STAT3 was fully prevented by tofacitinib (FIG. 6). It is demonstrated that I. colisanans MH27-1, MH27-2 and MH27-3 raw culture supernatant and its <3 kDa filtered fraction significantly suppressed SEAP reporter activity (FIG. 6AC) with I. colisanans MH27-4 and MH27-5 raw culture supernatant also suppressing SEAP reporter activity relative to the medium (Figure DE). In contrast, R. bromii MCB950 raw culture supernatant and its <3 kDa filtered fraction did not significantly suppress SEAP reporter activity (FIG. 6F). Additionally, I. colisanans MH27-2 suppressed IL-6 trans signalling mediated activation of STAT3 (FIG. 6G).

I. colisanans Promotes the Migration of Human Intestinal Epithelial Cells.

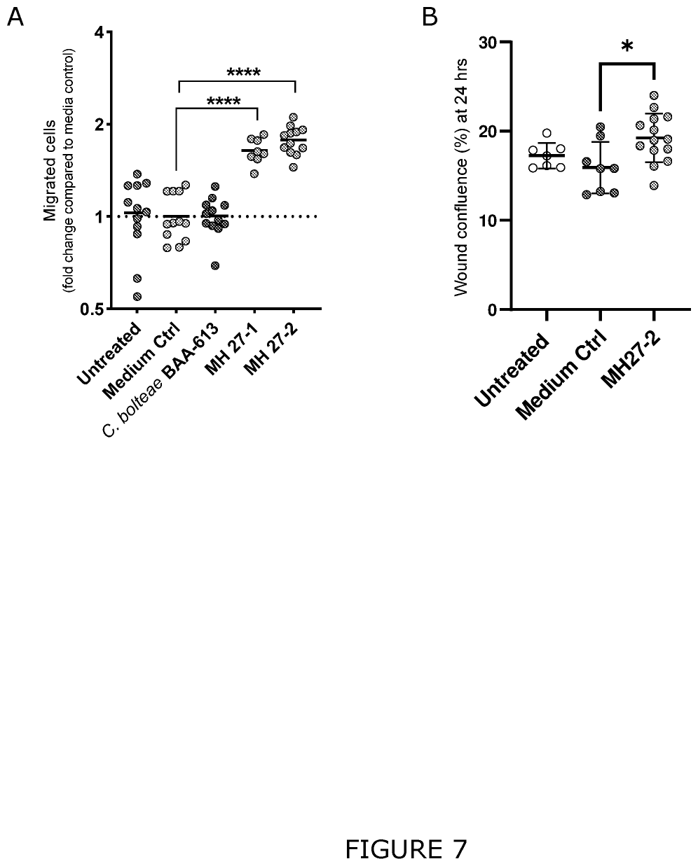

Damage of the intestinal barrier commonly occurs in IBD. The rapid migration of intestinal epithelial cells is a crucial component of the wound healing process to re-establish homeostasis. To determine whether I. colisanans MH27-1 or MH27-2 can affect the motility of intestinal epithelial cells, a Transwell® migration assay was employed. HCT116 cells were seeded apically in a Transwell® chamber and the ability of I. colisanans extract to promote migration to the basolateral side of the chamber was assessed. Untreated cells had a basal level of migration to the basolateral side of the membrane, and this was unaffected by treatment with TY medium or the gut bacterium Clostridium bolteae BAA-613 (FIG. 7A). I. colisanans MH27-1 and MH27-2 significantly promoted the migration of HCT116 cells to the basolateral side (FIG. 7A).

The pro-migratory effects of I. colisanans were further confirmed using the IncuCyte scratch wound assay. After induction of a scratch wound, HCT116 cells showed an accelerated rate of wound closure in the presence of extract from I. colisanans MH27-2 compared to the control cells treated with medium (FIG. 7B).

I. colisanans MH27 Suppresses IL-23 Mediated Activation of STAT3.

The present inventors next sought to investigate whether bacterial products naturally produced by the gut bacterium I. colisanans MH27, can modulate IL-23 mediated activation of STAT3. The JAK-STAT immune axis is a recognised and validated drug target for IBD (Salas et al., 2020) and other diseases (McLornan et al., 2021) with JAK-STAT pathway signalling targeted by several approved IBD therapeutics including anti-IL-23 antibodies (e.g., Ustekinumab) and small molecule inhibitors (e.g., tofacitinib) (Hu et al., 2021). As STAT3 suppression can reduce damaging inflammation, I. colisanans MH27 and/or its bioactives could potentially be used as novel immunoregulatory therapies for IBD and other diseases. The present inventors therefore examined whether I. colisanans MH27 can suppress IL-23-mediated activation of STAT3 using the HEK-Blue™ IL-23 reporter cell line. The HEK-Blue™ IL-23 reporter cell line constitutively expresses the human IL-23 receptor and binding of IL-23 to the receptor triggers expression of a STAT3-responsive SEAP reporter. The pharmacological JAK-STAT inhibitor, tofacitinib, was used as a control.

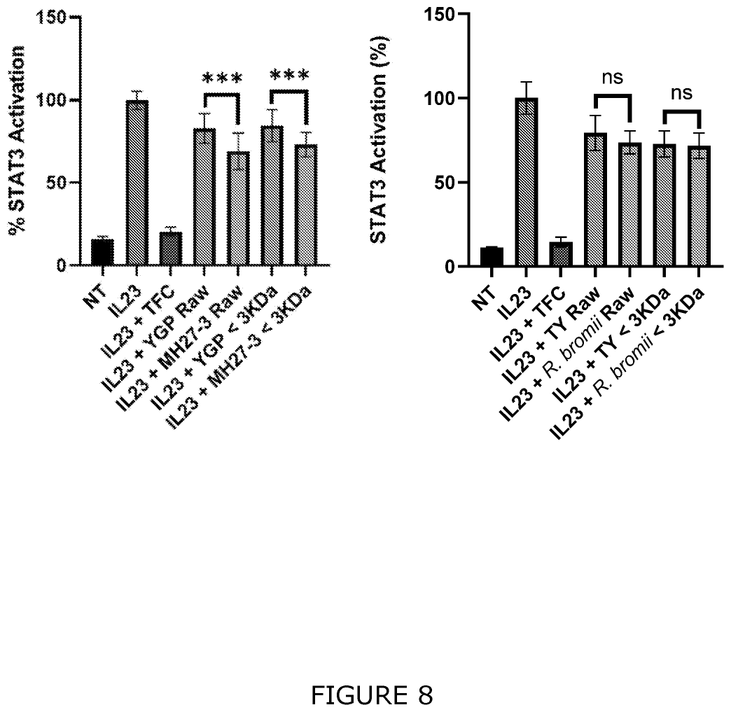

As expected, IL-23 mediated activation of STAT3 was fully prevented by tofacitinib (FIG. 8). I. colisanans MH27 cell-free culture supernatant and <3 kDa filtered culture supernatant significantly suppressed SEAP reporter activity, relative to the TY medium controls. In contrast, neither raw nor <3 kDa filtered supernatants prepared from Ruminococcus bromii MCB950 suppressed SEAP reporter activity, relative to the MCM medium controls (FIG. 8, right-hand graph).

These data demonstrate that the secretome of I. colisanans MH27, but not R. bromii MCB950, can suppress the IL-23/STAT3 signalling axis in vitro. Based on fractionation of the culture supernatant, the suppressive effects on STAT3 activation are likely mediated by one or more secreted low molecular weight bioactive(s).

I. colisanans Ameliorates IFNγ-Driven Reduction in Gut Barrier Integrity.

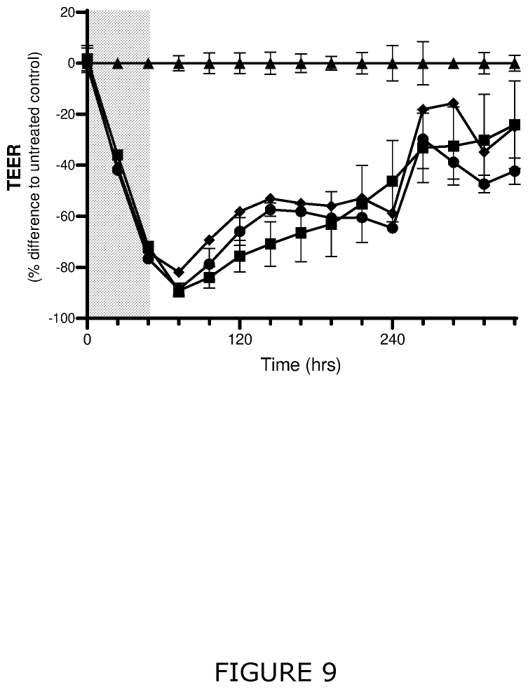

The ability of I. colisanans MH27 culture supernatant to modulate barrier function was examined using T84 cells. A basolateral treatment with IFNγ was used to disrupt barrier function. Following addition of IFNγ to T84 cells for 48 hours, there was a large drop in resistance indicating an increase in barrier permeability. Treatment with <3 kDa fractionated culture supernatant from I. colisanans MH27 ameliorated the reduction in TEER relative to the YG/V medium control most notably after 168 hours (FIG. 9, p<0.05 at 168, 192, 216, 240, 288, 312 and 336 hours as assessed by two-way ANOVA test).

I. colisanans ameliorates IL-6-driven reduction in gut barrier integrity.

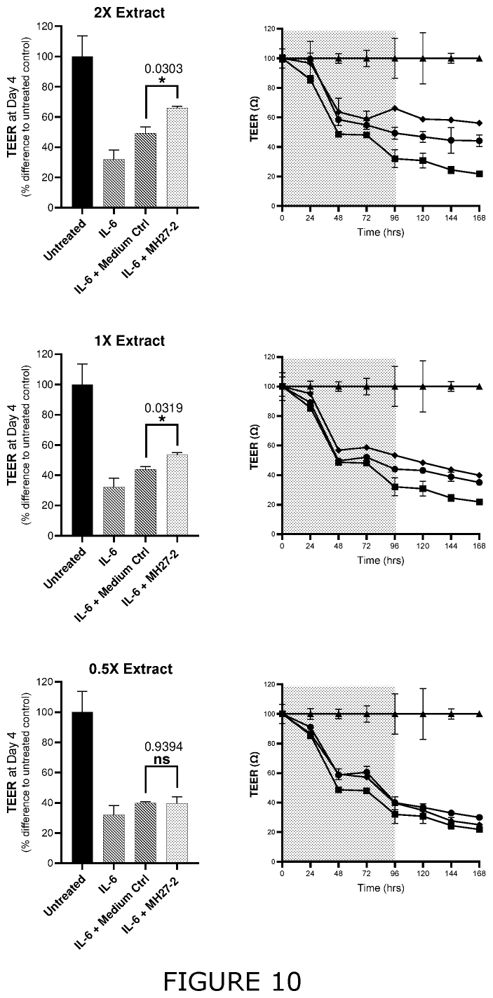

The ability of I. colisanans MH27-2 to modulate barrier function was examined by monitoring the TEER of T84 cells during and after inducing barrier integrity loss by the known barrier disruptor IL-6. The T84 cells were treated with IL-6 for 96 hours and, as expected, a significant drop in TEER could be observed indicating an increase in barrier permeability. Concurrent treatment with I. colisanans MH27-2 culture supernatant extract ameliorated the IL-6 mediated reduction in TEER in a dose dependent manner (FIG. 10).

I. colisanans Ameliorates IFNγ-Mediated Reduction of ZO1 Expression.

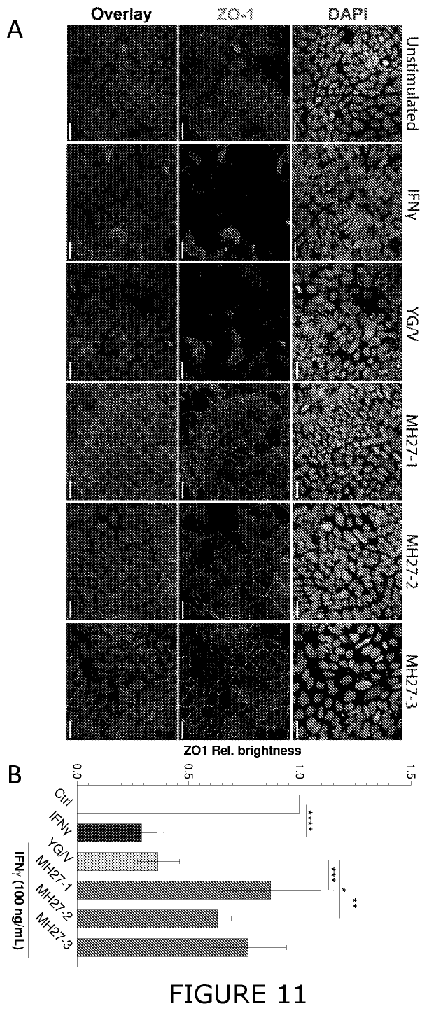

Tight junctions (TJs) form a continuous barrier between epithelial cells and act to control paracellular permeability. ZO1 plays a key role in tight junction assembly and we therefore assessed the ability of I. colisanans MH27 supernatant extract to mitigate IFNγ-mediated loss of ZO1 expression in T84 cells. Stimulation with IFNγ caused a marked reduction in ZO1 expression (FIG. 11). Pre-treatment with YG/V extract did not significantly affect the reduction in ZO1 expression (FIG. 11). In contrast, pre-treatment with I. colisanans MH27-1, MH27-2, and MH27-3 supernatant extract amelioratered the IFNγ induced reduction in ZO1 expression (FIG. 11).

I. colisanans Produces Metabolites that Modulate Gut Barrier Integrity.

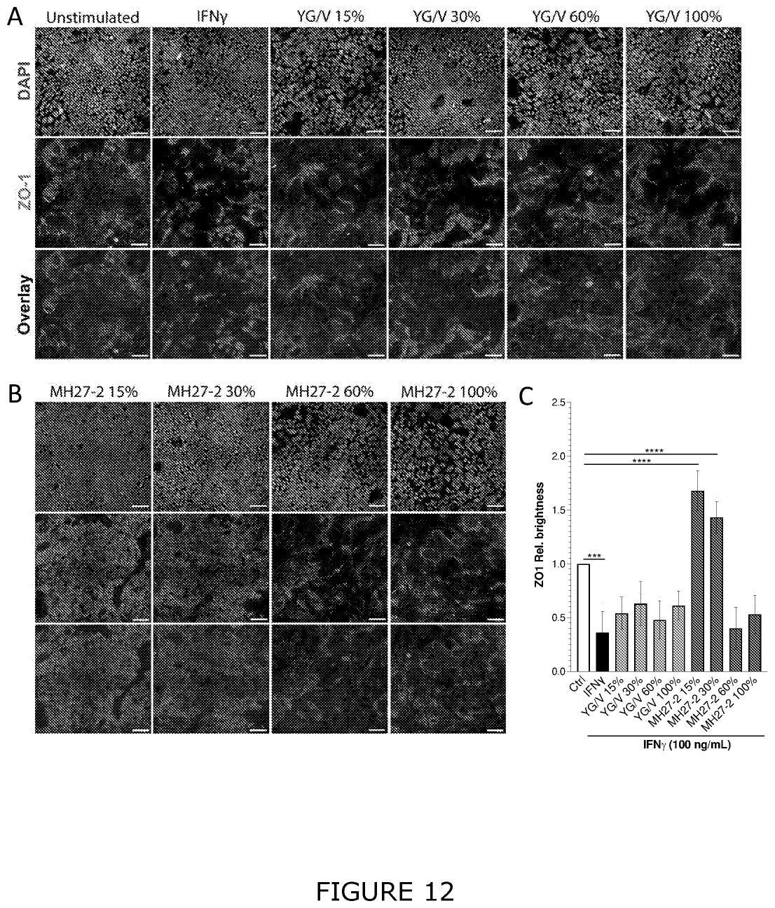

Based on the ability of I. colisanans supernatant extract to modulate ZO1 expression, we next examined the ability of Strata X-100 derived fractions of I. colisanans MH27-2 culture supernatant to modulate ZO1 expression. By this approach, we determined that the ability to modulate ZO1 expression could be fractionated and was localised to the 15- and 30% fractions (FIG. 12A-C).

### Metabolites Produced by I. colisanans MH27 Ameliorates IFNγ-Mediated Reduction of ZO1 Expression

Based on these data, the present inventors hypothesised that I. colisanans produces metabolites that modulate inflammatory responses and barrier integrity in gut epithelial cells. Thirty metabolites (classified as Level 1 or 2a) were identified in the cell free culture supernatant that were 1.5-fold increase relative to the YG/V medium control (Table 4). These included metabolites previously reported to modulate inflammation, immune cell infiltration, oxidative stress and barrier function (e.g., ornithine, indole-3-lactic acid, allopurinol, propionylcarnitine, pyrogallol, 3-(2-Hydroxyethyl)indole, and N-acetyl-cysteine, amongst others). A further 29 and 48 metabolites in the 15 and 30% fractions respectively that were ≥1.5-fold increased relative to the YG/V medium control (Table 4). These included metabolites previously shown to modulate inflammation and barrier integrity (e.g., tryptophol, indole-3-lactic acid, indole-3 propionic acid, cyclo(-Phe-Pro)). The immunomodulatory metabolite indole-3-acrylic acid was also identified in the 60% fraction. Note that some identified metabolites were not present in the control samples, and as such, a fold-change could not be calculated.

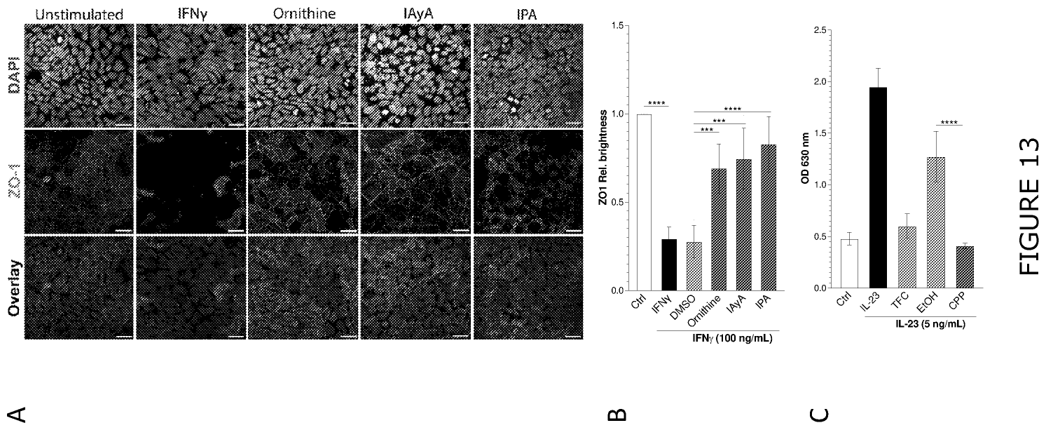

Having identified candidate gut barrier modulating metabolites, the present inventors next assessed the ability of select metabolites to modulate IFNγ-mediated loss of ZO1 expression in T84 cells. For this, T84 cells were pre-treated with ornithine, indole-3-acrylic acid (IAyA), or indole-3-proprionic acid (IPA) for 18 hours prior to stimulation with IFNγ for 48 hours. As previously observed, stimulation with IFNγ caused a marked reduction in ZO1 expression (FIG. 13A, B). Pre-treatment with ornithine, IAyA and IPA all significantly ameliorated the IFNγ-induced reduction in ZO1 expression (FIG. 13A, B). Separately, we also determined that cyclo(-Phe-Pro) suppressed IL-23 mediated activation of STAT3 (FIG. 13C).

TABLE 4
METABOLITES IDENTIFIED IN SUPERNATANT
(SN), 15%, AND 30% FRACTIONS

| | | Fold |
|---|---|---|
| Compound Name | Sample | change |
| Ornithine | SN | 3350.085 |
| N-Acetyl-glutamic acid | SN | 396.0299 |
| NAD+ | SN | 44.60856 |
| Succinic acid | SN | 42.68632 |
| Indole-3-lactic acid | SN | 27.72202 |
| Glutamine | SN | 16.17975 |
| Acetylhistidine | SN | 13.76723 |
| Creatinine | SN | 13.56073 |
| cis-Aconitic acid | SN | 7.736506 |
| 4-Hydroxy-2,5-dimethyl-3(2H)-furanone | SN | 7.589326 |
| 3-(3-Hydroxyphenyl)propionic acid/3-Phenyllactic acid/ | SN | 5.795158 |
| Acetovanillone/Ethyl vanillin | | |
| Homocysteine | SN | 5.53619 |
| N-Iso valerylglycine | SN | 3.820155 |
| Allopurinol | SN | 3.812495 |
| Propionylcarnitine | SN | 3.552227 |
| Crotonic acid | SN | 3.302462 |
| Pyrogallol | SN | 3.283553 |
| Inosine | SN | 3.255816 |
| N-Acetyl-tyrosine | SN | 2.713173 |
| 3-(2-Hydroxyethyl)indole | SN | 2.495905 |
| Riboflavin | SN | 2.41997 |
| N-Acetyl-phenylalanine | SN | 2.389627 |
| Dulcitol/Mannitol/Sorbitol | SN | 2.118445 |
| N-Acetyl-cysteine | SN | 1.924865 |
| N-Acetyl-methionine | SN | 1.923017 |
| Thymidine | SN | 1.847273 |
| N-Acetyl-lysine | SN | 1.771331 |
| N-Acetyl-tryptophan | SN | 1.760654 |
| Thymine | SN | 1.646492 |
| N-Acetyl-arginine | SN | 1.625908 |
| N-Caffeoylputrescine | 15% | — |
| 4-(2-oxo-3-hydrobenzoxazol-3-yl)-7,8- | 15% | 18.2 |
| dioxabicyclo[3.2.1]octan-2-one/maclurin | | |
| Chrysophanic acid | 15% | 12.8 |
| Cyclo-[L-(4-hydroxy-Pro)-L-leu]/Pyrrolo[1,2-a]pyrazine-1,4- | 15% | 10.7 |
| dione, hexahydro-3-(1-methylethyl) | | |
| Indole-3-lactic acid | 15% | 6.5 |
| GABA-C6:0 | 15% | 5 |
| Riboflavin | 15% | 4 |
| Tridecanedioic acid/Brassylic acid | 15% | 3.7 |
| 4-Hydroxymellein | 15% | 3.5 |
| N-Acetyl-L-tryptophan | 15% | 3.3 |
| Phe-Pro | 15% | 3 |
| Pterosin G | 15% | 2.8 |
| Gly-Phe | 15% | 2.7 |
| Tyramine | 15% | 2.6 |
| Cyclo(L-Val-L-Pro) | 15% | 2.5 |
| Tyr-Pro-Phe | 15% | 2.5 |
| L-Alanyl-L-norleucine/Cyclo-(Ala-Leu)/Cyclo-(Ala-4-hydroxy- | 15% | 2.2 |
| Pro) | | |
| Chelerythrine | 15% | 2.2 |
| L-Prolyl-L-isoleucine | 15% | 1.9 |
| Decanedioic acid/4-oxodecanedioate | 15% | 1.9 |
| Val-Ile | 15% | 1.8 |
| Phe-C4:0 | 15% | 1.8 |
| Diphenamid | 15% | 1.7 |
| Phenylalanine | 15% | 1.6 |
| N-Aceyl-L-tyrosine | 15% | 1.6 |
| Traumatic Acid/Butanedioic acid, 2-(4,4-dimethyl-2- | 15% | 1.6 |
| methylenepentyl) | | |
| 1-Hydroxyphenazine | 15% | 1.5 |
| 3-oxo-4-hydroquinoxaline-2-carboxylic acid/2,4- | 15% | 1.5 |
| Dichlorobenzoate | | |
| N-Acetyl-L-phenylalanine | 15% | 1.5 |
| Indole-3-propionic acid | 30% | — |
| Bisphenol A | 30% | — |
| 2-(2-acetyl-3,5-dihydroxyphenyl)acetate | 30% | — |
| Lidocaine N-Oxide | 30% | — |
| 3-(3,4,5-Trimethoxyphenyl)propanoic acid | 30% | — |
| Butanenitrile, 2-(beta-D-glucopyranosyloxy)-2-methyl-/ | 30% | — |
| Prunetin/Lotaustralin | | |
| PC(0:0/16:0) | 30% | — |
| Tryptophol | 30% | — |
| (3S,6R,9S,12R,15S,18R)-4,10,16-Trimethyl-3,6,9,12,15,18- | 30% | 130.5 |
| hexa(propan-2-yl)-1,7,13-trioxa-4,10,16-triazacyclooctadecane- | | |
| 2,5,8,11,14,17-hexone | | |
| 3-Phenyl-2-pyrrolylpropanoic acid | 30% | 102.4 |
| 2-[[2-(Diethylamino)acetyl]amino]-3-methylbenzoic acid/Ile- | 30% | 82 |
| Met | | |
| N-[1-carbamoyl-2-(4-hydroxyphenyl)ethyl]acetamide/Acetyl- | 30% | 43.7 |
| tyrosine | | |
| Gly-Ile/Leu-Gly/GABA-C5:0 | 30% | 41.2 |
| 2-Aminoanthraquinone | 30% | 31 |
| Methyl jasmonate | 30% | 22.6 |
| 2-Hydroxyoctanoic acid | 30% | 20.5 |
| (2-(4,4-Dimethyl-4,5-dihydrooxazol-2-yl)-5- | 30% | 16.6 |
| fluorophenyl)(phenyl)methanol | | |
| Etilefrine/N-Phenyldiethanolamine | 30% | 15.8 |
| Plumericin/Isoplumericin | 30% | 12.4 |
| Phe-C7:0/PyroGlu-Phe | 30% | 11.8 |
| Leu-C8:0/GABA-C10:0 | 30% | 10.8 |
| Isopentenyladenine | 30% | 10.2 |
| Isopentenyladenine | 30% | 7.9 |
| Curcumol/Palmitelaidic acid | 30% | 7.5 |
| 6-amino-1-phenyl-1,3-dihydropyrimidine-2,4-dione | 30% | 7 |
| 3-Hydroxycapric acid/10-Hydroxydecanoic acid | 30% | 6.4 |
| (1r,5R,7S)-5,7-Diethyl-2-(1-ethyl-2-methyl-1H-indol-3-yl)-1,3- | 30% | 5.8 |
| diazaadamantan-6-one | | |
| L-Kynurenine/Phenylalanine/2-Amino-1-phenylethanol/Indoline | 30% | 5.7 |
| 6,9-Dimethyl-3,4,5,3a,9b-pentahydrocyclopenta[1,2- | 30% | 5 |
| c]quinoline-4-carboxylic acid | | |
| 4-Methoxycinnamic acid/Mellein/2-Propenoic acid, 3-(4- | 30% | 4.5 |
| methoxyphenyl)-/3-Methoxycinnamic acid | | |
| (10E,15E)-9,12,13-Trihydroxyoctadeca-10,15-dienoic acid | 30% | 4.5 |
| Carveol | 30% | 4.1 |
| Zalcitabine | 30% | 4.1 |
| 10-Hydroxydecanoate/Citronellic acid | 30% | 3.9 |
| (3S,6R,6aS,7S)-6,6a,7,8-Tetrahydroxy-3-methyl- | 30% | 3.9 |
| 3,4,5,6,7,12a-hexahydro-2H-benzo[a]anthracene-1,12- | | |
| dione/Nifenazone/methyl 2-ethyl-4-[(3R,4R,5S)-5-hydroxy-4,5- | | |
| dimethyl-2-oxooxolan-3-yl]-2-methyl-3-oxobutanoate | | |
| Linalool/Geraniol | 30% | 3.6 |
| Traumatic Acid | 30% | 3.1 |
| FA 9:1 + 1O | 30% | 3 |
| Milrinone | 30% | 3 |
| Lenacil | 30% | 2.9 |
| Methylethyl (2E)-3-(3,5-dimethoxyphenyl)-2-cyanoprop-2- | 30% | 2.8 |
| enoate | | |
| Pyridoxine | 30% | 2.7 |
| 2,4,5-trimethoxybenzoic acid/Mescaline | 30% | 2.7 |
| Phe-Pro/cyclo(-Phe-Pro) | 30% | 2.4 |
| 3-Hydroxy-beta-lapachone | 30% | 2 |
| Cyclo-(Asn-Leu) | 30% | 1.9 |
| (10E,15E)-9,12,13-Trihydroxyoctadeca-10,15-dienoic | 30% | 1.7 |
| acid/14(Z)-Eicosenoic acid/FA 18:4 + 1O | | |
| 9-OxoOTrE/2H-Pyran-2-one, tetrahydro-4-hydroxy-6-[2- | 30% | 1.6 |
| (1,2,4a,5,6,7,8,8a-octahydro-2-methyl-1-naphthalenyl)ethyl]- | | |
| 2-Methoxycinnamaldehyde | 30% | 1.5 |

I. colisanans Exhibits Anti-Inflammatory IL-10/IL-12 Cytokine Ratio

Previous studies have shown that the ability of bacteria to induce secretion of IL-10 and IL-12 from PBMCs in vitro correlates with their in vivo immunomodulatory capacity (Foligne et al., 2007; and Sokol et al., 2008). In particular, the IL-10:IL-12 ratio is widely used to differentiate between strains showing an anti-inflammatory (high IL-10:IL-12 ratio) or proinflammatory (low IL-10:IL-12 ratio) with a mean IL-10:IL-12 ratio >5 correlating with protection against TNBS-induced experimental colitis. The ability of I. colisanans to induce cytokine production from PBMCs was therefore assessed.

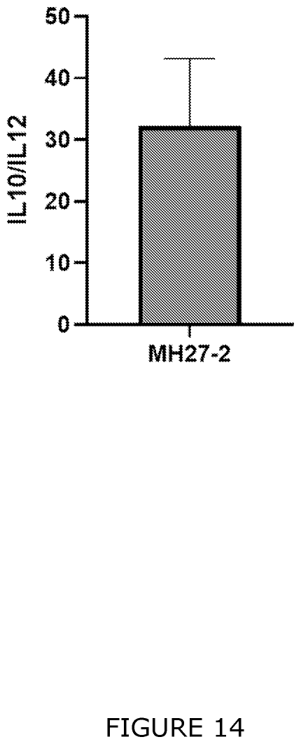

Treatment with I. colisanans resulted in an IL-10:IL-12 ratio of 30 (FIG. 14) which further demonstrates the strong anti-inflammatory activity observed in the above data.

I. colisanans Exhibits NF-κB Suppressive Activity

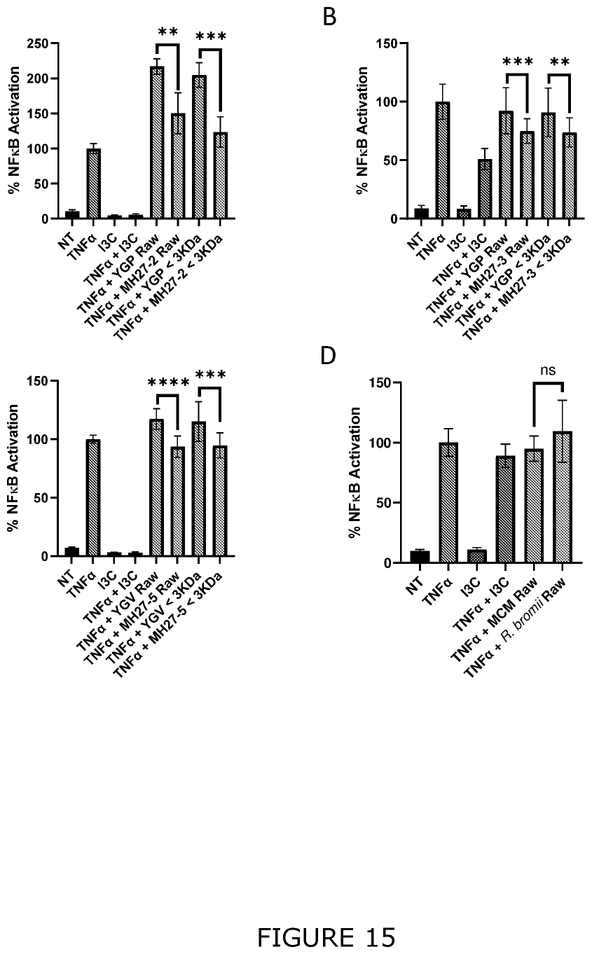

The NF-κB signalling axis plays a key role in the pathogenesis of inflammatory bowel disease (IBD) and it is a recognised and validated therapeutic target. We show that I. colisanans MH27-2, MH27-3 and MH27-5 cell free culture supernatant and <3 kDa filtered culture supernatant significantly suppressed luciferase reporter activity, relative to the medium controls. In contrast, raw supernatant prepared from R. bromii MCB950 suppressed reporter activity, relative to the medium controls (FIG. 15A,D).

### Materials & Methods

### Bacterial Strains, Culture Conditions and Analyses.

Stool samples were collected from healthy human adults with no history of gastrointestinal disorders and mixed with an equal weight per volume of sterile oxygen free glycerol solution (McSweeny, 2005). Donors had not consumed antibiotics in the 3 months prior to collection of the faecal samples. All samples were collected following informed consent and the ethical guidelines approved by Bellberry Limited (HREC2018-05-400-A-6). I. colisanans and Faecalibacterium prausnitzii were routinely processed in a Coy vinyl anaerobic chamber with an oxygen free atmosphere (85% N₂:10% CO₂:5% H₂) atmosphere. I. colisanans was routinely cultured in TY or YG/V medium (Table 1) while F. prausnitzii was cultured in TY medium (Table 1). All isolates were stocked by mixing 3 mL of actively growing culture with an equal volume of glycerol solution and storing at −80° C.

Isolation of I. colisanans.

An isolate of I. colisanans was produced by inoculating a donor faecal sample with I. colisanans present at a relative abundance of 0.37% into custom medium (Trehalose 1 g/L, Alanine 400 mg/L, Tryptophan 80 mg/L, Methionine 200 mg/L, Phenylalanine 200 mg/L, Tyrosine 200 mg/L, Butyric acid 400 μL/L, Salts 2 75 ml/L, Salts 3 75 ml/L, Sodium bicarbonate 8 g/L, Resazurin 1 mL/L, Cysteine hydrochloride 1 mL/L, Vitamin solution 1 ml/L) and then serially diluting to extinction. The dilution-to-extinction culture series was sequenced and a low diversity enrichment culture with I. colisanans at a relative abundance of 77% was identified. This enrichment culture was then used to establish a further dilution-to-extinction culture series and a low diversity enrichment with I. colisanans at over 79% was identified. Colonies were recovered on PYG agar and subsequently picked into PYG broth. From this, a bi-culture comprised of I. colisanans and Ruthenibacterium lactatiformans was identified. To produce an axenic culture of I. colisanans, the bi-culture was inoculated into YCFAmod broth and the enrichment culture was then streaked onto YCFAmod agar. Two distinct colony types were observed after several days, and these were purified. By this approach, a Gram-variable staining coccoid isolate was identified and termed I. colisanans MH27-1 following whole genome sequencing.

The isolate of I. colisanans MH27-2 was produced by inoculating a donor faecal sample with I. colisanans present at a relative abundance of 0.38% into custom medium (Maltose 1 g/L, Alanine 400 mg/L, Tryptophan 80 mg/L, Methionine 200 mg/L, Phenylalanine 200 mg/L, Tyrosine 200 mg/L, Histidine 125 mg/L, Salts 2 75 ml/L, Salts 3 75 ml/L, Sodium bicarbonate 8 g/L, Resazurin 1 mL/L, Cysteine hydrochloride 1 mL/L, Vitamin solution 1 ml/L) and then serially diluting to extinction. The dilution-to-extinction culture series was sequenced and an enrichment with I. colisanans at a relative abundance of 72.6% was identified. The enrichment was streaked on a PYG plate and an isolate with a coccoid cell morphology was identified. Following purification, a Gram-variable staining coccoid isolate was identified and termed I. colisanans MH27-2 following whole genome sequencing.

The isolate of I. colisanans MH27-3 was produced by inoculating a donor faecal sample with I. colisanans present at a relative abundance of 0.38% into custom medium (Maltose 1 g/L, Alanine 400 mg/L, Tryptophan 80 mg/L, Methionine 200 mg/L, Phenylalanine 200 mg/L, Tyrosine 200 mg/L, Histidine 125 mg/L, Salts 2 75 ml/L, Salts 3 75 ml/L, Sodium bicarbonate 8 g/L, Resazurin 1 mL/L, Cysteine hydrochloride 1 mL/L, Vitamin solution 1 ml/L) and then serially diluting to extinction. The dilution-to-extinction culture series was sequenced and an enrichment with I. colisanans at a relative abundance of 14.5% was identified. This enrichment was used to inoculate a second dilution-to-extinction experiment in the same GDI medium resulting in a new 28% enrichment of I. colisanans and other bacterial species at <27% relative abundance. The enrichment was plated on YG/V and a single colony comprised of Gram-variable staining coccoid cells was identified. The colony was picked and streaked twice more on YG/V agar. The axenic isolate was termed I. colisanans MH27-3 following whole genome sequencing.

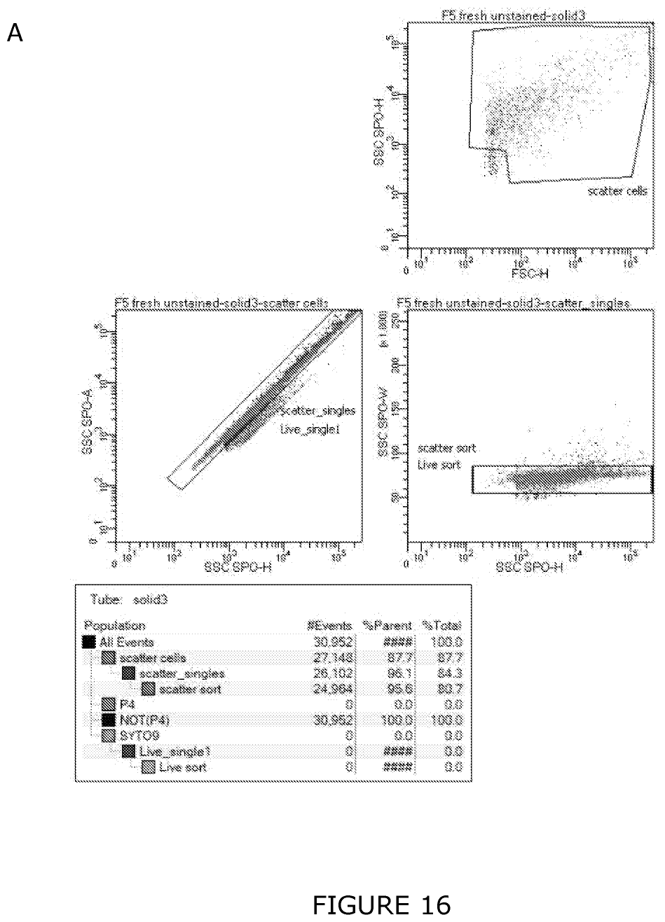

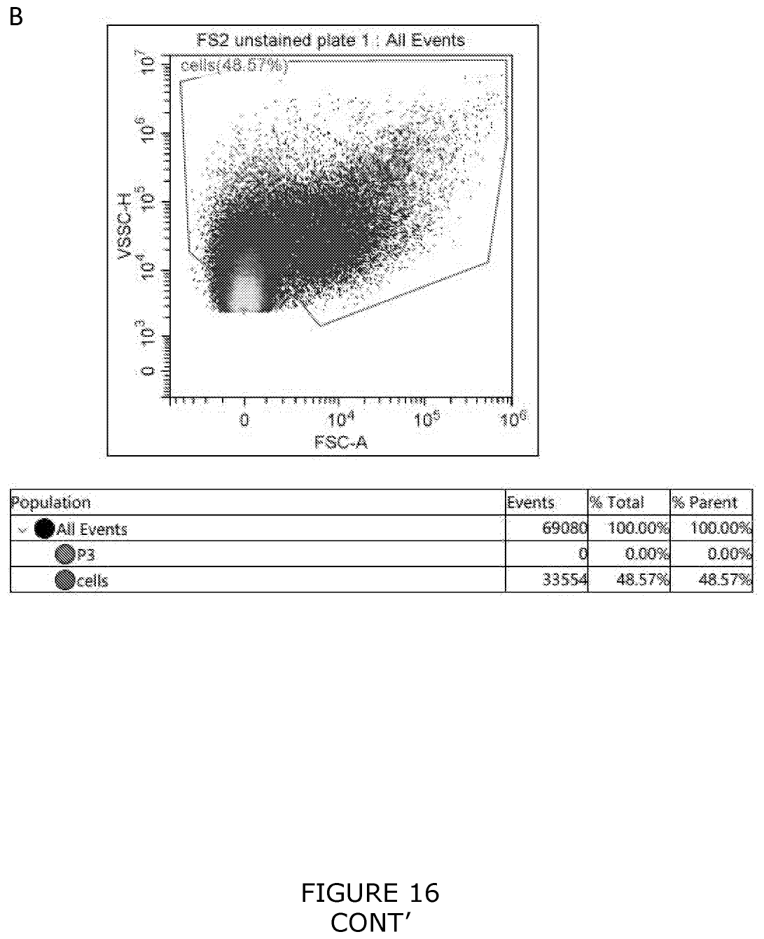

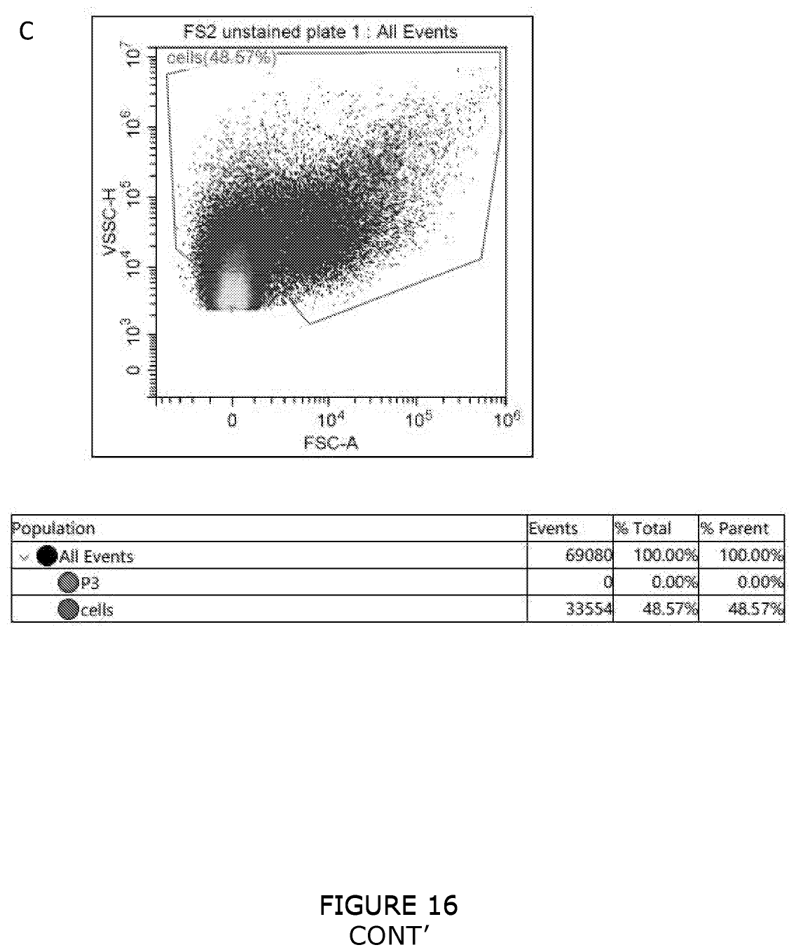

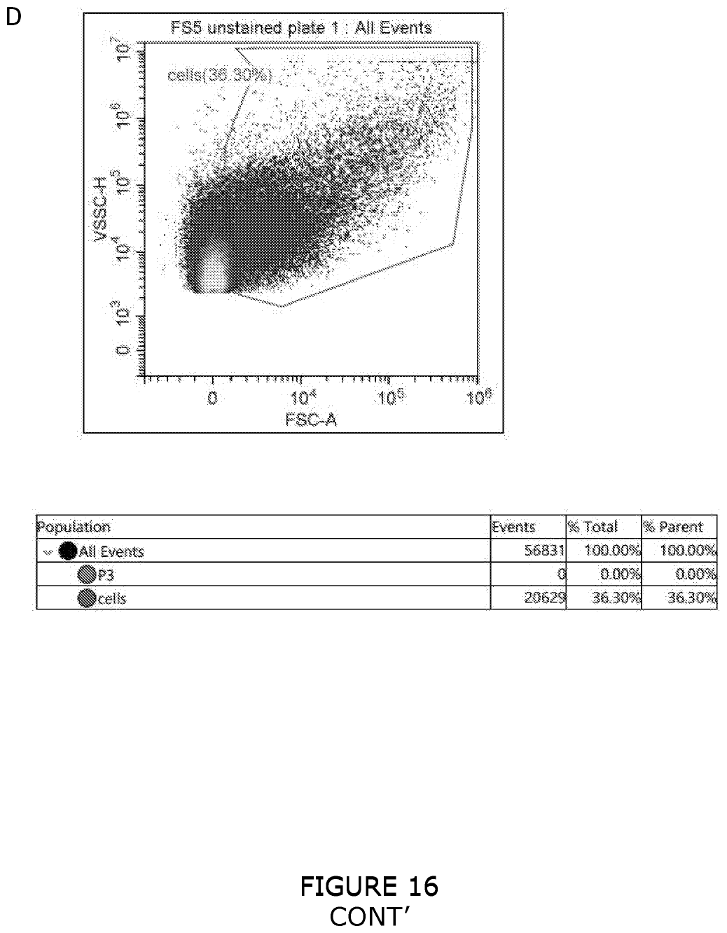

I. colisanans MH27-4, MH27-5 and MH27-6 were produced from donor faecal samples using a microbial based single cell sorting approach. For I. colisanans MH27-4, approximately 1 g of a faecal sample with I. colisanans at 0.3% relative abundance was mixed with 1 mL of anaerobic buffered diluent solution and vortexed for 10 seconds to dissociate bacteria from faecal debris. For I. colisanans MH27-5, approximately 1 g of a faecal sample with I. colisanans at 0.67% relative abundance was mixed with 1 mL of anaerobic buffered diluent solution and vortexed for 10 seconds to dissociate bacteria from faecal debris. For I. colisanans MH27-6, approximately 1 g of a faecal sample with I. colisanans at 2.5% relative abundance was mixed with 1 mL of anaerobic buffered diluent and vortexed for 10 seconds to dissociate bacteria from faecal debris. Ruminococcus bromii MCB950 was similarly produced from donor faecal samples using a microbial based single cell sorting approach and used for comparative analyses. For R. bromii MCB950, approximately 1 g of a faecal sample with R. bromii at 5.5% relative abundance was mixed with 1 mL of anaerobic buffered diluent solution and vortexed for 10 seconds to dissociate bacteria from faecal debris. Bacterial cells were sorted using a BD FACSAria™ Fusion Flow Cytometer with a 100 μm nozzle and small particle detector according to the manufacturer's instruction. The system was pressurised at 20 psi and events were triggered on scatter with thresholds set using 0.2 μm filtered PBS which allowed a few events from the noise floor. Bacterial cells were also sorted using the CytoFLEX SRT Benchtop Cell Sorter inside a COY anaerobic chamber. The instrument was set up and quality controlled according to manufacturer's instructions. Dry ice was included inside the anaerobic chamber to keep the instrument within a set temperature range. The sheath of the Fusion and SRT was sparged with nitrogen gas for one hour prior to sorting. To sort bacterial cells, an aliquot was filtered through a 40 μm filter and a 10-3 dilution of the filtrate was subsequently prepared using a 0.2 μm filtered anaerobic buffered diluent solution (i.e., 38 ml/L each of salt solutions 2 & 3 (McSweeney et al., 2005)). The diluted filtrate was run to determine the dilution resulting in 500-1200 events per second. Targets were gated on scatter and doublets were excluded (FIG. 16). Single cells were deposited onto TY agar plates and incubated at 37° C. for up to 3 weeks. Isolates were identified by whole genome sequencing following growth in TY broth. By this approach, isolates affiliated with I. colisanans and R. bromii were tentatively identified. The I. colisanans isolates were purified by repeated streaking of single colonies on YG/V agar and the R. bromii were purified by repeated streaking of single colonies on TY agar. The final purified isolates were glycerol stocked and submitted for whole genome sequencing and by this approach isolates termed I. colisanans MH27-4 and MH27-5 were produced.

For I. colisanans MH27-6, following growth of the cell sorted isolates in TY broth, an enrichment containing I. colisanans MH27-6 at a relative abundance of 10.6% was identified. From a glycerol stock of this enrichment, 200 μl was used to inoculate YG/V medium and I. colisanans was enriched to 71.5% relative abundance. I. colisanans colonies were produced and subsequently purified by streaking on YG/V agar. The final purified isolate was glycerol stocked and submitted for whole genome sequencing and by this approach an isolate termed I. colisanans MH27-6 was produced.

### Metabolic Reconstruction.

Protein coding sequences were predicted and annotated using the annotate function in enrichM (0.6.2). Briefly, enrichM identifies protein coding sequences using prodigal (version) in −p meta mode. The amino acid sequences are then searched against the UniRef100 database (version) using DIAMOND (version), and E.C., TCDB and eggnog classifications are inherited from the id mapping file distributed with UniRef. Hmmer hmmsearches (version 3.1b2) against Pfam (release 33.0), TIGRFAM (release 15.0) and dbCAN2 (downloaded September 2019) were used to annotate functional domains, key metabolic markers and carbohydrate activate (CAZy) enzymes, respectively. Metabolic pathways were identified using the classify function in enrichM, which assesses annotations and their genomic position against manually defined metabolic pathway definitions. A pathway is considered present in a genome if it encoded >80% of the required proteins and passes all required synteny checks. These automatically predicted pathways were then manually assessed. In addition, gutSMASH (version 1.0.0) was applied to identify common functions mediated by gut microbiomes.

### Phyloaenetic Trees.

Clade 23560 in the species representative tree of GTDB r89 was visualised using the R libraries phytools (v0.7.70), ape (v5.4) and ggtree (v2.2.4). A separate genome tree was constructed from high quality genomes (≥90%, contamination and ≤55% contamination from checkM analysis) within the Intestinococcus genus (NCBI r89) and the MH27 isolate. For each genome, a set of 122 bacteria-specific conserved marker genes were extracted from each genome using gtdbtk identify. These genes were the aligned to profile HMMs and concatenated to a single alignment with gtdbtk align, and a Maximum likelihood phylogenetic tree was constructed from the alignments using FastTree (v2.1.10) with gtdbtk infer. Non-parametric bootstrap values were inferred using GenomeTreetk (v0.1.6) from 1000 repetitions.

### Preparation of Bacterial Strains for Animal Experimentation.

I. colisanans strains were grown to early stationary phase in PYG or YG/V. F. prausnitzii was grown in TY medium. The cell density of the individual cultures was calculated using a Helber Counting Chamber. To prepare the bacterial gavage solutions, individual cultures were centrifuged under a layer of sterile heavy mineral oil at 5,000 g for 10 minutes and the cell-free supernatant was then discarded. The cell pellets were washed in 1.5 ml of sterile anaerobic buffered diluent (38 ml/L each of salt solutions 2 & 3 (McSweeney, 2005), 1 ml/L of 0.1% (w/v) resazurin solution, 1 g/L L-cysteine) and then centrifuged again. Finally, the washed cell pellet was resuspended in half strength glycerol solution (15% v/v glycerol solution in anoxic buffered diluent) to a final concentration of 1×10⁹ cells/ml, aliquoted and frozen at −80° C. until required. The viability of the cell preparations was confirmed by thawing a single aliquot and streaking on an agar plate. The identity and purity of the individual strain preparations was confirmed by whole genome sequencing.

### Acute Model of DSS Induced Colitis.

For the prophylactic model, six-week-old C57BL/6 female mice purchased from Animal Resources Centres (Western Australia, Australia) were randomised and then co-housed for 7 days prior to experimentation. To induce disease, mice were treated with 3.5% DSS ad libiteum in the drinking water for 6 days. Naïve age matched control mice were processed and received DSS free drinking water. All treatments started 1 day prior to provision of DSS and all mice were sacrificed 2 days after the final DSS treatment. For the treatments, mice were anesthetised with isofluorane and orally gavaged with 200 μL of bacterial preparations or vehicle control. Prednisone (2 mg/kg) was administered following anesthetisation by intraperitoneal injection. Body weights and stool consistency were recorded daily. Stool samples were collected daily. Following sacrifice, the colon, liver and spleen were collected for analysis. Blood was collected by cardiac puncture.

For the therapeutic model, groups of 10 or 20 female C57BL/6 mice were used. All animals, except those in the healthy group, were given 2.5% dextran sodium sulfate (DSS) in the drinking water for 6 days total (Day 0 to Day 5). DSS was then withdrawn and replaced with normal drinking water for a further 5 days (Day 6 to Day 10). For the treatments, mice were given once daily oral gavage of 200 μL of I. colisanans MH27-2 at a dose of 2×10\hat{ }8 viable cells per day, or vehicle consisting of sterile glycerol and phosphate buffered salt solution. The positive control drug prednisone was similarly administered via oral gavage at a dose of 2 mg/kg per day. Treatments started on Day 4 and were given for 7 consecutive days until Day 10. Animals were sacrificed on Day 10 approximately 4 hours after the final treatment. Colon was harvested, rinsed, weighed, photographed, cut longitudinally. Colon tissue was fixed in 4% formalin and kept in 70% ethanol for histopathology using the Swiss-roll method.

### Endoscopic and Histological Scoring.

Animals were examined with a small animal gastrointestinal endoscope (Karl Storz Endoskope, Tuttlingen, Germany) on days −1, 2 and 6 to assess the extent of colon mucosal inflammation (Marks, 2015; and Liu, 2019). Briefly, mice were anesthetised with isoflurane and a colonoscope was inserted through the rectum. Images captured by high-definition videos were examined in a blinded manner to assess the presence and extent of disease pathologies (Table 5). Histological scoring was performed essentially as described by Marks et al. (Marks, 2015). Briefly, samples were fixed in 4% formalin, paraffin embedded and sectioned. Tissue sections were hematoxylin and eosin stained to assess disease pathology and with Alcian blue to assess mucin production. Slides were imaged using the Aperio digital imaging system (Leica Biosystems, NuBloch, Germany). To grade colitis severity, the extent of inflammation and epithelial injury in the tissue sections were graded semi-quantitatively using an established scoring system (Table 6). The samples were then randomised and subsequently scored in a blinded by a trained gastrointestinal pathologist.

### SKG Model of Murine Ileitis.

Five-week-old SKG female mice (n=3 male; n=3 female) were randomised and then co-housed for 3 weeks prior to experimentation. To induce disease, mice were administered curdlan (1,3-β-glucan, 3 mg per mouse) intraperitoneal (i.p.). Naïve age matched control mice were similarly processed except that they were administered saline i.p. All treatments started prior to administration of curdlan, and all mice were sacrificed 7 days after the curdlan treatment. For the bacterial treatments, mice were orally gavaged with 200 μl of bacterial preparations or vehicle control starting 2 days prior to the curdlan administration. Anti-mouse IL-23 p19 monoclonal antibody (Thermo Fisher, 30 μg per mouse) was administered by i.p. injection one day prior to curdlan administration. Stool samples were collected daily. Body weights were measured at Day −2. 0 and 7. Following sacrifice, the colon, distal small intestine, mesenteric lymph nodes and spleen were collected for analysis. Blood was collected by cardiac puncture.

### Clinical and Histological Scoring.

Histological scoring was performed essentially as described by Benham et al. 2014. Briefly, samples were fixed in 4% formalin, paraffin embedded and sectioned. Tissue sections were hematoxylin and eosin stained to assess disease pathology and with Alcian blue for goblet cell assessment. Slides were imaged using an Olympus VS120 slide scanner (Olympus Corporation, Tokyo, Japan). To grade ileitis severity, the extent of inflammation and epithelial injury in the tissue sections were graded semi-quantitatively using an established scoring system (Table 7) (Benham et al., 2014). The samples were then randomised and subsequently scored in a blinded manner by an independent trained pathologist.

To characterise the cytokines present in serum, the mice were euthanised by CO₂ asphyxiation after which blood was collected by cardiac bleed and serum isolated by centrifugation. Serum samples were stored at −80° C. These serum samples were subsequently thawed and used in the LEGENDplex Mouse Inflammation Panel (13-plex) (BioLegend, Cat. 740446) to determine effects on cytokine production (IL-23, IL-1α, IFN-γ, TNF, MCP-1, IL-12p70, IL-1β, IL-10, IL-6, IL-27, IL-17A, IFN-R, GM-CSF). Serum samples were diluted 2-fold and then incubated with capture beads conjugated to an antibody against specific analyte. After washing, a biotinylated detection antibody mixture is added to create a “capture bead-analyte-detection antibody” sandwich. Streptavidin-phycoerythrin (SA-PE) is added to bind to the biotinylated detection antibodies. After a final wash, the mixtures are analyzed by flow cytometry where the beads are differentiated by size and internal fluorescence intensities. Cytokine concentration is determined by PE fluorescence and comparison to a standard curve of known cytokine concentrations using BioLegend's LEGENDplex data analysis software. The unique size and fluorescence characteristics of the beads allows for the simultaneous measurement of 13 cytokines.

### TNBS Model of Murine Colitis

Groups of 5 or 10 male BALB/c mice were used. Mice were fasted overnight (Day 1) before 2,4,6-Trinitrobenzene sulfonic acid solution (TNBS) challenge on Day 2. Distal colitis was induced by intracolonic instillation of TNBS (1 mg in 0.1 mL 50% ethanol) after which, animals were kept in a vertical position for 30 seconds to ensure that the solution remained in the colon. For the treatments, mice were orally gavaged with 200 μl of I. colisanans MH27-2 at 1×10\hat{ }9 cells/day or vehicle (sterile glycerol and phosphate salt solution) starting from Day 1 (i.e., 1 days before TNBS) to Day 5 for a total of 5 consecutive days (Days 1-5). The positive control Cyclosporin A was given at 75 mg/kg by oral gavage once daily from Day 1 (i.e., 1 days before TNBS) to Day 4 for a total of 4 consecutive days (Days 1-4). On the Day of TNBS challenge, vehicle, I. colisanans MH27-2 and Cyclosporine A were given at 2 hours before TNBS. Animals were sacrificed on Day 5; blood was collected from all animals by cardiac puncture. The colon tissue was also harvested and snap frozen with liquid nitrogen for cytokine measurement. On Day 5, the mice were euthanized by CO₂ asphyxiation. Each colon was removed, rinsed, and then cut from 4 cm from the anus. The tissue sections were fixed in 10% formalin and kept in 70% ethanol for histopathology.

### Macroscopic Scoring of TNBS Colitis

On Day 5, the mice were euthanized by CO₂ asphyxiation, colons weighed and their lengths measured. Furthermore, when the abdominal cavity was opened before removal of the colon, adhesions between the colon and other organs were noted as was the presence of colonic ulceration after removal and weighing of each colon. Macroscopic scoring was performed (Table 8), and photos of the intact colons taken.

### Histopathological Scoring of TNBS Colitis

Each colon was removed, rinsed, macroscopically scored, photographed, weighed and its length measured, and then cut to two parts from 4 cm from the anus; one part fixed in 10% formalin and kept in 70% ethanol for histopathology, another part snap frozen with liquid nitrogen for cytokine measurement (IL-6). For histological analysis (Colitis scoring; essentially as described by Dieleman L A et al., 1998), four-micrometre tissue sections were cut and stained with hematoxylin and eosin (H&E) under light microscope (LEICA DM2700 M, USA). Histological criteria included: abnormalities of mucosal architecture, extent of inflammation, erosion or ulceration, epithelial regeneration, and the percentage involvement by the disease process. The scoring was based on the findings of observers by examining two sections from each colon per animal. Total score for colitis (Total Colitis Index) were added, resulting in a combined histologic score range from 0 to 40 (Table 9). Cytokines were measured using a Millipore ELISA kit and a protocol adapted from the product information and manual. Score between 0-20 for 2× sections and add scores for a total range 0-40.

### Cell Migration Analysis.

The TRANSWELL migration assay was used to assess the migration of HCT116 cells during exposure to I. colisanans culture supernatant extracts. These bacterial extracts were prepared using an Amberlite XAD-7 resin essentially as previously described by Colosimo et al. (Colosimo, 2019). Briefly, a single colony was inoculated and grown until early stationary phase. This “seed culture” broth was used to inoculate 600 mL of TY broth and the culture was incubated until early stationary phase. Culture supernatants were prepared by centrifuging the culture at 4000 g for 30 minutes and then passing the cell free supernatant through a 3 kDa filter according to the manufacturer's instructions (Sartorius Vivaflow® 50 Ultrafiltration Unit 3 kDa MWCO PES). Activated Amberlite XAD-7 resin was added to 400 mL of 3 kDa filtered cell-free supernatant (10% w/v), and the slurry was gently shaken overnight at 4° C. The resin was collected, washed with 400 mL of deionized water and then mixed with 120 mL of 100% methanol. Following a 2 hour incubation with gentle shaking, the methanol elution was collected. A second elution in 120 mL of 100% methanol was performed as previously described and the two elutions were ultimately combined and dried under vacuum using a rotary evaporator. The extract was fully resuspended at in 100% DMSO (thereafter referred to as 1000×) and stored at −20° C.

Human HCT116 gut epithelial cells were maintained in McCoys 5a medium supplemented with 10% FBS and 1% Pen/Strep. To assess cell migration via the Transwell® assay, 3.5×10⁴ HCT116 cells were seeded in 100 μL 10% FBS culture medium in the top compartment of a 6.5 mm insert with TC-treated polycarbonate membrane in 24-well plates (8 μm pore size, Corning Costar). 600 μl 10% FBS culture medium was added to the lower compartment. The cells were allowed to settle for 24 hours. After a DPBS wash, 100 μL and 600 μL of 0.5% FBS medium was added to the top and lower compartment, respectively. Then, 0.5× concentrated extract from I. colisanans was added to the lower compartment. After 16 hours, the cells were washed with DPBS and the cells attached to the top of the membrane were carefully removed with a cotton tip. The migrated cells on the bottom of the membrane were then fixed in 70% ethanol for 10 minutes followed by staining in 0.25% crystal violet for 5 minutes. The Transwell® inserts were washed with water, dried and the membrane mounted with 50% glycerol in water on glass slides and imaged immediately. Transwell® experiments were performed in biological and technical triplicates and for each replicate two representative images of the membrane were taken at 10× magnification. The number of migrated cells was automatically counted using ImageJ and the average cell number displayed. The extent of cell migration was expressed as the average number of migrated cells in two microscopic fields per well from 3 biological and 3 technical replicates.

The IncuCyte® Live-Cell Imaging System (Essen BioScience) and transwell migration assay were used to assess the migration of HCT116 cells during exposure to sterile culture supernatant from I. colisanans. Human HCT116 gut epithelial cells were maintained in McCoys 5a medium supplemented with 10% FBS and 1% Pen/Strep. For the IncuCyte® scratch wound assay, 3.5×10⁴ HCT116 cells were plated on poly-L-ornithine-coated IncuCyte® ImageLock 96-well plates (Essen BioScience). After 24 hours, the IncuCyte® WoundMaker tool was used to induce a homogeneous scratch wound in the nearly confluent cell monolayer. The cells were washed twice with DPBS and treated with I. colisanas supernatant extracts (prepared using an Amberlite XAD-7 resin as previously in section “Preparation of bacterial supernatant and medium extract” below) at 0.3× in 200 μL 0.5% FBS McCoys 5a medium was added. Similarly prepared bacterial medium extract served as negative control. Immediately after adding the stimulants, the plate was transferred to the IncuCyte® system and cell migration was monitored by imaging each well every 2 hours over the course of 72 hours. Data analysis was performed using the integrated analysis software.

TABLE 5
SCORING OF GUT BARRIER DYSFUNCTION
BY COLONOSCOPY

| | |
|---|---|
| Feature | Scoring |

Thickening of the colon wall

| | |
|---|---|
| Transparent | 0 |
| Moderate | 1 |
| Marked | 2 |
| Intransparent | 3 |

Changes in vascular pattern

| | |
|---|---|
| Normal | 0 |
| Moderate | 1 |
| Marked | 2 |
| Bleeding | 3 |

Granularity of the mucosal surface

| | |
|---|---|
| None | 0 |
| Moderate | 1 |
| Marked | 2 |
| Extreme | 3 |

Stool consistency/mucus secretion

| | |
|---|---|
| Normal and solid | 0 |
| Still shaped, mild mucus | 1 |
| Unshaped, mucus | 2 |
| Spread | 3 |

Extent of involved area

| | |
|---|---|
| 0-5% (none) | 0 |
| 5-20% (patchy) | 1 |
| 20-50% (moderate) | 2 |
| >50% (predominant) | 3 |

TABLE 6
SCORING OF HISTOLOGICAL COLITIS
Inflammation score (Scored 0-4)

| | |
|---|---|
| 0 | No evidence of inflammation |
| 1 | Low level of inflammation with scattered |
| | infiltrating mononuclear cells (1-2 foci only) |
| 2 | Moderate inflammation with multiple foci |
| 3 | High level of inflammation with increased vascular |
| | density and marked wall thickening |
| 4 | Maximal severity of inflammation with transmural |
| | leukocyte infiltration and loss of goblet cells |

Injury score (Scored 0-3)

| | |
|---|---|
| 0 | No epithelial injury |
| 1 | Occasional epithelial lesion |
| 2 | 1-2 foci of ulceration |
| 3 | Extensive ulceration |

Colitis activity (Composite score (/17)
based on measures listed below)

| | |
|---|---|
| Hypervascu- | 0-3 based on severity |
| larisation | |
| Presence of | 0-3 based on severity |
| mononuclear cells | |
| Epithelial | 0-3 based on severity |
| hyperplasia | |
| Epithelial | 0-3 based on severity |
| injury | |
| Presence of | 0-3 based on severity |
| neutrophils | |
| Lymphoid | Scored 0-2, where 0 = 0, 1 = ≤2, and 2 = ≥2 |
| aggregates | |

TABLE 7
HISTOLOGICAL SCORING SYSTEM
FOR ILEUM OF SKG MICE

| Feature | Grade | Description |
|---|---|---|
| Inflam- | 0 | Absent |
| matory | 1 | Scattered PMNs or MNs in lamina propria (LP) |
| infiltrate | | involving <5 intercryptal spaces contiguously |
| | | involved in an area |
| | 2 | Increased PMNs or MNs in LP and/or submucosa |
| | | involving >5 intercryptal spaces contiguously |
| | | involved in an area and ± crypt abscesses |
| | | and areas of crypt/villous loss <5 crypt widths |
| | 3 | Extensive transmural inflammatory infiltrate and |
| | | areas of crypt/villous loss >5 crypt widths and/or |
| | | confluent areas of crypt abscesses and/or mucosal |
| | | erosion/ulceration |
| Granulomata | 0 | Absent |
| | 1 | Aggregation of cells not definitely a granuloma |
| | 2 | A definite granuloma formation present |
| | 3 | Multiple granulomas present |
| Villous | 0 | Absent |
| Distortion | 1 | Villus height increased or decreased by <⅓ of |
| | | normal |
| | 2 | Villus height increased or decreased by ⅓-⅔ of |
| | | normal |
| | 3 | Villus height increased or decreased by >⅔ of |
| | | normal |

| Cross sectional | |
|---|---|
| area | Score |
| <1% | 0.5 |
| 1-25% | 1 |
| 26-50% | 2 |
| 51-75% | 3 |
| >75% | 4 |

For total histology score add scores from sections 1, 2 and 3 and multiply by cross sectional area involved.

TABLE 8
SCORING OF MACROSCOPIC COLITIS
The criteria for macroscopic scoring of colonic
damage are described as follows: Score

| | | |
|---|---|---|
| Adhesions | None | 0 |
| | Minimal (colon could be | 1 |
| | separated easily from other tissue) | |
| | Involving several bowel loops | 2 |
| Strictures | None | 0 |
| | Mild | 2 |
| | Severe, proximal dilatation | 3 |
| Ulcers/ | No damage | 0 |
| Inflammation | Focal hyperemia; no ulcers | 1 |
| | 1 site of ulceration/inflammation <1 cm | 2 |
| | 2 sites of ulceration/inflammation <1 cm | 3 |
| | Major site(s) of ulceration/inflammation >1 cm | 4 |
| | If damage >2 cm increase score by 1 for | 5+ |
| | each additional cm of damage | |
| Wall | <1 mm | 0 |
| thickness | 1-3 mm | 1 |
| | More than 3 mm | 2 |

TABLE 9
SCORING OF HISTOLOGICAL COLITIS

| | | |
|---|---|---|
| Abnormalities of | None (normal) | 0 |
| mucosal | Minimal for focal, not exceeding lamina propria | 1 |
| architecture | Mild abnormality, cystic dilation/aberrant crypts | 2 |
| | Moderate or multifocal abnormalities | 3 |
| | Severe, entire crypt and epithelium lost | 4 |
| Extent of | None | 0 |
| inflammation | Minimal for focal, scattered cells (<10%) | 1 |
| | Mild (10-25%) | 2 |
| | Moderate, inflammatory cells extending | 3 |
| | into the submucosa | |
| | Severe, transmural leukocyte infiltrate | 4 |
| | from mucosa to serosa | |
| Erosion or | No erosion, ulceration, or granulation tissue | 0 |
| ulceration | Minimal or focal, not exceeding lamina propria | 1 |
| | Unequivocal erosion | 2 |
| | Moderate, ulceration | 3 |
| | Severe, ulceration or granulation tissue | 4 |
| Epithelial | Complete regeneration or normal tissue | 0 |
| regeneration | Almost complete regeneration | 1 |
| | Regeneration with crypt depletion | 2 |
| | Surface epithelium not intact | 3 |
| | No tissue repair | 4 |
| Percent | None | 0 |
| involvement | 1-25% | 1 |
| | 26-50% | 2 |
| | 51-75% | 3 |
| | 76-100% | 4 |

### Characterisation of IL-6 and IL-6/IL-6R-Mediated STAT3 Suppressive Activity.

To assess IL-6 and IL-6/IL-6R-mediated STAT3 suppressive activity, three independent colonies were inoculated and grown until early stationary phase. Then, each seed culture broth was used to inoculate two technical replicates each generating six technical replicates from three biological replicates. The technical replicates were grown until early stationary phase and then cell free culture supernatant was harvested as previously described (Giri et al., 2019). Culture supernatants were size fractionated by passing through a 3 kDa CENTRICON® column according to the manufacturer's (Merck Millipore) instructions.

IL-6- and IL-6/IL-6R mediated STAT3 activity was assessed using the HEK Blue IL-6 cell line (Invivogen). Briefly, 50,000 cells per well were seeded in triplicate in a 96-well plate 24 hours prior to the start of the assay. Bacteria supernatant or sterile bacterial medium were added to the cells at a final concentration of 10% v/v for a 60-minute pretreatment. Then, recombinant human IL-6 (2 ng/mL) or IL-6/IL-6R complex (400 ng/mL)(R&D systems) was added and the cells were incubated at 37° C. for 24 hours. The ability of the supernatants to suppress IL-6 or IL-6/IL-6R mediated STAT3 activation was compared to the Janus kinase inhibitor tofacitinib (10 μM). After 24 hours, the STAT3 regulated SEAP reporter activity was assessed using Quanti Blue solution as recommended by the manufacturer (Invivogen). Results are the average of three (IL-6) or one (IL-6/IL-6R) independent experiments. Cytotoxicity was assessed using the CellTiter-Glo® 2.0 Cell Viability Assay (Promega, Australia) as recommended by the manufacturer.

### Characterisation of IL-23-Mediated STAT3 Suppressive Activity.

I. colisanans MH27-3 supernatant samples were prepared using a standard protocol. Briefly, three independent colonies of the bacterium of interest were inoculated and grown until early stationary phase. Then, each seed culture broth from three biological replicates was used to inoculate two technical replicates thus generating six technical replicates. The technical replicates were grown until early stationary phase and then raw cell-free culture supernatant was harvested. Culture supernatants were also size fractionated by passing through a 3 kDa CENTRICON® column according to the manufacturer's (Merck Millipore) instructions. R. bromii MCB950 supernatant samples were similarly prepared except that two biological replicates were used to generate four technical replicates.

IL-23-mediated STAT3 activity was assessed using the HEK Blue IL-23 cell line (Invivogen). Briefly, the HEK-Blue IL-23 reporter cell line is stably transfected with IL23R, STAT3 and a Secreted Embryonic Alkaline Phosphatase (SEAP) reporter gene. Stimulation with IL-6 results in STAT3 dependent expression of SEAP. SEAP is secreted into the medium and the extent of SEAP expression can be quantified using QUANTI-Blue solution. HEK-Blue IL-23 cells were cultured in DMEM supplemented with 10% FBS accordingly to the manufacturer's directions.

For the IL23-STAT3 assay, 50,000 cells of HEK-BIue™ IL-23 cell line were grown overnight in 96 wells plates. Supernatants were initially tested at the final concentration of 10%, 25% or 50% v/v with IL-23 at a final concentration of 5 ng/mL. For all the experiments and treatments demonstrated herein, supernatants were tested at the final concentration of 25% (v/v). IL-23 was tested at a final concentration of 5 ng/mL while tofacitinib was tested at a final concentration of 5 μM. IL-23 alone and IL-23 with tofacitinib were included in the assay as positive and negative controls for IL-23 signalling. Treated cells were incubated at 37° C. for 6 hours.

SEAP production was quantified using QUANTI-BIue™ solution and recorded as optical density (OD) at 630 nm using a PHERAstar FS plate reader (BMG Labtech). Inhibition of the STAT3 signalling by bacterial supernatants was compared to the STAT3 signalling activation of its relative medium control. Of note, in some cases, the medium control itself showed some level of STAT3 signalling inhibition, however the effect of the relative leads was in many cases more potent than the medium.

All experiments were performed at least in triplicate. All data were generated from at least three technical replicates and at least three biological replicates. T-test and ANOVA test were performed accordingly using GraphPad Prism 9 software.

### Preparation of Bacterial Supernatant and Medium Extract.

Bacterial culture supernatants were prepared by inoculating three independent colonies of I. colisanans MH27-2 and growing the culture until early stationary phase. The three “seed cultures” were used to each inoculate 160 mL of YG/V broth in duplicate and the cultures were again incubated until early stationary phase. The culture supernatants were collected and prepared by centrifuging the culture at 4000 g for 30 minutes and then passing the cell free supernatant through Amicon Ultra-15 3 kDa centrifugal filters.

Bacterial culture supernatant extracts were prepared using an Amberlite XAD-7 resin as previously described by Colosimo et al. (supra). Briefly, activated Amberlite XAD-7 resin was added to 150 mL of 3 kDa-filtered cell-free supernatant (10% w/v), and gently shaken overnight at 4° C. The resin was collected, washed with 150 mL of deionized water, and mixed with 50 mL 100% methanol. Following 2 h incubation with gentle shaking, the methanol elution was collected. A second elution in 25 mL of 100% methanol was performed as previously described and the two elution were combined and dried with a rotary evaporator under vacuum. Dried extracts were resuspended in 100% DMSO at a 100× concentration and stored at −80° C.

Bacterial culture supernatant solid phase extraction fractions were prepared using Strata-X 33 μM Polymeric Reversed Phase columns. Briefly, after conditioning and equilibration, columns were loaded with 3 kDa-filtered cell-free supernatant, washed 4 times with an equal volume of de-ionised water and then eluted with 15, 30, 60 and 100% of acetonitrile. Each elution was dried with a rotary evaporator under vacuum and resuspended in 100% DMSO at a 100× concentration and stored at −80° C.

### LC-MS/MS-Based Metabolomic Analysis.

Targeted metabolomic analysis was carried out by MS-Omics (Denmark). Briefly, to assess the metabolites produced by I. colisanans MH27, six independent colonies were inoculated and grown until early stationary phase. Then, each seed culture broth was used to inoculate two technical replicates of YG/V generating 12 technical replicates from six biological replicates. The technical replicates were grown until early stationary phase and then cell free culture supernatant was harvested following centrifugation at 12,550 g for 3 minutes in an anaerobic chamber. Culture supernatants were snap frozen on dry ice and then stored at −80° C. until testing.

The analysis was carried out using a Thermo Scientific Vanquish LC coupled to Thermo Q Exactive HF MS. An electrospray ionization interface was used as ionization source. Analysis was performed in negative and positive ionization mode. The UPLC was performed using a slightly modified version of the protocol described by Catalin et al. (UPLC/MS Monitoring of Water-Soluble Vitamin Bs in Cell Culture Media in Minutes, Water Application note 2011, 720004042en). Peak areas were extracted using Compound Discoverer 3.1 (Thermo Scientific). Identification of compounds were performed at four levels; Level 1: identification by retention times (compared against in-house authentic standards), accurate mass (with an accepted deviation of 3 ppm), and MS/MS spectra, Level 2a: identification by retention times (compared against in-house authentic standards), accurate mass (with an accepted deviation of 3 ppm). Level 2b: identification by accurate mass (with an accepted deviation of 3 ppm), and MS/MS spectra, Level 3: identification by accurate mass alone (with an accepted deviation of 3 ppm).

The untargeted metabolomic analysis was carried out using a Thermo scientific UltiMate 3000 UHPLC coupled to Thermo Q Exactive Plus MS. A gradient from 3 to 95% acetonitrile with 0.1% formic acid was performed at a flow rate of 0.25 μL/min for a total of 85 minutes. An electrospray ionization interface was used as ionization source. MS data were acquired from an MS1 survey over m/z 100-1,500 at 35,000 resolution in data dependent acquisition mode, with up to ten MS2 scans in CID/HCD mode acquired per cycle with 35,000 resolution and one microscan in positive or negative mode. The maximum injection time was set to 100 ms, and the MS2 precursor window was set to 1.2 m/z. The normalized collision energy was set to stepped energy mode where normalized collision energies (NCEs) from 20, 30 and 40% were combined, with the default charge state set as z=1. MS2 experiments were set to be automatically triggered within 5 to 10 s of their first occurrence at the apex of a peak. The dynamic exclusion time was set to 10 s. Ion species with unassigned charge states and isotope peaks were excluded. Further metabolic profile analysis and key metabolite identification were performed using the GNPS platform as described by Aron et al (9).

### Assessment of Barrier Function.

The ability of I. colisanans MH27 to prevent loss of barrier integrity was assessed by measuring the transepithelial electrical resistance (TEER) across confluent monolayers of T84 gut epithelial cells. T84 cells were purchased from CellBank Australia and cultured in Dulbecco's Modified Eagle Medium Nutrient Mixture 12 (DMEM/F12; ThermoFisher Scientific, Waltham, MA, USA) supplemented with 5% foetal bovine serum and 1% Penicillin-Streptomycin. T84 cells were seeded into 24-well Millicell polycarbonate cell culture inserts with 0.4 μm pore size (PSHT010R5) at a density of 60.000 cells per well in 400 μL medium, and 24 ml of medium was added into the single-well feeder tray. Medium changes of both compartments were performed every second day. After 7 days of culture, the upper part of the plate assembly with cell culture inserts were transferred from the feeder tray to a 24-well receiver tray (PSMW010R5), with each well containing 800 μL media. TEER values of each well were measured daily using the Millicell ERS-2 Voltohmmeter. Experiments were started once the cells in all wells reached stable TEER readings above 1500 n.

In the first experiment, 3 kDa-filtered bacterial medium (YG/V) or 3 kDa-filtered I. colisanans MH27 bacterial culture supernatant diluted to 10% v/v in T84 medium was added to the apical compartment and pre-treated for one hour. Then, IFNγ (50 ng/ml) was added to the basal compartment to disrupt barrier integrity. After 48 hours of IFNγ treatment, the medium containing IFNγ in the basolateral compartment was removed, the wells washed twice with warm PBS, and fresh T84 medium was added. From then on, the medium in the lower compartment was replaced every 24 hours. Likewise, the YG/V and I. colisanans MH27-2 based treatments in the apical compartment were refreshed every 24 hours, and the TEER values were measured immediately prior to and directly after the treatments. Data from two biological replicates with duplicate measurements each were presented as the percentage difference in TEER value compared to the control (untreated T84 cells). Statistical significance was determined by unpaired t test.

In a second experiment, bacterial medium extract (YG/V) or I. colisanans MH27-2 bacterial extract at 2×, 1× and 0.5× concentration was added to the apical compartment. IL-6 (10 ng/mL) was then added to disrupt barrier integrity and removed after 96 hours treatment. Every 24 hours, the treatments and medium were refreshed, and the TEER values were measured in duplicate. Data from two biological replicates with duplicate measurements each were presented as the percentage difference in TEER value compared to the control (untreated T84 cells). Statistical significance was determined by unpaired t test.

### Assessment of Tight Junction-Associated Protein Expression.

The ability of I. colisanans MH27-1, MH27-2, and MH27-3 to prevent IFNγ-mediated loss of Zonula Occludens 1 (ZO1) expression was assessed through immunofluorescence staining and confocal microscopy. T84 cells were cultured as described above and seeded onto microscopy slides in 24-well plates at a density of 150.000 cells per well in 800 μL of medium. Cells were grown for three weeks, and media changed every second day. For I. colisanans, cells were pre-treated with 3 kDa-filtered extract control (YG/V), or 3 kDa-filtered complete- or fractionated I. colisanans MH27 bacterial extracts diluted to 1× in medium and incubated at 37° C. with 5% CO₂ overnight after which 100 ng/mL IFNγ was added to select wells. For metabolites produced by I. colisanans, T84 cells were pre-treated with 1 mM ornithine, indole-3-acrylic acid or indole-3-proprionic acid for 18 hours prior to stimulation with IFNγ for 48 hours. Cells were incubated for a further 48 h then fixed with 4% paraformaldehyde for 15 minutes and washed three times with PBS and stained protected from light. To this end, coverslips were first incubated for 10 minutes with 0.1% Triton X-100 in 5% BSA/PBS. Cells were washed three times with PBS and incubated for 2 hours in 5% BSA/PBS. Next, they were washed three times with PBS and incubated with primary rabbit anti-human ZO1 antibody (5 μg/mL, Invitrogen) in 5% BSA/PBS for 2 hours, and then again washed three times with PBS. Cells were incubated with Alexa Fluor® 488-conjugated goat anti-rabbit IgG H&L secondary antibody (1:2000, ThermoFisher Scientific) and DAPI (1:1000, ThermoFisher Scientific) in 5% BSA/PBS for 1 h. Coverslips were washed three times with PBS, once with ultrapure water, and then mounted using ProLong Gold Antifade Mountant (Invitrogen). Image acquisition was performed using an inverted and fully motorised Nikon/Spectral Spinning Disc Confocal microscope (X-1 Yokogawa spinning disc with Borealis modification) with a 60×NA 1.49 Plan Apochromat oil immersion objective lens (Nikon). Images were acquired across the cell using a coupled device (CCD) camera (Andow Clara) and the Nikon elements imaging software (Nikon, Version 4.40). For each condition, four separate images were captured. Maximum projection images were assembled, and relative brightness of stained cells quantified and calculated for each image using Fiji ImageJ (Version 1.53t) and normalised to unstimulated control cells. Experimental raw data can be found in Appendices 3-5. Statistical significance was determined by one-way ANOVA and Dunnett's correction for multiple comparison.

### PBMC Isolation and Stimulation

PBMCs were isolated from peripheral blood of healthy donors. The cells were adjusted to a density of 2×10⁶ cells/mL in RPMI 1640 (supplemented with gentamicin (150 g/mL), 2 mM L-glutamine and 10% heat inactivated FBS) and seeded in 24-well tissue culture plates. Following overnight incubation at 37° C. in a 5% CO₂ incubator, 50 μL of live I. colisanans strain MH27-2 was added to give a multiplicity of infection of 1:1. Supernatants were collected and clarified by centrifugation following 24 h of stimulation at 37° C. in a 5% CO₂ incubator. Supernatants were stored at −80° C. until required. IL-10 and IL-12 were quantified using an Ella Automated Immunoassay System according to the manufacturer's instructions (Protein Simple).

### Assessment of NF-κB Modulating Activity

NF-κB activity was assessed using a custom LS174T goblet cell like cell line stably transfected with an NF-κB reporter lentivirus with Firefly luciferase under the control of a minimal (m)CMV promoter and tandem repeats of the NF-kB transcriptional response element. Stimulation with TNF results in NF-κB-p65 dependent expression of luciferase which can be quantified using Pierce Firefly Luc One-Step Glow assay kit (Thermo Scientific). LS174T-NF-κB cells were cultured in DMEM supplemented with 10% FBS. For the NF-κB assay, 50,000 cells of LS174T-NF-κB cell line were grown overnight in 96 wells plates. Cells were pre-treated with bacterial supernatants at a starting concentration of 25% v/v at 37° C. for 30 minutes. Then, TNF was added to a final concentration of 50 ng/ml. TNF alone and TNF plus the NF-κB inhibitor indole-3-carbinol (I3C) at a final concentration of 5 mM were included in the assay as positive and negative control for NF-κB signalling. Treated cells were incubated 37° C. for 7 hours. Luciferase activity was quantified using a Pierce Firefly Luc One-Step Glow assay kit and recorded using a PHERAstar FS plate reader (BMG Labtech). Modulation of NF-κB signalling by bacterial supernatants was compared to the NF-κB signalling activation of its relative medium control.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### REFERENCES

Arumugam, M. et al., Enterotypes of the human gut microbiome. Nature 473 (7346): 174-180 (2011).
Azad et al., Probiotic supplementation during pregnancy or infancy for the prevention of asthma and wheeze: systematic review and meta-analysis. BMJ. (2013) 347:f6471.
Benham H, Rehaume L M, Hasnain S Z, Velasco J, Baillet A C, Ruutu M, et al. Interleukin-23 Mediates the Intestinal Response to Microbial 0-1,3-Glucan and the Development of Spondyloarthritis Pathology in SKG Mice. Arthritis & Rheumatology. 2014; 66(7):1755-67.
Brestoff J. R. and Atris D., Commensal bacteria at the interface of host metabolism and the immune system. Nat. Immunol., 14: 676-684 (2013).
Britto S L, Krishna M, Kellermayer R. Weight loss is a sufficient and economical single outcome measure of murine dextran sulfate sodium colitis. FASEB bioAdvances. 2019; 1(8): 493-7.
Chan, W., Chen, A., Tiao, D., Selinger, C. & Leong, R. Medication adherence in inflammatory bowel disease. Intest. Res. 15, 434 (2017).
Chaumeil, P. A., Mussig, A. J., Hugenholtz, P., and Parks, D. H., GTDB-Tk: A Toolkit to Classify Genomes with the Genome Taxonomy Database. (2020) Bioinformatics 36 (6): 1925-27.
Cheluvappa et al., (2014) Clin. Exp. Immunol. 175(2): 316-22.
Colosimo D A, Kohn J A, Luo P M, Piscotta F J, Han S M, Pickard A J, et al. Mapping interactions of microbial metabolites with human G-protein-coupled receptors. Cell Host & Microbe. 2019; 26(2): 273-82.e7.
D'Haens, G, Sandborn, WJ, Feagan, B G, et al. A review of activity indices and efficacy end points for clinical trials of medical therapy in adults with ulcerative colitis. Gastroenterology. 2007; 132: 763-86.
Fabro et al., (2015) Immunobiology, 220(1): 124-35.
Friedrich M, Pohin M, Powrie F. Cytokine Networks in the Pathophysiology of Inflammatory Bowel Disease. Immunity. 2019; 50(4):992-1006.
Foligne B, Nutten S, Grangette C, Dennin V, Goudercourt D, Poiret S, et al. Correlation between in vitro and in vivo immunomodulatory properties of lactic acid bacteria. World Jour Gastroenterol. 2007; 13(2): 236-43.
GBD 2017 Inflammatory Bowel Disease Collaborators, The global, regional, and national burden of inflammatory bowel disease in 195 countries and territories, 1990-2017: a systematic analysis for the Global Burden of Disease Study 2017; Lancet, 5(1): 17-30.
Geva-Zatorsky, N. et al. Mining the human gut microbiota for immunomodulatory organisms. Cell 168, 928-943 (2017).
Giri R, Hoedt E C, Shamsunnahar K, McGuckin M A, Morrison M, Capon R J, et al. Secreted microbial metabolites modulate gut immunity and inflammatory tone. bioRxiv. 2019:2019.12.16.861872.
Goldin B. R., Gorbach S. L. Clinical indications for probiotics: an overview. Clin Infect Dis. (2008) 46 Suppl 2: S96-100.
Handbook of Pharmaceutical Excipients, 2nd Edition, (1994), Edited by A Wade and PJ Weller.
Hu X, li J, Fu M, Zhao X, Wang W. The JAK/STAT signaling pathway: from bench to clinic. Signal Transduction and Targeted Therapy. 2021; 6(1):402.
Kabat, A. M., Srinivasan, N. & Maloy, K. J. Modulation of immune development and function by intestinal microbiota. Trends Immunol. 35, 507-517 (2014).
Kailasapathy et al. (2002) Curr. Issues Intest. Microbiol. 3(2):39-48.
Karagozian, R. & Burakoff, R. The role of mesalamine in the treatment of ulcerative colitis. Ther. Clin. Risk Manag. 3, 893 (2007).
Kim et al., Intestinicoccus butyricigenes sp. nov., a butyrate-producing bacterium isolated from human faeces, J. Microbio. 57(1): 38-44; (2019).
Liu G, Mateer S W, Hsu A, Goggins B J, Tay H, Mathe A, et al. Platelet activating factor receptor regulates colitis-induced pulmonary inflammation through the NLRP3 inflammasome. Mucosal Immunol. 2019; 12(4): 862-73.
Mallone R., Mannering S. I., Brooks-Worrell B. M., Durinovic-Belló I., Cilio C. M., Wong F. S., et al. Isolation and preservation of peripheral blood mononuclear cells for analysis of islet antigen-reactive T cell responses: position statement of the T-Cell Workshop Committee of the Immunology of Diabetes Society. Clin Exp Immunol. 2011; 163(1): 33-49.
Marks E, Goggins B J, Cardona J, Cole S, Minahan K, Mateer S, et al. Oral delivery of prolyl hydroxylase inhibitor: AKB-4924 promotes localized mucosal healing in a mouse model of colitis. Inflamm Bowel Dis. 2015; 21(2): 267-75.
Masco, L. et al., Identification of Bifidobacteriurn species using rep-PCR fingerprinting, Systematic and Applied Microbioloogy, 26(4): 557-563 (2003).
McLornan D P, Pope J E, Gotlib J, Harrison C N. Current and future status of JAK inhibitors. Lancet. 2021; 398 (10302):803-16.
McSweeney C. S., Denman S. E., Mackie R. I., Rumen bacteria. In: Makkar H P S; Methods in gut microbial ecology for ruminants. 23-37 (2005).
Meddings, J. B., Jarand, J., Urbanski, S. J., Hardin, J., Gall, D. G. Increased gastrointestinal permeability is an early lesion in the spontaneously diabetic BB rat. Am. J. Physiol. 276, G951-G957 (1999).
Mitropoulou et al. (2013) J. Nutr. Metab. (2013) 716861.
Remington's Pharmaceutical Sciences, Mack Publishing Co. (A. R. Gennaro edit. 1985).
Monteleone et al., (2011) BMC Medicine. 2011, 9:122.
Parks, D. H., Chuvochina, M., Waite, D. W., Rinke, C., Skarshewski, A., Chaumeil, P. A., and Hugenholtz, P. A Standardized Bacterial Taxonomy Based on Genome Phylogeny Substantially Revises the Tree of Life. (2018) Nature Biotechnology.
Salas A, Hernandez-Rocha C, Duijvestein M, Faubion W, McGovern D, Vermeire S, et al. JAK-STAT pathway targeting for the treatment of inflammatory bowel disease. Nature Reviews Gastroenterology & Hepatology. 2020; 17(6): 323-37.
Sapone, A. et al., Zonulin upregulation is associated with increased gut permeability in subjects with type 1 diabetes and their relatives. Diabetes 55, 1443-1449 (2006).
Schieck et al., (2014) J. Allergy Clin. Immunol. 133(3): 888-91.
Schindler et al., JAK-STAT signaling: from interfeons to cytokines, J. Biol. Chem. (2007) 282(28): 20059-63.
Schroeder, KW, Tremaine, WJ, Ilstrup, DM. Coated oral 5-aminosalicylic acid therapy for mildly to moderately active ulcerative colitis. A randomized study. N. Engl. J. Med. 1987; 317:1625-9.
Sokol H, Pigneur B, Watterlot L, Lakhdari O, Bermudez-Humaran L G, Gratadoux J J, et al. Faecalibacterium prausnitzii is an anti-inflammatory commensal bacterium identified by gut microbiota analysis of Crohn disease patients. Proc. Natl. Acad. Sci. USA. 2008; 105(43):16731-6.
Sråtková, D. et al., Efficiency of PCR-based methods in discriminating Bifidobacterium longum ssp. Longurn and Bifidobacterium longum ssp. infantis strains of human origin. J. Microbiol, Methods, (2011) 87(1): 10-6.
United States Patent Publication No. 2016/0067188.
Yamamoto A, Itoh T, Nasu R, Nishida R. Effect of sodium alginate on dextran sulfate sodium- and 2,4,6-trinitrobenzene sulfonic acid-induced experimental colitis in mice. Pharmacology. 2013; 92(1-2): 108-16.
Yang F, Wang D, Li Y, Sang L, Zhu J, Wang J, et al. Th1/Th2 Balance and Th17/Treg-Mediated Immunity in relation to Murine Resistance to Dextran Sulfate-Induced Colitis. Journal of immunology research. 2017; 2017:7047201.
Ye et al., (2015) PLoS One, 10(1): e0117704.
Yin et al., (2014) Immunogenetics, 66(3): 215-8.

1. A composition comprising a cell or biologically pure culture of the Intestinicoccus colisanans strain deposited under accession number V21/015887 or V21/015888, or a derivative thereof.

2. (canceled)

3. (canceled)

4. (canceled)

5. A composition comprising a bacterial strain with a 16S rRNA sequence that is at least about 99%, 99.1%, 99.2%, 99.3%, 99.4%, 99.5%, 99.6%, 99.7%, 99.8%, or 99.9%, identical to any one of SEQ ID NOs: 1, 2 or 7-10, or which has a 16S rRNA gene sequence represented by any one of SEQ ID NOs: 1, 2 or 7-10.

6. The composition of claim 1, further comprising a pharmaceutically acceptable excipient, diluent, or carrier.

7. (canceled)

8. A pharmaceutical composition comprising a bacterial strain that is a phylogenetic descendant of the MRCA of I. colisanans and I. sp002305575, together with a pharmaceutically acceptable carrier, diluent, or excipient.

9. The pharmaceutical composition of claim 8, wherein the MRCA is defined at node 23596 of the bac120 phylogenetic tree from the Genome Taxonomy Database (GTDB) release 89.

10. The composition of claim 1, wherein the bacterial strain is at least partially isolated.

11. The composition of claim 1, wherein the bacterial strain is viable or non-viable.

12. The composition of claim 1, wherein the composition is in a dried form.

13. The composition of claim 1, wherein the composition is formulated in a capsule, a tablet, a pill, a troche, a lozenge, a powder, or a granule.

14. The composition of claim 1, wherein the composition is dried by lyophilisation, spray drying, fluidized bed drying, vacuum drying, or a combination thereof.

15. (canceled)

16. The composition of claim 1, further comprising a prebiotic.

17. The composition of claim 1, further comprising one or more additional bacterial strains.

18. (canceled)

19. (canceled)

20. The composition according to claim 1, wherein the bacterial strain produces an agent that attenuates or impairs signal transducer and activator of transcription 3 (STAT3) signalling in a cell.

21. The composition according to claim 20, wherein the agent is a small molecule, peptide, or nucleotide.

22. The composition according to claim 20, wherein the agent is released by the bacteria.

23. The composition according to claim 20, wherein the agent binds specifically to any one of STAT3, JAK2, TYK, or IL-23.

24. The composition according to claim 1, wherein the bacterial strain produces one or more metabolites selected from cyclo(-Phe-Pro), indole-3-lactic acid, allopurinol, propionylcarnitine, pyrogallol, 3-(2-hydroxyethyl)indole, N-acetyl-cysteine, tryptophol, indole-3 propionic acid, ornithine, acetate and/or a combination thereof.

25. The composition according to claim 1, wherein the bacterial strain is of the species I. colisanans.

26.-131. (canceled)
